(19) United States
(12) Patent Application Publication
(10) Pub. No.: US 2010/0056531 A1
(43) Pub. Date: Mar. 4, 2010

(54) ALKYL-SUBSTITUTED 3' COMPOUNDS HAVING 5-HT6 RECEPTOR AFFINITY

(75) Inventors: Mihaela Diana Danca, Mendham, NJ (US); Robert Dunn, Towaco, NJ (US); Ashok Tehim, Ridgewood, NJ (US); Wenge Xie, Mahwah, NJ (US)

Correspondence Address: DARBY & DARBY P.C., P.O. BOX 770, Church Street Station, New York, NY, 10008-0770, US

(73) Assignee: Memory Pharmaceuticals Corporation, Montvale, NJ (US)

(21) Appl. No.: 12/502,121

(22) Filed: Jul. 13, 2009

Related U.S. Application Data
(60) Provisional application No. 61/091,133, filed on Aug. 22, 2008.

(51) Int. Cl. A61K 31/498; C07D 401/14; A61P 25/00; A61P 25/28; A61P 25/30; A61P 1/00; A61P 25/16; A61P 25/08; A61P 25/24; A61P 25/06; A61P 25/22; A61P 25/18; A61P 31/18; A61P 9/00

(52) U.S. Cl. 514/249; 544/349

(57) ABSTRACT

The present disclosure provides compounds having affinity for the 5-HT₆ receptor which are of the formula (I):

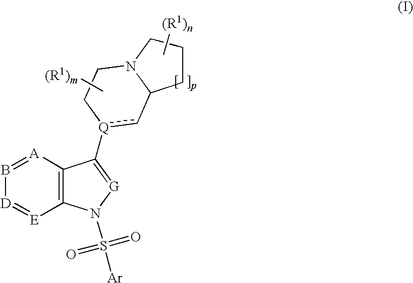

wherein R¹, A, B, D, E, G, Q, Ar, n, m, and p are as defined herein. The disclosure also relates to methods of preparing such compounds, compositions containing such compounds, and methods of use thereof.

This application claims priority to U.S. Provisional Application 61/091,133 which was filed Aug. 22, 2008, and is hereby incorporated by reference in its entirety.

### BACKGROUND OF THE INVENTION

The human 5-hydroxytryptamine-6 (5-HT₆) receptor, one of the most recently cloned serotonergic receptors, is a 440-amino acid polypeptide with seven transmembrane spanning domains typical of the G-protein-coupled receptors. It is one of the 14 receptors that mediate the effects of the neurotransmitter 5-hydroxytryptamine (5-HT, serotonin) (Hoyer, et al., Neuropharmacology, 1997, 36:419). Within the transmembrane region, the human 5-HT₆ receptor shows about 30-40% homology to other human 5-HT receptors and is found to be positively coupled to adenylyl cyclase.

The prominent localization of 5-HT₆ receptor mRNA in the nucleus accumbens, striatum, olfactory tubercle, substantia nigra, and hippocampus of the brain (Ward, et al., Neuroscience, 1995, 64:1105) together with its high affinity for several therapeutically important antipsychotics and antidepressants, suggest a possible role for this receptor in the treatment of schizophrenia and depression. In fact, the prototypic atypical antipsychotic agent clozapine exhibits greater affinity for the 5-HT₆ receptor than for any other receptor subtype (Monsma, et al., J. Pharmacol. Exp. Ther., 1994, 268:1403).

Although the 5-HT₆ receptor has a distinct pharmacological profile, in vivo investigation of receptor function has been hindered by the lack of selective agonists and antagonists. Recent experiments demonstrated that chronic intracerebroventricular treatment with an antisense oligonucleotide, directed at 5-HT₆ receptor mRNA, elicited a behavioral syndrome in rats consisting of yawning, stretching, and chewing. This syndrome in the antisense-treated rats was dose-dependently antagonized by atropine (a muscarinic antagonist), implicating 5-HT₆ receptor in the control of cholinergic neurotransmission. Therefore, 5-HT₆ receptor antagonists may be useful for the treatment of memory dysfunction (Bourson, et al., J. Pharmacol. Exp. Ther., 1995, 274:173), and to treat other central nervous system (CNS) disorders.

The high affinity of a number of antipsychotic agents for the 5-HT₆ receptor, in addition to its mRNA localization in striatum, olfactory tubercle and nucleus accumbens suggests that some of the clinical actions of these compounds may be mediated through this receptor. Compounds which interact with, stimulate, or inhibit the 5-HT₆ receptor are commonly referred to as 5-HT₆ ligands. In particular, 5-HT₆ selective ligands have been identified as potentially useful in the treatment of certain CNS disorders such as Parkinson's disease, Huntington's disease, anxiety, depression, manic depression, psychoses, epilepsy, obsessive compulsive disorders, migraine, Alzheimer's disease (enhancement of cognitive memory), sleep disorders, feeding disorders such as anorexia and bulimia, panic attacks, attention deficit hyperactivity disorder (ADHD), attention deficit disorder (ADD), withdrawal from drug abuse such as cocaine, ethanol, nicotine and benzodiazepines, schizophrenia, bipolar disorder, and also disorders associated with spinal trauma and/or head injury such as hydrocephalus. Such compounds are also expected to be of use in the treatment of certain gastrointestinal (GI) disorders such as functional bowel disorder and irritable bowel syndrome.

Therefore, it is advantageous to provide compounds which are useful as therapeutic agents in the treatment of a variety of central nervous system disorders modulated by the 5-HT₆ receptor.

It is also advantageous to provide therapeutic methods and pharmaceutical compositions useful for the treatment of central nervous system disorders modulated by the 5-HT₆ receptor.

The following patents and publications also provide relevant background to the present invention. All references cited below are incorporated herein by reference in their entirety and to the same extent as if each reference was individually incorporated by reference. U.S. Pat. Nos. 6,100,291, 6,133,287, 6,191,141, 6,251,893, 6,686,374, 6,767,912, 6,897,215, 6,903,112, 6,916,818, and 7,268,127, and Published U.S. Application No. 2008/0039462.

### SUMMARY OF THE INVENTION

The present invention relates to novel compounds that have affinity, preferably selectively, for the serotonin 5-HT₆ receptor, methods of use thereof, and the synthesis thereof.

Still further, the present invention provides methods for synthesizing compounds with such activity and selectivity, as well as methods of and corresponding pharmaceutical compositions for treating a disorder (e.g. a mood disorder and/or a cognitive disorder) in a patient, wherein the disorder is modulated by the 5-HT₆ receptor.

Pharmaceutical compositions containing the novel compounds of the present invention can be sued for the treatment of diseases or condition involving modulation of the 5-HT6 receptor. Such diseases and conditions include, but are not limited central nervous system disorders (CNS), memory/cognitive impairments, withdrawal from drug abuse, psychoses, gastrointestinal (GI) disorders, and polyglutamine-repeat diseases.

### DETAILED DESCRIPTION OF THE INVENTION

The present invention includes compounds of formula (I):

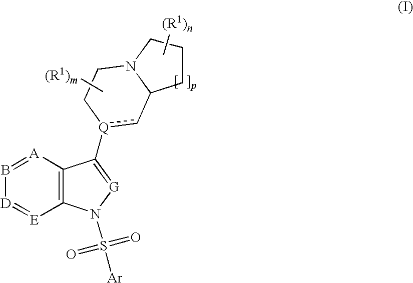

wherein
A, B, D, E and G are each N, CH or CR², wherein at least one of A, B, D, E and G is N;
Q is independently N, C or CH;
[symbol] represents a single bond or a double bond;
m is 0, 1, or 2;
n is 0, 1, or 2;
p is 1, 2 or 3;
R¹ is, in each instance independently, H, OH, oxo, phenyl, or heteroaryl, alkyl having 1 to 8, preferably 1 to 4 carbon atoms (e.g., CH₃), or alkoxy having 1 to 8, preferably 1 to 4 carbon atoms (e.g., OCH₃), each of which is unsubstituted or substituted one or more times with halogen, OH, C₁₋₄-alkyl, C₁₋₄-alkoxy, oxo, phenyl, heteroaryl, or any combination thereof;
R² is, in each instance independently, halogen (e.g., F), nitro, cyano, or NR³R³,
   alkyl having 1 to 8, preferably 1 to 4 carbon atoms, which is unsubstituted or substituted one or more times with halogen, C₁₋₄-alkyl, C₁₋₄-alkoxy, oxo, or any combination thereof (e.g., CHF₂, or CF₃),
   alkoxy having 1 to 8, preferably 1 to 4 carbon atoms, which is unsubstituted or substituted one or more times with halogen, (e.g., —OCF₃ or —OCHF₂), C₁₋₆ alkyl, phenyl, or heteroaryl,
   an aryl group having 6 to 10 ring atoms, which is unsubstituted or substituted one or more times by halogen, hydroxy, C₁₋₄-alkyl, C₁₋₄-alkoxy, cyano, halogenated C₁₋₄-alkyl (e.g., trifluoromethyl), nitro, or any combination thereof (e.g., substituted or unsubstituted benzene, substituted or unsubstituted naphthalene),
   a heterocyclic group, which is saturated, partially saturated or unsaturated, having 5 to 10 ring atoms in which at least 1 ring atom is an N, O or S atom, which is unsubstituted or substituted one or more times by halogen, hydroxy, C₆₋₁₀-aryl, C₁₋₄-alkyl, C₁₋₄-alkoxy, cyano, halogenated C₁₋₄-alkyl (e.g., trifluoromethyl), nitro, or any combination thereof (e.g., substituted or unsubstituted morpholinyl, substituted or unsubstituted pyrrolyl, substituted or unsubstituted pyrrolidinyl, substituted or unsubstituted piperidinyl, substituted or unsubstituted pyridyl),
   —C(═O)alkyl, —C(═O)-heteroaryl, —C(═O)—NR⁷R⁷, —C(═O)-alkoxy, or —C(═O)-phenyl;
R³ is, in each instance, independently hydrogen,
alkyl having 1 to 8, preferably 1 to 4 carbon atoms, cycloalkyl having 3 to 12, preferably 3 to 8 carbon atoms, or cycloalkylalkyl having 4 to 12, preferably 4 to 8 carbon atoms, each of which is branched or unbranched and which is unsubstituted or substituted one or more times with halogen, C₁₋₄-alkyl, C₁₋₄-alkoxy, oxo, or any combination thereof (e.g., CHF₂, or CF),
an aryl group, having 6 to 10 ring atoms, which is unsubstituted or substituted one or more times by halogen, hydroxy, C₅₋₇-aryl, C₁₋₄-alkyl, C₁₋₄-alkoxy, cyano, halogenated C₁₋₄-alkyl (e.g., trifluoromethyl), nitro, or any combination thereof (e.g., substituted or unsubstituted phenyl, substituted or unsubstituted naphthyl),
a heterocyclic group, which is saturated, partially saturated or unsaturated, having 5 to 10 ring atoms in which at least 1 ring atom is an N, O or S atom, which is unsubstituted or substituted one or more times by halogen, hydroxy, C₅₋₇-aryl, C₁₋₄-alkyl, C₁₋₄-alkoxy, cyano, halogenated C₁₋₄-alkyl (e.g., trifluoromethyl), nitro, or any combination thereof (e.g., substituted or unsubstituted morpholinyl, substituted or unsubstituted pyrrolyl, substituted or unsubstituted pyrrolidinyl, substituted or unsubstituted piperidinyl, substituted or unsubstituted pyridyl),
aralkyl, heteroaralkyl, heterocyclylalkyl, —C(O)R⁶, —C(O)OR⁷, —C(O)NR⁷R⁷, —S(O)R⁶, or —S(O)₂R⁶;
Ar is selected from Formulas (a), (b), and (c):

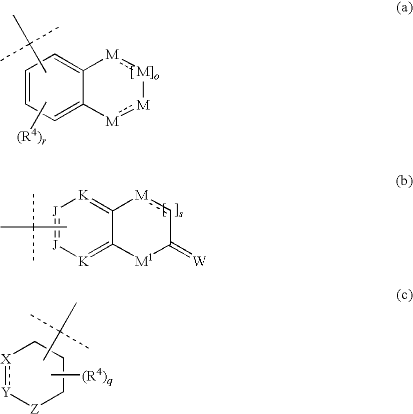

J is, in each instance independently C (attachment to sulfonyl), CH, or CR⁴;
M is, in each instance independently CH, CH₂, CR⁴, CHR⁴, N, NR⁵, O or S, wherein at least one M is not CH, CH₂, CR⁴, or CHR⁴;
M¹ is CH₂, CHR⁴, NR⁵, O or S;
K is, in each instance independently C (attachment to sulfonyl), CH, CR⁴ or N;
X, Y, and Z are each independently C (attachment to sulfonyl), CH, CR⁴, or N;
W is O, S or is absent;
o is 0 or 1;
q is 0, 1 or 2;
r is 0, 1, 2 or 3;
s is 0 or 1;
[symbol] represents a single bond or a double bond;
R⁴ is, in each instance, independently, halogen (e.g., F, Cl, or Br), C₁₋₄-alkyl, C₁₋₄-alkoxy (e.g., methoxy), —C(═O)alkyl, —C(═O)-pyridyl, cyano, amino, N—C₁₋₄-alkyl-N—C₁₋₄-acylamino, mono- or di-C₁₋₄-alkylamino, morpholinyl, pyrrolidinyl, or pyrrolidone-1-yl; wherein
   the morpholinyl, pyrrolidinyl, or pyrrolidone-1-yl group may be substituted with hydroxy, C₁₋₈-alkyl, C₁₋₄-alkoxy, or a 3 to 7-membered heterocycloalkoxy containing at least one O, S or N atom, and wherein
   each alkyl and alkoxy is independently unsubstituted or substituted one or more times by halogen (e.g., CH₃, CH₂CH₃, CHF₂, CF₃, OCF₃, or OCHF₂) or acyl;
R⁵ is, in each instance, independently hydrogen,
alkyl having 1 to 8, preferably 1 to 4 carbon atoms, cycloalkyl having 3 to 12, preferably 3 to 8 carbon atoms, alkenyl or alkynyl having 3 to 8 atoms and at least one double or triple bond located at least one carbon atom from the point of attachment, or cycloalkylalkyl having 4 to 12, preferably 4 to 8 carbon atoms, each of which is branched or unbranched and which is unsubstituted or substituted one or more times with halogen, C₁₋₄-alkyl, C₁₋₄-alkoxy, oxo, or any combination thereof (e.g., CHF₂, or CF₃),
an aryl group having 6 to 10 ring atoms, which is unsubstituted or substituted one or more times by halogen, hydroxy, C₅₋₇-aryl, C₁₋₄-alkyl, C₁₋₄-alkoxy, cyano, halogenated C₁₋₄-alkyl (e.g., trifluoromethyl), nitro, or any combination thereof (e.g., substituted or unsubstituted phenyl, substituted or unsubstituted naphthyl),
a heterocyclic group, which is saturated, partially saturated or unsaturated, having 5 to 10 ring atoms in which at least 1 ring atom is an N, O or S atom, which is unsubstituted or substituted one or more times by halogen, hydroxy, C₅₋₇-aryl, C₁₋₄-alkyl, C₁₋₄-alkoxy, cyano, halogenated C₁₋₄-alkyl (e.g., trifluoromethyl), nitro, or any combination thereof (e.g., substituted or unsubstituted morpholinyl, substituted or unsubstituted pyrrolyl, substituted or unsubstituted pyrrolidinyl, substituted or unsubstituted piperidinyl, substituted or unsubstituted pyridyl),
C₇₋₁₄-aralkyl, C₅₋₁₃-heteroaralkyl, C₅₋₁₃-heterocyclylalkyl, —C(O)R⁶, —C(O)OR⁷, —C(O)NR⁷R⁷, —S(O)R⁶, or —S(O)₂R⁶;
R⁶ is, in each instance, independently C₁₋₄-alkyl, C₃₋₈-cycloalkyl, C₄₋₁₀-cycloalkylalkyl, C₆₋₁₀-aryl, C₇₋₁₄-aralkyl, C₄₋₇-heteroaryl, C₅₋₁₃-heteroaralkyl, C₄₋₉-heterocyclyl, or C₅₋₁₃-heterocyclylalkyl;
R⁷ is, in each instance, independently hydrogen, C₁₋₄-alkyl, C₃₋₈-cycloalkyl, C₄₋₁₀-cycloalkylalkyl, C₆₋₁₀-aryl, C₇₋₁₄-aralkyl, C₄₋₇-heteroaryl, C₅₋₁₃-heteroaralkyl, C₄₋₉-heterocyclyl, or C₅₋₁₃-heterocyclylalkyl;
   and pharmaceutically acceptable salts or solvates (e.g., hydrates) thereof, or solvates of pharmaceutically acceptable salts thereof,
   provided that when Ar is (c) and X, Y and Z are each CH or CR⁴, then q is 1 or 2 and at least one R⁴ is C(═O)alkyl, —C(═O)pyridyl, cyano, amino, N—C₁₋₄-alkyl-N-acylamino, mono- or dialkylamino, morpholinyl, pyrrolidinyl, or pyrrolidone-1-yl, wherein the alkyl may be substituted or unsubstituted.

In a first embodiment, the compound contains a chiral center (i.e., at a pyrrolidinyl, or pyrrolidine moiety) and the compound is a racemic mixture of isomers about this chiral center.

In a second embodiment, the compound contains a chiral center and the compound is the [R] isomer at this chiral center.

In a third embodiment, the compound contains a chiral center and the compound is the [S] isomer at this chiral center.

In the above embodiments, where the compound has more than one chiral center, the compound may be racemic at one chiral center while having the [R] or the [S] configuration at the other chiral center(s). Alternatively, the compound may have two (or more) [R] chiral centers, two (or more) [S] chiral center(s), or a mixture of [R] and [S] chiral centers.

In a fourth embodiment, there are two R⁴ moieties on the aryl defined by (a), (b), or (c).

In one embodiment, the two R⁴s are different. In another embodiment, the two R⁴s are the same.

In a fifth embodiment, Ar is (b) and both variables J are CH.

In a sixth embodiment, G is N.

In a seventh embodiment, A is N and B, D, E, and G are CH or CR². In another embodiment, A is N and B, D, E, and G are CH. In another embodiment, one of A, B, D, and E is N and G is N. In another embodiment, A and G are N and B, D, and E are CH or CR².

In an eighth embodiment, q is 1, 2, or 3. In another embodiment, r is 1, 2, or 3.

In a ninth embodiment, p is 1 or 2.

In a tenth embodiment, each R¹ is H or C₁₋₄-alkyl. In another embodiment, each R¹ is H.

In an eleventh embodiment, each R² is halogen, nitro, cyano, C₁₋₄-alkyl, or C₁₋₄-alkoxy.

In another embodiment, each R² is a heterocyclic group in which at least one ring atome is an N or O.

In a twelfth embodiment, at least one R⁴ is a heterocyclic group. In one embodiment, at least one R⁴ is preferably a substituted or unsubstituted pyrrolidine. In another embodiment, Ar is (c), q is 1 or 2, and at least one R⁴ on the ring of (c) is a heterocyclic group.

In a thirteenth embodiment, Ar is (a), at least one M is O, and o is 1. In another embodiment, Ar is (a), one M is O and the other M variables are CH.

In a fourteenth embodiment, Ar is (b), one M is NH and the other M is O, W is ═O, and p is 1.

In a fifteenth embodiment, Ar is (c) and A is N. In another embodiment, Ar is (c), A is N, and R⁴ is H, a halogen, a C₁-C₄ alkyl, a C₁-C₄ alkoxy, cyano, or a substituted or unsubstituted heterocyclic group. In another embodiment, Ar is (c), A is N, q is 1 or 2, and at least one R⁴ is a cyano group.

In a sixteenth embodiment, Ar is (a) or (b) and at least one [symbol] in the ring of (a) or (b) is a single bond.

In a seventeenth embodiment, Ar is (a) or (b), and (a) or (b) contains at least three ring heteroatoms (e.g., at least three of M and/or K are N (M or K), NR⁵ (M), O (M) and/or S (M)).

In an eighteenth embodiment, Ar is (b) and W is present.

In a nineteenth embodiment, M is, in each instance independently CH, CH₂, N, NR⁵, O or S, wherein at least one M is not CH or CH₂.

In a twentieth embodiment, Ar is (a), (b) or (c) wherein (a), (b), and (c) are represented by the formulas (a′), (b′), and (c′):

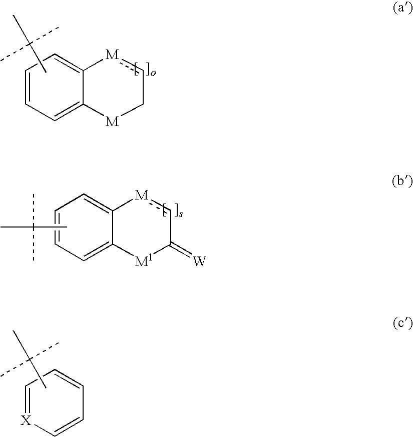

wherein M, M¹, W, X, o and s are as described above. In another embodiment, Ar is (a′), (b′), or (c′) wherein [symbol] is a single bond, W is absent, and s and o are 0.

In a twenty-first embodiment, Ar is (c), X, Y, and Z are each CH, q is 1, and R⁴ in formula (c) is a pyrrolidine or substituted pyrrolidine.

In a twenty-second embodiment, Ar contains at least one R⁴ wherein R⁴ in formula (a), (b), or (c) is C(═O)alkyl, —C(═O)pyridyl, cyano, amino, N-alkyl-N—C₁₋₄-acylamino, mono- or dialkylamino, morpholinyl, pyrrolidinyl, or pyrrolidonyl, wherein the alkyl, morpholinyl, pyrrolidinyl, orpyrrolidonyl may be substituted or unsubstituted.

In a twenty-third embodiment, the compound is a pharmaceutically acceptable salt.

In a twenty-fourth embodiment, the compound is a hydroformate salt, a phosphate salt, a dihydroiodide, a dihydrochloride monohydrate, or a hydroacetate salt.

In a twenty-fifth embodiment, the compound is a formate salt

In any of the above embodiments, the compound of formula (I) comprises a 1-H-pyrrolo[3,2-b]pyridine moiety.

In one embodiment, the compound of formula (I) is defined by the compound of formula (II):

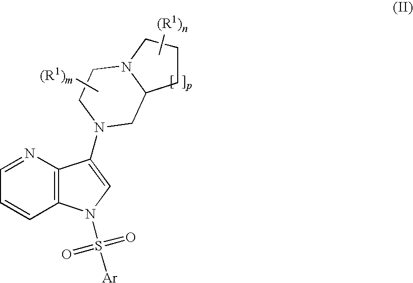

wherein R¹, Ar, n, m, and p are defined as above. In another embodiment, any one or combinations of embodiments 1-5, 8-10, and 12-22 are defined for the compound of formula (II).

In one embodiment, R⁵ is, in each instance, independently hydrogen, alkyl, cycloalkyl, cycloalkylalkyl, an aryl group, a heterocyclic group, C₆₋₁₄-aralkyl, C₅₋₁₃-heteroaralkyl, C₄₋₉. heterocyclylalkyl, —C(O)R⁶, —C(O)OR⁷, —C(O)NR^{7′}R⁷, —S(O)R⁶, or —S(O)₂R⁶.

In one embodiment, R⁵ is, in each instance, independently hydrogen, alkyl, an aryl group, a heterocyclic group, C₆₋₁₄-aralkyl, C₅₋₁₃-heteroaralkyl, C₄₋₉heterocyclylalkyl, —C(O)R⁶, C(O)OR⁷, —C(O)NR^{7′}R⁷, —S(O)R⁶, or —S(O)₂R⁶; R⁶ is, in each instance, independently linear C₁₋₄-alkyl, C₃₋₁₀-cycloalkyl, C₄₋₁₆-cycloalkylalkyl, C₅₋₇-aryl, C₆₋₁₄-aralkyl, C₅₋₇-heteroaryl, C₄₋₉-heterocyclyl, C₅₋₁₃-heteroaralkyl, or C₄₋₉-heterocyclylalkyl; R⁷ is, in each instance, independently hydrogen, linear C₁₋₄-alkyl, C₃₋₁₀-cycloalkyl, C₄₋₁₆-cycloalkylalkyl, C₅₋₇-aryl, C₆₋₁₄-aralkyl, C₅₋₇-heteroaryl, C₄₋₉-heterocyclyl, C₅₋₁₃-heteroaralkyl, or C₄₋₉-heterocyclylalkyl.

In one embodiment, the compound of formula (I) is defined wherein:
A, B, D, E and G are each N, CH or CR², wherein at least one of A, B, D, E and G is N;
Q is independently N, C or CH;
[symbol] represents a single bond or a double bond;
m is 0, 1, or 2;
n is 0, 1, or 2;
p is 1, 2 or 3;
R¹ is, in each instance independently, H, OH, oxo, phenyl, or heteroaryl, alkyl having 1 to 8, preferably 1 to 4 carbon atoms (e.g., CH₃), or alkoxy having 1 to 8, preferably 1 to 4 carbon atoms (e.g., OCH₃), each of which is unsubstituted or substituted one or more times with halogen, OH, C₁₋₄-alkyl, C₁₋₄-alkoxy, oxo, phenyl, heteroaryl, or any combination thereof;
R² is, in each instance independently, halogen (e.g., F), nitro, cyano, or NR³R³,
   alkyl having 1 to 8, preferably 1 to 4 carbon atoms, which is unsubstituted or substituted one or more times with halogen, C₁₋₄-alkyl, C₁₋₄-alkoxy, oxo, or any combination thereof (e.g., CHF₂, or CF₃),
   alkoxy having 1 to 8, preferably 1 to 4 carbon atoms, which is unsubstituted or substituted one or more times with halogen, (e.g., —OCF₃ or —OCHF₂), C₁₋₆ alkyl, phenyl, or heteroaryl,
   an aryl group having 6 to 10 ring atoms, which is unsubstituted or substituted one or more times by halogen, hydroxy, C₁₋₄-alkyl, C₁₋₄-alkoxy, cyano, halogenated C₁₋₄-alkyl (e.g., trifluoromethyl), nitro, or any combination thereof (e.g., substituted or unsubstituted benzene, substituted or unsubstituted naphthalene),
   a heterocyclic group, which is saturated, partially saturated or unsaturated, having 5 to 10 ring atoms in which at least 1 ring atom is an N, O or S atom, which is unsubstituted or substituted one or more times by halogen, hydroxy, C₆₋₁₀-aryl, C₁₋₄-alkyl, C₁₋₄-alkoxy, cyano, halogenated C₁₋₄-alkyl (e.g., trifluoromethyl), nitro, or any combination thereof (e.g., substituted or unsubstituted morpholinyl, substituted or unsubstituted pyrrolyl, substituted or unsubstituted pyrrolidinyl, substituted or unsubstituted piperidinyl, substituted or unsubstituted pyridyl),
   —C(═O)alkyl, —C(═O)heteroaryl, —C(═O)—NR⁷R⁷, —C(═O)-alkoxy, or —C(═O)-phenyl;
R³ is, in each instance, independently hydrogen,
alkyl having 1 to 8, preferably 1 to 4 carbon atoms, cycloalkyl having 3 to 12, preferably 3 to 8 carbon atoms, or cycloalkylalkyl having 4 to 12, preferably 4 to 8 carbon atoms, each of which is branched or unbranched and which is unsubstituted or substituted one or more times with halogen, C₁₋₄-alkyl, C₁₋₄-alkoxy, oxo, or any combination thereof (e.g., CHF₂, or CF₃),
an aryl group, which is an unsaturated carbocyclic ring, having 6 to 10 ring atoms, which is unsubstituted or substituted one or more times by halogen, hydroxy, C₅₋₇-aryl, C₁₋₄-alkyl, C₁₋₄-alkoxy, cyano, halogenated C₁₋₄-alkyl (e.g., trifluoromethyl), nitro, or any combination thereof (e.g., substituted or unsubstituted phenyl, substituted or unsubstituted naphthyl),
a heterocyclic group, which is saturated, partially saturated or unsaturated, having 5 to 10 ring atoms in which at least 1 ring atom is an N, O or S atom, which is unsubstituted or substituted one or more times by halogen, hydroxy, C₅₋₇-aryl, —C₁₋₄-alkyl, C₁₋₄-alkoxy, cyano, halogenated C₁₋₄-alkyl (e.g., trifluoromethyl), nitro, or any combination thereof (e.g., substituted or unsubstituted morpholinyl, substituted or unsubstituted pyrrolyl, substituted or unsubstituted pyrrolidinyl, substituted or unsubstituted piperidinyl, substituted or unsubstituted pyridyl),
aralkyl, heteroaralkyl, heterocyclylalkyl, —C(O)R⁶, —C(O)OR⁷, —C(O)NR⁷R⁷, —S(O)R⁶, or —S(O)₂R⁶;
Ar is selected from Formulas (a), (b), and (c):

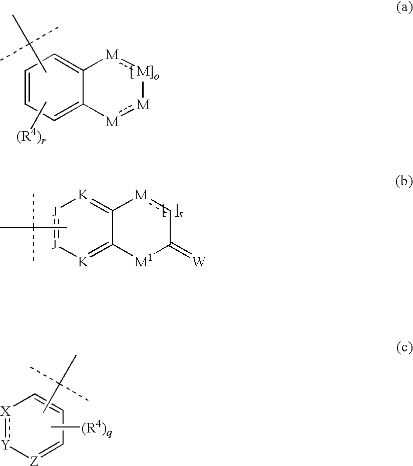

J is, in each instance independently C (attachment to sulfonyl), CH, or CR⁴;
M is, in each instance independently CH, CH₂, CR⁴, CHR⁴, N, NR⁵, O or S, wherein at least one M is not CH, CH₂, CR⁴, or CHR⁴;
M¹ is CH₂, CHR⁴, NR⁵, O or S;
K is, in each instance independently C (attachment to sulfonyl), CH, CR⁴ or N;
X, Y, and Z are each independently CH, CR⁴, or N;
W is O, S or is absent;
o is 0 or 1;
q is 0, 1 or 2;
r is 0, 1, 2 or 3;
s is 0 or 1;
[symbol] represents a single bond or a double bond;
R⁴ is, in each instance, independently, halogen (e.g., F, Cl, or Br), C₁₋₄-alkyl, C₁₋₄-alkoxy (e.g., methoxy), —C(═O)alkyl, —C(═O)-pyridyl, cyano, amino, N-alkyl-N-acylamino, mono- or dialkylamino, morpholinyl, pyrrolidinyl, or pyrrolidone-1-yl; wherein
   the morpholinyl, pyrrolidinyl, or pyrrolidone-1-yl group may be substituted with hydroxy, C₁₋₈-alkyl or C₁₋₄-alkoxy, or a 3 to 7-membered heterocycloalkoxy containing at least one O, S or N atom, and wherein
   each alkyl and alkoxy is independently unsubstituted or substituted one or more times by halogen (e.g., CH₃, CH₂CH₃, CHF₂, CF₃, OCF₃, or OCHF₂) or acyl;
R⁵ is, in each instance, independently hydrogen,
alkyl having 1 to 8, preferably 1 to 4 carbon atoms, cycloalkyl having 3 to 12, preferably 3 to 8 carbon atoms, or cycloalkylalkyl having 4 to 12, preferably 4 to 8 carbon atoms, each of which is branched or unbranched and which is unsubstituted or substituted one or more times with halogen, C₁₋₄-alkyl, C₁₋₄-alkoxy, oxo, or any combination thereof (e.g., CHF₂, or CF₃),
an aryl group, which is an unsaturated carbocyclic ring, having 6 to 10 ring atoms, which is unsubstituted or substituted one or more times by halogen, hydroxy, C₅₋₇-aryl, C₁₋₄-alkyl, C₁₋₄-alkoxy, cyano, halogenated C₁₋₄-alkyl (e.g., trifluoromethyl), nitro, or any combination thereof (e.g., substituted or unsubstituted phenyl, substituted or unsubstituted naphthyl),
a heterocyclic group, which is saturated, partially saturated or unsaturated, having 5 to 10 ring atoms in which at least 1 ring atom is an N, O or S atom, which is unsubstituted or substituted one or more times by halogen, hydroxy, C₅₋₇-aryl, C₁₋₄-alkyl, C₁₋₄-alkoxy, cyano, halogenated C₁₋₄-alkyl (e.g., trifluoromethyl), nitro, or any combination thereof (e.g., substituted or unsubstituted morpholinyl, substituted or unsubstituted pyrrolyl, substituted or unsubstituted pyrrolidinyl, substituted or unsubstituted piperidinyl, substituted or unsubstituted pyridyl),
C₆₋₁₄-aralkyl, C₅₋₁₃-heteroaralkyl, C₄₋₉heterocyclylalkyl, —C(O)R⁶, —C(O)OR⁷, —C(O)NR^{7′}R⁷, —S(O)R⁶, or —S(O)₂R⁶;
R⁶ is, in each instance, independently linear C₁₋₄-alkyl, C₃₋₁₀-cycloalkyl, C₄₋₁₆-cycloalkylalkyl, C₅₋₇-aryl, C₆₋₁₄-aralkyl, C₅₋₇-heteroaryl, C₄₋₉-heterocyclyl, C₅₋₁₃-heteroaralkyl, or C₄₋₉-heterocyclylalkyl;
R⁷ is, in each instance, independently hydrogen, linear C₁₋₄-alkyl, C₃₋₁₀-cycloalkyl, C₄₋₁₆-cycloalkylalkyl, C₅₋₇-aryl, C₆₋₁₄-aralkyl, C₅₋₇-heteroaryl, C₄₋₉-heterocyclyl, C₅₋₁₃-heteroaralkyl, or C₄₋₉-heterocyclylalkyl;
   and pharmaceutically acceptable salts or solvates (e.g., hydrates) thereof, or solvates of pharmaceutically acceptable salts thereof,

provided that when Ar is (c) and X, Y and Z are each CH or CR⁴, then at least one R⁴ is C(═O)alkyl, —C(═O)pyridyl, cyano, amino, N-alkyl-N-acylamino, mono- or dialkylamino, morpholinyl, pyrrolidinyl, or pyrrolidone-1-yl, wherein the alkyl or heteroaryl may be substituted or unsubstituted.

In one embodiment, the compound is selected from the group consisting of 1-[(2-chlorophenyl)sulfonyl]-3-(hexahydropyrrolo[1,2-a]pyrazin-2(1H)-yl)-1H-pyrrolo[3,2-b]pyridine, 3-(hexahydropyrrolo[1,2-a]pyrazin-2(1H)-yl)-1-[(3-methoxyphenyl)sulfonyl]-1H-pyrrolo[3,2-b]pyridine, and 1-[(3-chlorophenyl)sulfonyl]-3-(hexahydropyrrolo[1,2-a]pyrazin-2(1H)-yl)-1H-pyrrolo[3,2-b]pyridine, or a pharmaceutically acceptable salt, a pharmaceutically acceptable solvate, or a solvate of pharmaceutically acceptable salts thereof.

Alkyl means a straight-chain or branched-chain aliphatic hydrocarbon radical. Suitable alkyl groups include, but are not limited to the linear alkyl radicals, methyl, ethyl, propyl, isopropyl, butyl, sec-butyl, tert-butyl, pentyl, hexyl, heptyl, octyl, nonyl, decyl, undecyl, and dodecyl. Other examples of suitable alkyl groups include, but are not limited to the substituted linear alkyl radicals, 1-, 2- or 3-methylbutyl, 1,1-, 1,2- or 2,2-dimethylpropyl, 1-ethylpropyl, 1-, 2-, 3- or 4-methylpentyl, 1,1-, 1,2-, 1,3-, 2,2-, 2,3- or 3,3-dimethylbutyl, 1- or 2-ethylbutyl, ethylmethyl propyl, trimethylpropyl, methyl hexyl, dimethylpentyl, ethylpentyl, ethylmethyl butyl, dimethylbutyl, and the like. Preferably, the alkyl group will have 1 to 8 carbon atoms. In one embodiment, the non-cyclic alkyl group will have 1 to 4 carbon atoms.

Alkenyl means a straight-chain or branched-chain hydrocarbon radical where one or more —CH₂CH₂— group as defined for the alkyl chain is replaced by a —CH═CH— group. Suitable alkenyl groups include, but are not limited to, 1-propenyl, 2-propenyl, 1-butenyl, 2-butenyl, 3-butenyl, 1,3-butadienyl, and 3-methyl-2-butenyl. Preferably, the alkenyl group has 2 to 8 carbon atoms. In one embodiment, the alkenyl group has 2 to 4 carbon atoms.

Alkynyl means a straight-chain or branched-chain hydrocarbon radical where one or more —CH₂CH₂— group as defined for the alkyl chain is replaced by a —C≡C— group. Suitable alkynyl groups include, but are not limited to, 2-propynyl, 2-butynyl, 3-butynyl, and 1-methyl-3-butynyl. Preferably, the alkynyl group has 2 to 8 carbon atoms. In one embodiment, the alkynyl group has 2 to 4 carbon atoms.

Cycloalkyl refers to monocyclic, bicyclic or tricyclic saturated hydrocarbon radical having 3 to 8 carbon atoms, preferably 3 to 6 carbon atoms. Suitable cycloalkyl groups include, but are not limited to, cyclopropyl, cyclobutyl, cyclopentyl, cyclohexyl, cycloheptyl, cyclooctyl, and norbornyl. Other suitable cycloalkyl groups include, but are not limited to, spiropentyl, bicyclo[2.1.0]pentyl, bicyclo[3.1.0]hexyl, spiro[2.4]heptyl, spiro[2.5]octyl, bicyclo[5.1.0]octyl, spiro[2.6]nonyl, bicyclo[2.2.0]hexyl, spiro[3.3]heptyl, and bicyclo[4.2.0]octyl. Preferably, the cycloalkyl group has 3 to 8 carbon atoms.

Cycloalkylalkyl refers to cycloalkyl groups in which the cycloalkyl portions have preferably 3 to 8 carbon atoms, preferably 4 to 6 carbon atoms and alkyl the portions have preferably 1 to 8 carbon atoms, preferably 1 to 4 carbon atoms. Suitable examples include, but are not limited to, cyclopentylethyl and cyclopropylmethyl.

In the cases where alkyl is a substituent (e.g., alkyl substituents on aryl and heteroaryl groups) or is part of a substituent (e.g., in the alkylamino, dialkylamino, hydroxyalkyl, hydroxyalkoxy, alkylthio, alkylsulphinyl, and alkylsulphonyl substituents), the alkyl portion preferably has 1 to 12 carbon atoms, especially 1 to 8 carbon atoms, in particular 1 to 4 carbon atoms.

Acyl refers to alkanoyl radicals having 2 to 4 carbon atoms. Suitable acyl groups include, but are not limited to, formyl, acetyl, propionyl, and butanoyl.

Alkoxy means an alkyl group as defined herein attached through an oxygen linkage. The alkoxy may be branched or unbranched.

Oxo means ═O, as in C(C═O)C.

Aryl, as a group or substituent per se or as part of a group or substituent, refers to an aromatic carbocyclic radical containing 6 to 14 carbon atoms, preferably 6 to 12 carbon atoms, especially 6 to 10 carbon atoms. Suitable aryl groups include, but are not limited to, phenyl, naphthyl and biphenyl. Substituted aryl groups include the above-described aryl groups which are substituted one or more times by, for example, halogen, alkyl, hydroxy, alkoxy, nitro, methylenedioxy, ethylyenedioxy, amino, alkylamino, dialkylamino, hydroxyalkyl, hydroxyalkoxy, carboxy, cyano, acyl, alkoxycarbonyl, alkylthio, alkylsulphinyl, alkylsulphonyl, phenoxy, and acyloxy (e.g., acetoxy).

Arylalkyl, or equivalently aralkyl refers to an aryl-alkyl-radical in which the aryl and alkyl portions are in accordance with the previous descriptions. Suitable examples include, but are not limited to, benzyl, 1-phenethyl, 2-phenethyl, phenpropyl, phenbutyl, phenpentyl, and naphthalenemethyl.

Heteroaryl groups refer to unsaturated heterocyclic groups having one or two rings and a total number of 5 to 10 ring atoms wherein at least one of the ring atoms is preferably an N, O or S atom. Preferably, the heteroaryl group contains 1 to 3, especially 1 or 2, hetero-ring atoms selected from N, O and S. Suitable heteroaryl groups include, for example, furyl, benzothienyl, benzofuranyl, pyrrolyl, pyrazolyl, imidazolyl, pyridyl, pyrimidinyl, isoxazolyl, quinolinyl, azaindolyl, naphthyridinyl, thiazolyl, and the like. Preferred heteroaryl groups include, but are not limited to, furyl, benzothienyl, benzofuranyl, pyrrolyl, pyrazolyl, imidazolyl, pyridyl, pyrimidinyl, isoxazolyl, and thiazolyl.

Substituted heteroaryl groups refer to the heteroaryl groups described above which are substituted in one or more places by preferably halogen, aryl, alkyl, alkoxy, cyano, halogenated alkyl (e.g., trifluoromethyl), nitro, oxo, amino, alkylamino, and dialkylamino.

Hetereocycles are non-aromatic, saturated or partially unsaturated, cyclic groups containing at least one hetero-ring atom, preferably selected from N, S, and O, for example, 1,2,3,4,-tetrahydroquinolyl, dihydrobenzofuranyl, dihydrobenzodioxepinyl, dihydrobenzodioxinyl, dihydroindolyl, benzodioxolyl, 3-tetrahydrofuranyl, piperidinyl, imidazolinyl, imidazolidinyl, pyrrolinyl, pyrrolidinyl, morpholinyl, piperazinyl, oxazolidinyl, and indolinyl.

Heteroaralkyl (heteroarylalkyl) refers to a heteroaryl-alkyl-group wherein the heteroaryl and alkyl portions are in accordance with the previous discussions. Suitable examples include, but are not limited to, pyridylmethyl, thienylmethyl, pyrimidinylmethyl, pyrazinylmethyl, isoquinolinylmethyl, pyridylethyl and thienylethyl.

In the aralkyl (arylalkyl) groups and heteroaralkyl (heteroarylalkyl), and heteroalkyl groups, “alkyl” refers to a divalent alkylene group preferably having 1 to 4 carbon atoms.

Carbocyclic structures are non-aromatic monocyclic or bicyclic structures containing 5 to 14 carbon atoms, preferably 6 to 10 carbon atoms, wherein the ring structure(s) optionally contain at least one C═C bond.

Dialkylamino means two alkyl groups as defined herein attached through a nitrogen atom linkage.

Substituted radicals preferably have 1 to 3 substituents, especially 1 or 2 substituents.

According to a compound and/or method aspect of the present invention, the compounds are selected from:
(1) 3-(hexahydropyrrolo[1,2-a]pyrazin-2(1H)-yl)-1-(phenylsulfonyl)-1H-pyrrolo[3,2-b]pyridine hydroformate.
(2) 1-[(3-chlorophenyl)sulfonyl]-3-(hexahydropyrrolo[1,2-a]pyrazin-2(1H)-yl)-1H-pyrrolo[3,2-b]pyridine hydroformate.
(3) 1-[(2-chlorophenyl)sulfonyl]-3-(hexahydropyrrolo[1,2-a]pyrazin-2(1H)-yl)-1H-pyrrolo[3,2-b]pyridine hydroformate.
(4) 1-[(3-fluorophenyl)sulfonyl]-3-(hexahydropyrrolo[1,2-a]pyrazin-2(1H)-yl)-1H-pyrrolo[3,2-b]pyridine hydroformate.
(5) 1-[(2-fluorophenyl)sulfonyl]-3-(hexahydropyrrolo[1,2-a]pyrazin-2(1H)-yl)-1H-pyrrolo[3,2-b]pyridine hydroformate.
(6) 3-(hexahydropyrrolo[1,2-a]pyrazin-2(1H)-yl)-1-[(3-methoxyphenyl)sulfonyl]-1H-pyrrolo[3,2-b]pyridine hydroformate.
(7) 3-(hexahydropyrrolo[1,2-a]pyrazin-2(1H)-yl)-1-[(2-methoxyphenyl)sulfonyl]-1H-pyrrolo[3,2-b]pyridine hydroformate.
(8) 3-{[3-(hexahydropyrrolo[1,2-a]pyrazin-2(1H)-yl)-1H-pyrrolo[3,2-b]pyridin-1-yl]sulfonyl}benzonitrile hydroformate.
(9) 2-{[3-(hexahydropyrrolo[1,2-a]pyrazin-2(1H)-yl)-1H-pyrrolo[3,2-b]pyridin-1-yl]sulfonyl}-4-methylbenzonitrile hydroformate.
(10) 1-(2,3-dihydro-1-benzofuran-6-ylsulfonyl)-3-(hexahydropyrrolo[1,2-a]pyrazin-2(1H)-yl)-1H-pyrrolo[3,2-b]pyridine hydroformate.
(11) 1-(2,3-dihydro-1-benzofuran-4-ylsulfonyl)-3-(hexahydropyrrolo[1,2-a]pyrazin-2(1H)-yl)-1H-pyrrolo[3,2-b]pyridine hydroformate.
(12) 2-[1-(phenylsulfonyl)-1H-pyrrolo[3,2-b]pyridin-3-yl]octahydro-2H-pyrido[1,2-a]pyrazine hydroformate.
(13) 2-{1-[(2-chlorophenyl)sulfonyl]-1H-pyrrolo[3,2-b]pyridin-3-yl}octahydro-2H-pyrido[1,2-a]pyrazine hydroformate.
(14) 2-{1-[(3-chlorophenyl)sulfonyl]-1H-pyrrolo[3,2-b]pyridin-3-yl}octahydro-2H-pyrido[1,2-a]pyrazine hydroformate.
(15) 2-{1-[(4-chlorophenyl)sulfonyl]-1H-pyrrolo[3,2-b]pyridin-3-yl}octahydro-2H-pyrido[1,2-a]pyrazine hydroformate.
(16) 2-[1-(pyridin-3-ylsulfonyl)-1H-pyrrolo[3,2-b]pyridin-3-yl]octahydro-2H-pyrido[1,2-a]pyrazine hydroformate.
(17) 2-{1-[(3-fluorophenyl)sulfonyl]-1H-pyrrolo[3,2-b]pyridin-3-yl}octahydro-2H-pyrido[1,2-a]pyrazine hydroformate.

Wherein the compounds listed above can also be in the form of a free base or another pharmaceutically acceptable salt,

wherein a compound listed above can also be in the form of a solvate (such as a hydrate) or a solvate of a salt,

wherein a compound listed above (in a free base form or solvate thereof, or in the form of a pharmaceutically acceptable salt or solvate thereof) can also be in the form of a polymorph, and

wherein if the compound exhibits chirality it can be in the form of a mixture of enantiomers such as a racemate or a mixture of diastereomers, or can be in the form of a single enantiomer or a single diastereomer.

The following table presents structures for selected compounds of the present invention:

| Com- | | LC-MS (m/z; t_{R}) |
|---|---|---|
| pound | Structure | Method) |
| (1) | 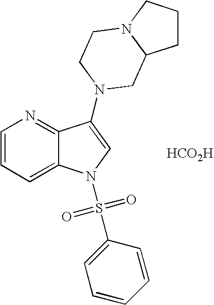 | [M + 1]⁺ 383 at 3.83 min (Analytical Method A) |
| (2) | 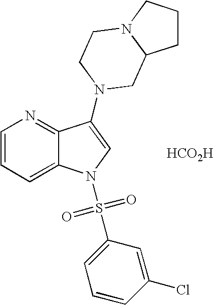 | [M + 1]⁺ 417 at 4.14 min (Analytical Method A) |
| (3) | 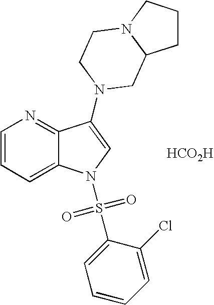 | [M + 1]⁺ 417 at 3.99 min (Analytical Method A) |
| (4) | 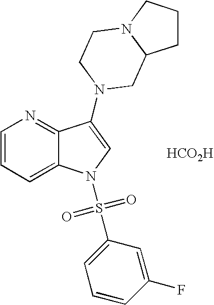 | [M + 1]⁺ 401.1 at 5.36 min (Analytical Method C) |
| (5) | 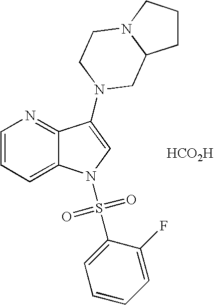 | [M + 1]⁺ 401 at 3.92 min (Analytical Method A) |
| (6) | 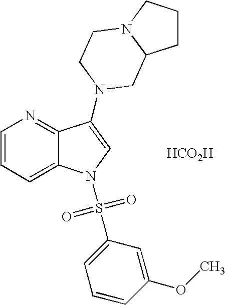 | [M + 1]⁺ 413 at 4.01 min (Analytical Method A) |
| (7) | 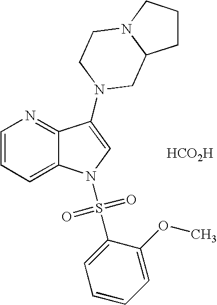 | [M + 1]⁺ 413 at 3.86 min (Analytical Method A) |
| (8) | 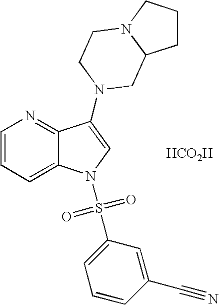 | [M + 1]⁺ 408 at 3.86 min (Analytical Method A) |
| (9) | 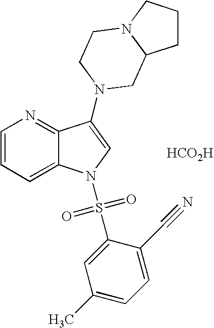 | [M + 1]⁺ 422 at 4.01 min (Analytical Method A) |
| (10) | 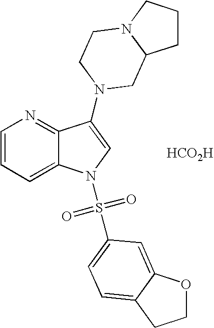 | [M + 1]⁺ 425 at 4.04 min (Analytical Method A) |
| (11) | 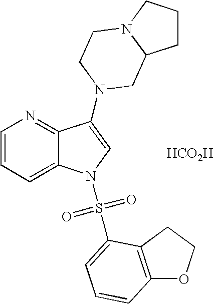 | [M + 1]⁺ 425 at 3.16 min (Analytical Method A) |
| (12) | 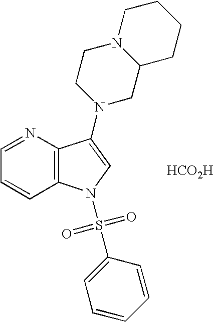 | [M + 1]⁺ 397 at 3.95 min (Analytical Method A) |
| (13) | 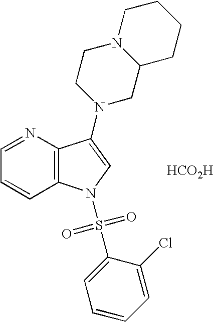 | [M + 1]⁺ 431 at 4.03 min (Analytical Method A) |
| (14) | 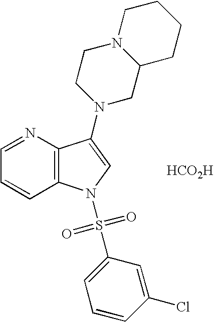 | [M + 1]⁺ 431 at 4.22 min (Analytical Method A) |
| (15) | 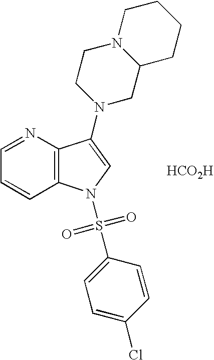 | [M + 1]⁺ 431 at 4.20 min (Analytical Method A) |
| (16) | 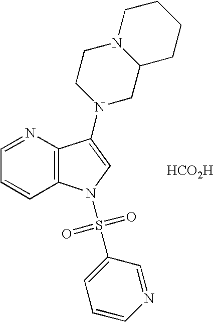 | [M + 1]⁺ 398.1 at 3.62 min (Analytical Method C) |
| (17) | 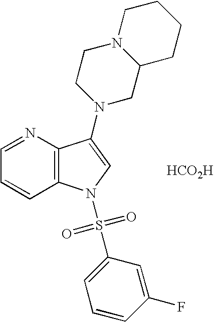 | [M + 1]⁺ 415.0 at 4.17 min (Analytical Method C) |

Analytical HPLC was performed on a 4.6 mm×100 mm Waters Sunfire RP C18 5 mm column using a gradient of typically (i) 20/80 to 80/20 acetonitrile (0.1% formic acid)/water (0.1% formic acid) over 8 min (Analytical Method A); (ii) 10/90 to 80/20 acetonitrile (0.1% formic acid)/water (0.1% formic acid) over 8 min (Analytical Method B); or (iii) 05/95 to 60/40 acetonitrile (0.1% formic acid)/water (0.1% formic acid) over 8 min (Analytical Method C).

Also contemplated for each of the free bases in Table 1 is a pharmaceutically acceptable salt thereof. For each of the salts listed in Table 1, the invention also contemplates the free base of the salt as well as a different pharmaceutically acceptable salt thereof.

Additional aspects of the present invention include pharmaceutical compositions comprising a compound of this invention and a pharmaceutically acceptable carrier and, optionally, one or more additional active agent(s) as discussed below. Further aspects include methods of treating a disease state related to or modulated by the 5-HT₆ receptor, in a patient, such as a mammal, e.g., a human, e.g., those disease states mentioned herein.

In one embodiment, the compounds are selective antagonists or partial antagonists of the 5-HT₆ receptor. These compounds are particularly useful for treating states associated with CNS disorders, motor, mood, personality, behavioral, psychiatric, cognitive, and neurodegenerative disorders, disorders associated with spinal trauma and/or head injury, memory/cognitive impairment, and gastrointestinal (GI) disorders.

In some embodiments, the compounds of the present invention are effective as agonists of the 5-HT₆ receptor. These compounds exhibit activity, especially where such activity affects states associated with depression and any disease or impairment associated with decreased extracellular GABA concentrations or increased glutamate release caused by ischemic-inducing agents.

All methods comprise administering to the patient in need of such treatment an effective amount of one or more compounds of the invention.

A subject or patient in whom administration of the therapeutic compound is an effective therapeutic regimen for a disease or disorder is preferably a human, but can be any animal, including a laboratory animal in the context of a clinical trial or screening or activity experiment. Thus, as can be readily appreciated by one of ordinary skill in the art, the methods, compounds and compositions of the present invention are particularly suited to administration to any animal, particularly a mammal, and including, but by no means limited to, humans, domestic animals, such as feline or canine subjects, farm animals, such as but not limited to bovine, equine, caprine, ovine, and porcine subjects, wild animals (whether in the wild or in a zoological garden), research animals, such as mice, rats, rabbits, goats, sheep, pigs, dogs, cats, etc., avian species, such as chickens, turkeys, songbirds, etc., e.g., for veterinary medical use.

The compounds of the present invention may be prepared using conventional synthetic methods analogous to those established in the art, and, if required, standard separation or isolation techniques. Suitable synthetic procedures that may be used to prepare the compounds of the present invention are described in, for example, U.S. Pat. Nos. 6,133,217, 6,191,141, and 6,903,112. All starting materials are either commercially available, or can be conventionally prepared from known starting materials without undue experimentation.

One of ordinary skill in the art will recognize that some of the compounds of Formula I can exist in different geometrical isomeric forms. In addition, some of the compounds of the present invention possess one or more asymmetric atoms and are thus capable of existing in the form of optical isomers, as well as in the form of racemic or nonracemic mixtures thereof, and in the form of diastereomers and diastereomeric mixtures inter alia. All of these compounds, including cis isomers, trans isomers, diastereomeric mixtures, racemates, nonracemic mixtures of enantiomers, substantially pure, and pure enantiomers, are within the scope of the present invention. In one embodiment, substantially pure enantiomers contain no more than 5% w/w of the corresponding opposite enantiomer, preferably no more than 2%, most preferably no more than 1%.

The optical isomers can be obtained by resolution of the racemic mixtures according to conventional processes, for example, by the formation of diastereomeric salts using an optically active acid or base or formation of covalent diastereomers.

Examples of appropriate acids include, but are not limited to, tartaric, diacetyltartaric, dibenzoyltartaric, ditoluoyltartaric and camphorsulfonic acid. Mixtures of diastereomers can be separated into their individual diastereomers on the basis of their physical and/or chemical differences by methods known to those skilled in the art, for example, by chromatography or fractional crystallization. The optically active bases or acids are then liberated from the separated diastereomeric salts.

A different process for separation of optical isomers involves the use of chiral chromatography (e.g., chiral HPLC or SFC columns), with or without conventional derivation, optimally chosen to maximize the separation of the enantiomers. Suitable chiral HPLC columns are manufactured by Diacel, e.g., Chiracel OD and Chiracel OJ among many others, all routinely selectable. Enzymatic separations, with or without derivatization, are also useful. The optically active compounds of Formulas I-II can likewise be obtained by utilizing optically active starting materials in chiral syntheses processes under reaction conditions which do not cause racemization.

In addition, one of ordinary skill in the art will recognize that the compounds can be used in different enriched isotopic forms, e.g., enriched in the content of ²H, ³H, ¹¹C, ¹³C and/or ¹⁴C. In one particular embodiment, the compounds are deuterated. Such deuterated forms can be made by the procedure described in U.S. Pat. Nos. 5,846,514 and 6,334,997. As described in U.S. Pat. Nos. 5,846,514 and 6,334,997, deuteration can improve the efficacy and increase the duration of action of drugs.

Deuterium substituted compounds can be synthesized using various methods such as

described in: Dean, Dennis C.; Editor. Recent Advances in the Synthesis and Applications of Radiolabeled Compounds for Drug Discovery and Development. [In: Curr., Pharm. Des., 2000; 6(10)] (2000), 110 pp. CAN 133:68895 AN 2000:473538 CAPLUS; Kabalka, George W., Varma, Rajender S. The Synthesis of Radiolabeled Compounds via Organometallic Intermediates. Tetrahedron (1989), 45(21), 6601-21, CODEN: TETRAB ISSN:0040-4020. CAN 112:20527 AN 1990:20527 CAPLUS; and Evans, E. Anthony. Synthesis of radiolabeled compounds, J. Radioanal. Chem. (1981), 64(1-2), 9-32. CODEN: JRACBN ISSN:0022-4081, CAN 95:76229 AN 1981:476229 CAPLUS.

The present invention also relates to useful forms of the compounds as disclosed herein, including free base forms, as well as pharmaceutically acceptable salts or prodrugs of all the compounds of the present invention for which salts or prodrugs can be prepared. Pharmaceutically acceptable salts include those obtained by reacting the main compound, functioning as a base, with an inorganic or organic acid to form a salt, for example, but not limited to, salts of hydrochloric acid, sulfuric acid, phosphoric acid, methanesulfonic acid, camphorsulfonic acid, oxalic acid, maleic acid, succinic acid and citric acid. Pharmaceutically acceptable salts also include those in which the main compound functions as an acid and is reacted with an appropriate base to form, e.g., sodium, potassium, calcium, magnesium, ammonium, and choline salts. Those skilled in the art will further recognize that acid addition salts of the claimed compounds may be prepared by reaction of the compounds with the appropriate inorganic or organic acid via any of a number of known methods. Alternatively, alkali and alkaline earth metal salts are prepared by reacting the compounds of the invention with the appropriate base via a variety of known methods.

The following are further non-limiting examples of acid salts that can be obtained by reaction with inorganic or organic acids: acetates, adipates, alginates, citrates, aspartates, benzoates, benzenesulfonates, bisulfates, butyrates, camphorates, digluconates, cyclopentanepropionates, dodecylsulfates, ethanesulfonates, glucoheptanoates, glycerophosphates, hemisulfates, heptanoates, hexanoates, fumarates, hydrobromides, hydroiodides, 2-hydroxy-ethanesulfonates, lactates, maleates, methanesulfonates, nicotinates, 2-naphthalenesulfonates, oxalates, palmoates, pectinates, persulfates, 3-phenylpropionates, picrates, pivalates, propionates, succinates, tartrates, thiocyanates, tosylates, mesylates and undecanoates.

For example, the pharmaceutically acceptable salt can be a hydrochloride, hydroformate, hydrobromide, or maleate.

Preferably, the salts formed are pharmaceutically acceptable for administration to mammals. However, pharmaceutically unacceptable salts of the compounds are suitable as intermediates, for example, for isolating the compound as a salt and then converting the salt back to the free base compound by treatment with an alkaline reagent. The free base can then, if desired, be converted to a pharmaceutically acceptable acid addition salt.

One of ordinary skill in the art will also recognize that some of the compounds of Formula I can exist in different polymorphic forms. As known in the art, polymorphism is an ability of a compound to crystallize as more than one distinct crystalline or “polymorphic” species. A polymorph is a solid crystalline phase of a compound with at least two different arrangements or polymorphic forms of that compound molecule in the solid state. Polymorphic forms of any given compound are defined by the same chemical formula or composition and are as distinct in chemical structure as crystalline structures of two different chemical compounds.

One of ordinary skill in the art will further recognize that compounds of Formula I can exist in different solvate forms. Solvates of the compounds of the invention may also form when solvent molecules are incorporated into the crystalline lattice structure of the compound molecule during the crystallization process. For example, suitable solvates include hydrates, e.g., monohydrates, dihydrates, sesquihydrates, and hemihydrates.

The compounds of the invention can be administered alone or as an active ingredient of a formulation. Thus, the present invention also includes pharmaceutical compositions of one or more compounds of Formula I containing, for example, one or more pharmaceutically acceptable carriers.

Numerous standard references are available that describe procedures for preparing various formulations suitable for administering the compounds according to the invention. Examples of potential formulations and preparations are contained, for example, in the Handbook of Pharmaceutical Excipients, American Pharmaceutical Association (current edition); Pharmaceutical Dosage Forms: Tablets (Lieberman, Lachman and Schwartz, editors) current edition, published by Marcel Dekker, Inc., as well as Remington's Pharmaceutical Sciences (Arthur Osol, editor), 1553-1593 (current edition).

In view of their high degree of selective 5-HT₆ receptor activity, the compounds of the present invention can be administered to anyone requiring modulation of the 5-HT₆ receptor. Administration may be accomplished according to patient needs, for example, orally, nasally, parenterally (subcutaneously, intravenously, intramuscularly, intrasternally and by infusion) by inhalation, rectally, vaginally, topically and by ocular administration.

Various solid oral dosage forms can be used for administering compounds of the invention including such solid forms as tablets, gelcaps, capsules, caplets, granules, lozenges and bulk powders. The compounds of the present invention can be administered alone or combined with various pharmaceutically acceptable carriers, diluents (such as sucrose, mannitol, lactose, starches) and excipients known in the art, including but not limited to suspending agents, solubilizers, buffering agents, binders, disintegrants, preservatives, colorants, flavorants, lubricants and the like. Time release capsules, tablets and gels are also advantageous in administering the compounds of the present invention.

Various liquid oral dosage forms can also be used for administering compounds of the inventions, including aqueous and non-aqueous solutions, emulsions, suspensions, syrups, and elixirs. Such dosage forms can also contain suitable inert diluents known in the art such as water and suitable excipients known in the art such as preservatives, wetting agents, sweeteners, flavorants, as well as agents for emulsifying and/or suspending the compounds of the invention. The compounds of the present invention may be injected, for example, intravenously, in the form of an isotonic sterile solution. Other preparations are also possible.

Suppositories for rectal administration of the compounds of the present invention can be prepared by mixing the compound with a suitable excipient such as cocoa butter, salicylates and polyethylene glycols. Formulations for vaginal administration can be in the form of a pessary, tampon, cream, gel, paste, foam, or spray formula containing, in addition to the active ingredient, such suitable carriers as are known in the art.

For topical administration, the pharmaceutical composition can be in the form of creams, ointments, liniments, lotions, emulsions, suspensions, gels, solutions, pastes, powders, sprays, and drops suitable for administration to the skin, eye, ear or nose. Topical administration may also involve transdermal administration via means such as transdermal patches.

Aerosol formulations suitable for administering via inhalation also can be made. For example, for treatment of disorders of the respiratory tract, the compounds according to the invention can be administered by inhalation in the form of a powder (e.g., micronized) or in the form of atomized solutions or suspensions. The aerosol formulation can be placed into a pressurized acceptable propellant.

Assays for determining 5-HT₆ receptor activity, and selectivity of 5-HT₆ receptor activity are known within the art. See, for example, U.S. Pat. Nos. 6,133,287, 6,686,374, and 6,903,112, and Example 8 described below. Compounds of the invention show 5-HT₆ binding activity with receptor Ki values of typically less than 1-100 nM. In one embodiment, the binding activity will be less than 1-50 nM, and in another embodiment, the activity will be less than 1-10 nM. Compounds of the invention show 5-HT₆ functional activity with pA2 values of greater than 6 (IC₅₀ less than 1 μM). In one embodiment, the pA2 value will be greater than 7 (IC₅₀ less than 500 nM), and in another embodiment, the pA2 value will be greater than 8 (IC₅₀ less than 100 nM).

A pharmacokinetic profile of the compounds may be further shown with measurements to determine hERG and Cyp3A4 inhibition. The hERG inhibition may be measured as described by Dubin, A. (2004). HERG Potassium Channel Activity Assayed with the PatchXpress Planar Patch Clamp. Inaugural PatchXpress Users Meeting, Feb. 12, 2004 (Baltimore, Md.). The Cyp inhibition may be measured as described by Miller V P, Stresser D M, Blanchard A P, Turner S, Crespi C L: Fluorometric high-throughput screening for inhibitors of cytochrome P450. Ann N Y Acad Sci 200; 919:26-32. In one embodiment, the compounds show HERG inhibition with an IC₅₀ greater than 1 μM; in another embodiment, the hERG inhibition is greater than 3 μM, and in yet another embodiment, it is greater than 10 μM, In another embodiment, the compounds show Cyp3A4 inhibition with an IC₅₀ greater than 1 μM, which may be greater than 3 μM, and, in another embodiment, it is greater than 10 μM.

High hERG inhibition and Cyp3A4 inhibition is potentially linked with adverse cardiac action potential and drug metabolism, respectively.

According to a method aspect, the invention includes a method for the treatment of a disorder of the central nervous system (CNS) related to or affected by the 5-HT₆ receptor in a patient in need thereof by administering to the patient a therapeutically effective amount of a compound selected from formula I, as described herein above. The compounds can be administered as the sole active agent or in combination with other pharmaceutical agents.

The compounds of the present invention are effective in inhibiting, or modulating the activity of the 5-HT₆ receptor in animals, e.g., mammals, especially humans. These compounds are used for the treatment and/or prophylaxis of a disease or disorders related to or affected by the 5-HT₆ receptor. These compounds are used for the treatment and/or prophylaxis of a disease or disorders associated with the 5-HT₆ receptor. The compounds may be antagonists, partial antagonists, agonists, or partial agonists. These compounds exhibit activity, especially where such activity affects states associated with CNS disorders including motor, mood, personality, behavioral, psychiatric, cognitive, and neurodegenerative disorders, such as, but not limited to, Alzheimer's disease (enhancement of cognitive memory), Parkinson's disease, Huntington's disease, anxiety, depression, manic depression, epilepsy, obsessive compulsive disorders, migraine, sleep disorders, feeding disorders such as anorexia and bulimia, panic attacks, attention deficit hyperactivity disorder (ADHD), attention deficit disorder (ADD), amyotrophic lateral sclerosis, AIDS dementia, retinal diseases, withdrawal from drug abuse such as cocaine, ethanol, nicotine and benzodiazepines, psychoses, such as schizophrenia, bipolar disorder.

The compounds are also effective for treating psychotic disorders. Such psychotic disorders include schizophrenia, late-onset schizophrenia, schizoaffective disorders, prodromal schizophrenia, bipolar disorders, psychoses resulting from drug abuse, post-traumatic stress disorder (PTSD), and schizoid personality.

Psychoses are disorders that affect an individual's perception of reality. Psychoses are characterized by delusions and hallucinations. The present invention includes methods for treating patients suffering from all forms of psychoses, including but not limited to schizophrenia, late-onset schizophrenia, schizoaffective disorders, prodromal schizophrenia, and bipolar disorders. Treatment may be for the positive symptoms of schizophrenia as well as for the cognitive deficits and negative symptoms. Other indications for 5-HT₆ ligands include psychoses resulting from drug abuse (including amphetamines and PCP), encephalitis, alcoholism, epilepsy, Lupus, sarcoidosis, brain tumors, multiple sclerosis, dementia with Lewy bodies, or hypoglycemia. Other psychiatric disorders, like posttraumatic stress disorder (PTSD), and schizoid personality may also be treated with 5-HT₆ ligands.

The compounds are also effective for treating disorders associated with spinal trauma and/or head injury such as hydrocephalus. Such acute neurodegenerative disorders also include strokes, such as acute thromboembolic strokes, focal and global ischemia, transient cerebral ischemic attacks or other cerebral vascular problems accompanied by cerebral ischemia, fetal hypoxia, hypoglycemia, hypotension, injuries from procedures for embole, hyperfusion or hypoxia and asphyxia

The compounds are also effective for treating a patient undergoing a procedure such as surgery, or more particularly cardiac surgery, in incidents of cranial hemorrhage, in perinatal asphyxia, in cardiac arrest, status epilepticus, post-operative surgery (CABG) or other incidents, especially where blood flow to the brain is halted for a period of time.

The compounds of the present invention are useful for treating dementias. Dementias that may be treated include those caused by a neurodegenerative disease or disorder (i.e, alzheimer's disease, Parkinson's disease, Huntington's disease, Pick's disease), a vascular disease or disorder (e.g., infarcts, hemorrhage, cardiac disorders), a traumatic injury (e.g., subdural hematoma, traumatic brain injury), an infectious disease or disorder (e.g., HIV), a genetic disease or disorder (e.g., Down syndrome), toxicity (e.g., exposure to heavy metals, alcohol, medications, a metabolic disease or disorder (e.g., B12 or foliate deficiency), a psychiatric disease or disorder (e.g., depression schizophrenia), or dementias arising from other causes (e.g., mixed vascular and alzheimer's disease, bacterial meningitis, Creutzfeld-Jakob, multiple sclerosis, CNS hypoxia, Cushing's disease, and hydrocephalus.

Dementias are diseases that include memory loss and additional intellectual impairment separate from memory. The present invention includes methods for treating patients suffering from memory impairment in all forms of dementia. Dementias are classified according to their cause and include: neurodegenerative dementias (e.g., Alzheimer's, Parkinson's disease, Huntington's disease, Pick's disease), vascular (e.g., infarcts, hemorrhage, cardiac disorders), mixed vascular and Alzheimer's, bacterial meningitis, Creutzfeld-Jacob Disease, multiple sclerosis, traumatic (e.g., subdural hematoma or traumatic brain injury), infectious (e.g., HIV), genetic (Down syndrome), toxic (e.g., heavy metals, alcohol, some medications), metabolic (e.g., vitamin B12 or folate deficiency), CNS hypoxia, Cushing's disease, psychiatric (e.g., depression and schizophrenia), and hydrocephalus.

Such compounds are also useful for the treatment of memory/cognitive impairment associated with Alzheimer's disease, schizophrenia, Parkinson's disease, Huntington's disease Pick's disease, Creutzfeld Jakob disease, HIV, cardiovascular disease, head trauma, age-related cognitive decline, depression, aging, use of general anesthetics, age-related cognitive decline, head trauma, stroke, schizophrenia, spinal cord injury, CNS hypoxia, cerebral senility, diabetes associated cognitive impairment, memory deficits from early exposure of anesthetic agents, multiinfarct dementia, other neurological conditions including acute neuronal diseases, HIV, cardiovascular diseases, memory disorders associated with bipolar disorders, and chemotherapy-induced memory loss

The condition of memory impairment is manifested by impairment of the ability to learn new information and/or die inability to recall previously learned information. The present invention includes methods for dealing with memory loss separate from dementia, including mild cognitive impairment (MCI) and age-related cognitive decline. The present invention includes methods of treatment for memory impairment as a result of disease. Memory impairment is a primary symptom of dementia and can also be a symptom associated with such diseases as Alzheimer's disease, schizophrenia, Parkinson's disease, Huntington's disease, Pick's disease, Creutzfeld-Jakob disease, HIV, cardiovascular disease, and head trauma as well as age-related cognitive decline. In another application, the invention includes methods for dealing with memory loss resulting from the use of general anesthetics, chemotherapy, radiation treatment, post-surgical trauma, and therapeutic intervention. Thus, in accordance with one embodiment, the present invention includes methods of treating patients suffering from memory impairment due to, for example, Alzheimer's disease, multiple sclerosis, amylolateroscierosis (ALS), multiple systems atrophy (MSA), schizophrenia, Parkinson's disease, Huntington's disease, Pick's disease, Creutzfeld-Jakob disease, depression, aging, head trauma, stroke, spinal cord injury, CNS hypoxia, cerebral senility, diabetes associated cognitive impairment, memory deficits from early exposure of anesthetic agents, multiinfarct dementia and other neurological conditions including acute neuronal diseases, as well as HIV and cardiovascular diseases. The invention also relates to agents and/or methods to stimulate the formation of memory in “normal” subjects (i.e., subjects who do not exhibit an abnormal or pathological decrease in a memory function), e.g., ageing middle-aged subjects.

Compounds of the present invention are useful for the treatment of polyglutamine-repeat diseases such as Huntington's disease, dentatorubral-pallidoluysian atrophy (DRPLA), spinocerebellar ataxia type-1 spinocerebellar ataxia type-2 (ataxin-2), spinocerebellar ataxia type-3 (ataxin-3) Machado-Joseph disease, (MJD), spinocerebellar ataxia type-6 (ataxin-6), spinocerebellar ataxia type-7 (ataxin-7), and spinal and bulbar muscular atrophy (SMBA), also known as Kennedy's disease, (androgen receptor).

The invention is also suitable for use in the treatment of a class of disorders known as polyglutamine-repeat diseases. These diseases share a common pathogenic mutation. The expansion of a CAG repeat, which encodes the amino acid glutamine, within the genome leads to production of a mutant protein having an expanded polyglutamine region. For example, Huntington's disease has been linked to a mutation of the protein huntingtin. In individuals who do not have Huntington's disease, huntingtin has a polyglutamine region containing about 8 to 31 glutamine residues. For individuals who have Huntington's disease, huntingtin has a polyglutamine region with over 37 glutamine residues. Aside from Huntington's disease (HD), other known polyglutamine-repeat diseases and the associated proteins are: dentatorubral-pallidoluysian atrophy, DRPLA (atrophin-1); spinocerebellar ataxia type-1 (ataxin-1); spinocerebellar ataxia type-2 (ataxin-2); spinocerebellar ataxia type-3 also called Macliado-Joseph disease, MJD (ataxin-3); spinocerebellar ataxia type-6 (alpha 1a-voltage dependent calcium channel); spinocerebellar ataxia type-7 (ataxin-7); and spinal and bulbar muscular atrophy, SBMA, also known as Kennedy disease (androgen receptor). Thus, in accordance with a further aspect of the invention, there is provided a method of treating a polyglutamine-repeat disease or CAG repeat expansion disease comprising administering to a patient, such as a mammal, especially a human, a therapeutically effective amount of a compound. In accordance with a further embodiment, there is provided a method of treating Huntington's disease (HD), dentatorubral-pallidoluysian atrophy (DRPLA), spinocerebellar ataxia type-1, spinocerebellar ataxia type-2, spinocerebellar ataxia type-3 (Machado-Joseph disease), spinocerebellar ataxia type-6, spinocerebellar ataxia type-7, or spinal and bulbar muscular atrophy, comprising administering to a patient, such as a mammal, especially a human, a therapeutically effective amount of a compound of the invention.

Compounds of the present invention are useful for the treatment of movement disorders related to dysfunction of basal ganglia neurons, prefrontal cortex and hippocampus, including tpsychoses, Parkinson's disease, progressive supranuclear palsy, cerebral palsy, coritcobasal degeneration, multiple system atrophy, Wilson disease, dystonia, tics, dementias, obsessive compulsion disorder, tardive dyskinesia, choreas, depression, mood disorders, impulsivity, drug addiction, attention deficit/hyperactivity disorder (ADHD), depression with Parkinsonian states, personality changes with caudate or putamen disease, dementia and mania with caudate and pallidal diseases, compulsions with pallidal disease.

Such compounds are also expected to be of use in the treatment of certain gastrointestinal (GI) disorders such as, but not limited to, functional bowel disorder, constipation, including chronic constipation, gastroesophageal reflux disease (GERD), nocturnal-GERD, and irritable bowel syndrome (IBS), including diarrhea-predominant IBS (IBS-c), constipation-predominant IBS (IBS-c) and alternating constipation/diarrhea IBS. See for example, B. L. Roth et al., J. Pharmacol. Exp. Ther., 1994, 268, pages 1403-14120, D. R. Sibley et al., Mol. Pharmacol., 1993, 43, 320-327, A. J. Sleight et al., Neurotransmission, 1995, 11, 1-5, and A. J. Sleight et al. Serotonin ID Research Alert, 1997, 2 (3), 115-8). Furthermore, the effect of 5-HT₆ antagonist and 5-HT₆ antisense oligonucleotides to reduce food intake in rats has been reported (Br. J. Pharmac., 1999 Suppl. 126, page 66 and J. Psychopharmacol Suppl. A64, 1997, page 255.

The compounds are also effective for treating inflammatory diseases such as ulcerative colitis, fibromyalgia, and autoimmune diseases.

Indications that may be treated with 5-HT₆ ligands, either alone or in combination with other drugs, include, but are not limited to, those diseases thought to be mediated in part by the basal ganglia, prefrontal cortex and hippocampus. These indications include psychoses, Parkinson's disease, dementias, obsessive compulsion disorder, tardive dyskinesia, choreas, depression, mood disorders, impulsivity, drug addiction, attention deficit/hyperactivity disorder (ADHD), depression with parkinsonian states, personality changes with caudate or putamen disease, dementia and mania with caudate and pallidal diseases, and compulsions with pallidal disease.

The basal ganglia are important for regulating the function of motor neurons; disorders of the basal ganglia result in movement disorders. Most prominent among the movement disorders related to basal ganglia function is Parkinson's disease (Obeso J A et al., Neurology., 2004 Jan. 13; 62(1 Suppl 1):S17-30). Other movement disorders related to dysfunction of the basla ganglia include tardive dyskinesia, progressive supranuclear palsy and cerebral palsy, corticobasal degeneration, multiple system atrophy, Wilson disease, and dystonia, tics, and chorea. In one embodiment, the compounds of the invention may be used to treat movement disorders related to dysfunction of basal ganglia neurons.

Another aspect of the invention includes methods for treating attention deficit hyperactivity disorder (ADHD) and/or attention deficit disorder (ADD) comprising administering to a patient, simultaneously or sequentially, the compound of the invention and one or more additional agents used in the treatment of ADHD and/or ADD, such as, but not limited to amphetamine/dextroamphetamine (Adderall); atomoxetine (Strattera); bupropion (Wellbutrin, Budeprion); dexmethylphenidate (Focalin); dextroamphetamine (Dexedrine, Spansules, Dextrostat); lisdexamfetamine (Vyvanse); methamphetamine (Desoxyn); methylphenidate (Concerta, Ritalin, Daytrana, Metadate, Methylin); and pemoline (Cylert). In methods using simultaneous administration, the agents can be present in a combined composition or can be administered separately. As a result, the invention also includes compositions comprising a compound according to Formula I and one or more additional pharmaceutical agents used in the treatment of ADIID and/or ADD such as, but not limited to, amphetamine/dextroamphetamine (Adderall); atomoxetine (Strattera); bupropion (Wellbutrin, Budeprion); dexmethylphenidate (Focalin); dextroaniphetamine (Dexedrine, Spansules, Dextrostat); lisdexamfetamine (Vyvanse); methamphetamine (Desoxyn); methylphenidate (Concerta, Ritalin, Daytrana, Metadate, Methylin); and pemoline (Cylert). Similarly, the invention also includes kits containing a composition comprising a compound according to Formula I and another composition useful for treating ADHD and/or ADD.

Yet another aspect of the invention includes methods for treating obesity. Obesity and the regulation of food intake (e.g., weight control) can be regulated or treated with the compounds of the present invention, since 5-HT₆ plays an important part in within-meal satisfaction and post-meal satisfaction processes as well as other processes for weight regulation. Thus, the compounds of formula (I) to decrease food intake when given acutely or chronically can be effectively used to regulate weight. This reduction in weight may also be concomitant to improving a number of cardio-metabolic risk factors. The compounds can be administered in combination with other pharmaceutical agents used in the treatment of obesity or for otherwise regulating food intake, e.g., Diethylpropion (Tenuate); orlistat (Xenical, Alli); phendimetrazines (Bontril, Adipost, Anorex, Appecon, Melfiat, Obezine, Phendiet, Plegine, Prelu-2, Statobex); sibutramine (Meridia); benzphetamine (Didrex); methamphetamine (Desoxyn); metformin; Byetta; Symlin; dexfenfluramine; fluoxetine; chlorophenylpiperazine; and Rimonabant. Thus, the invention also includes methods for treating or affecting obesity comprising administering to a patient, simultaneously or sequentially, the compound of the invention and one or more additional agents used in the treatment of obesity such as, but not limited to, Diethylpropion (Tenuate); orlistat (Xenical, Alli); phendimetrazines (Bontril, Adipost, Anorex, Appecon, Melfiat, Obezine, Phendiet, Plegine, Prelu-2, Statobex), sibutramine (Meridia); benzphetamine (Didrex); methamphetamine (Desoxyn); metformin; Byetta; Symlin; dexfenfluramine; fluoxetine; chlorophenylpiperazine; and Rimonabant.

In addition, such compounds are expected to be useful for encephalitis, alcoholism, epilepsy, Lupus, sarcoidosis, brain tumors, multiple sclerosis, dementia with Lewy bodies, and hypoglycemia, and kidney dialysis.

Other diseases and conditions that may be treated with the compounds as described herein include the diseases and conditions listed on the NIMH list or on the DMS5 list.

In one embodiment, the compounds of the invention can be administered in combination with a nicotinic acetylcholine subtype α-7 receptor ligand (α-7 receptor ligand). Nicotinic acetylcholine subtype α-7 receptor liganids modulate the function of nicotinic acetylcholine subtype α-7 receptors by altering the activity of the receptor. Suitable compounds also can be partial agonists that partially block or partially activate the α-7 receptor or agonists that activate the receptor. Positive allosteric modulators are compounds that potentiate the receptor response to acetylcholine without themselves triggering receptor activation or desensitization, or either, of the receptor. Nicotinic acetylcholine subtype α7 receptor ligands that can be combined with the 5-HT₆ ligand of the present invention can include full agonists, partial agonists, or positive allosteric modulators.

α-7 receptor ligands typically demonstrate Kᵢ values from about 1 nM to about 10 μM when tested by the [³H]-MLA assay. Many having a binding value (“Kᵢ MLA”) of less than 1 μM. According to one embodiment, [³H]-Cytisine binding values (“Kᵢ Cyt”) of the α-7 receptor ligand range from about 50 nM to greater than 100 μM. According to another embodiment, α-7 receptor ligands have Kᵢ MLA value (as measured by MLA assay in view of the Kᵢ Cyt value as measured by [³H]-cytisine binding, such that in the formula D=Kᵢ Cyt/Kᵢ MLA) of at least 50. For example, compounds typically exhibit greater potency at α-7 receptors compared to α4β2 receptors. Although the MLA and [³H]-cytisine binding assays are well known, further details for carrying out the assays are provided in International Publication Nos. WO 2005/028477; WO 2005/066168; US 20050137184; US20050137204; US20050245531; WO 2005/066166; WO 2005/066167; and WO 2005/077899.

Positive allosteric modulators, at concentrations ranging from 1 nM to 10 μM, enhance responses of acetylcholine at α-7 nicotinic receptors expressed endogenously in neurons or cell lines, or via expression of recombinant protein in Xenopus oocytes or in cell lines. α-7 receptor ligands can be used to improve efficacy of 5-HT₆ ligands without exaggerating the side effect profile of such agents.

Accordingly, α-7 receptor ligands that may be combined with the 5-HT₆ ligand can be compounds of various chemical classes. Particularly, some examples of α-7 receptor ligands suitable for the invention include, but are not limited to, diazabicycloalkane derivatives, for example as described in International Publication No. WO 2005/028477; spirocyclic quinuclidinic ether derivatives, for example as described in International Publication No. WO 2005/066168; fused bicycloheterocycle substituted quinuclidine derivatives, for example as described in US Publication Nos. US20050137184; US20050137204; and US20050245531; 3-quinuclidinyl aminosubstituted biaryl derivatives, for example as described in International Publication No. WO 2005/066166; 3-quinuclidinyl heteroatom-bridged biaryl derivatives, for example as described in International Publication No. WO 2005/066167; and aminosubstituted tricyclic derivatives, for example as described in International Publication No. WO 2005/077899, all of which are hereby incorporated by reference in their entirety.

Examples of compounds reported as α-7 agonists or partial agonists are quinuclidine derivatives, for example as described in WO 2004/016608 and WO 2004/022556; and tilorone derivatives, for example also as described in WO 2004/016608.

Examples of compounds reported as positive allosteric modulators are 5-hydroxyindole analogs, for example as described in WO 01/32619, WO 01/32620, and WO 01/32622; tetrahydroquinoline derivatives, for examples as described in WO 04/098600; amino-thiazole derivatives; and diarylurea derivatives, for example as described in WO 04/085433.

Specific examples of compounds that are suitable neuronal nicotinic subtype α-7 receptor ligands include, for example, 5-(6-[(3R)-1-azabicyclo[2.2.2]oct-3-yloxy]pyridazin-3-yl)-1H-indole; 2-(6-phenylpyridazine-3-yl)octahydropyrrolo[3,4-c]pyrrole; 5-[5-{(1R,5R)-6-methyl-3,6-diaza-bicyclo[3.2.0]hept-3-yl}-pyridin-2-yl]-1H-indole; and 5-[6-(cis-5-methyl-hexahydropyrrolo[3,4-c]pyrrol-2-yl)-pyridazin-3-yl-1H-indole. Other suitable α-7 ligands are described in WO2006/101745, which is hereby incorporated by reference.

Compounds modulating activity of nicotinic acetylcholine receptor α-7 subtype are suitable for the invention regardless of the manner in which they affect the receptor. Other compounds reported as demonstrating α-7 activity include, but are not limited to, quinuclidine amide derivatives, for example PNU-282987, N-[(3R)-1-azabicyclo[2.2.2]oct-3-yl]-4-chlorobenzamide TC-5619, varanicline, and others as described in WO 04/052894, and MEM-3454. Additional compounds can include, but are not limited to, AR R17779, AZD0328, WB-56203, SSR-180711A, GTS21, and OH-GTS-21, which are all described in the publicly available literature.

Thus, the compounds of the present invention are useful for the preparation of medicaments for the therapeutic and/or prophylactic treatment of a central nervous system (CNS) disorder, a memory/cognitive impairment, withdrawal from drug abuse, psychoses, a gastrointestinal (GI) disorder, or a polyglutamine-repeat disease. In one aspect of the invention, the CNS disorder is Alzheimer's disease, Parkinson's disease, Huntington's disease, anxiety, depression, manic depression, epilepsy, obsessive compulsive disorders, migraine, sleep disorders, feeding disorders such as anorexia and bulimia, panic attacks, attention deficit hyperactivity disorder (ADHD), attention deficit disorder (ADD), withdrawal from drug abuse, psychoses, or disorders associated with spinal trauma and/or head injury; the memory/cognitive impairment is associated with Alzheimer's disease, schizophrenia, Parkinson's disease, Huntington's disease Pick's disease, Creutzfeld Jakob disease, HIV, cardiovascular disease, head trauma or age-related cognitive decline; or the GI disorder is functional bowel disorder, constipation, gastroesophageal reflux disease (GERD), nocturnal-GERD, irritable bowel syndrome (IBS), constipation-predominant IBS (IBSc) or alternating constipation/diarrhea IBS.

In one aspect of the invention, the compounds of the present invention are useful for the preparation of medicaments for the therapeutic and/or prophylactic treatment of Alzheimer's disease, attention deficit disorder (ADD), schizophrenia, or obesity.

The compounds of the present invention may be combined with other agents to treat the diseases and conditions as described hereinabove. Such as other agents are, for example, used in the treatment of CNS disorders, such as psychoses, especially schizophrenia and bipolar disorder, obsessive-compulsive disorder, Parkinson's disease, cognitive impairment and/or memory loss, e.g., nicotinic α-7 agonists, PDE4 inhibitors, PDE10 inhibitors, other 5-HT₆ receptor ligands, calcium channel blockers, muscarinic m1 and m2 modulators, adenosine receptor modulators, ampakines, NMDA-R modulators, mGluR modulators, dopamine modulators, serotonin modulators, cannabinoid modulators, cholinesterase inhibitors (e.g. donepezil, rivastigimine, and glanthanamine), gamma secretase modulators, Beta secretase modulators, MAO-B modulators, kinase inhibitors, 5-HT₆ receptor ligands, α4β2, Histamine H3, 5-HT₄, ADIID drugs, bipolar drugs, mood stabilizers, anti-psychotics (incl PDE10), α7 modulators, anti-depressants, anti-inflammatories (see Critical Thereapeutics list), and GABAnergic drugs. In such combinations, each active ingredient can be administered either in accordance with their usual dosage range or in accordance with a dose below their usual dosage range.

The compounds can be administered in combination with other pharmaceutical agents used in the treatment of schizophrenia, e.g., Clozaril, Zyprexa, Risperidone, and Seroquel. Thus, the invention also includes methods for treating schizophrenia, including memory impairment associated with schizophrenia, comprising administering to a patient, simultaneously or sequentially, the compound of the invention and one or more additional agents used in the treatment of schizophrenia such as, but not limited to, Clozaril, Zyprexa, Risperidone, and Seroquel. In methods using simultaneous administration, the agents can be present in a combined composition or can be administered separately. As a result, the invention also includes compositions comprising a compound according to Formula I and one or more additional pharmaceutical agents used in the treatment of schizophrenia, e.g., Clozaril, Zyprexa, Risperidone, and Seroquel. Similarly, the invention also includes kits containing a composition comprising a compound according to Formula I and another composition comprising one or more additional pharmaceutical agents used in the treatment of schizophrenia, e.g., Clozaril, Zyprexa, Risperidone, and Seroquel.

In addition, the compounds can be administered in combination with other pharmaceutical agents used in the treatment bipolar disorder such as Lithium, Zyprexa, Depakote, and Zyprexa. Thus, the invention also includes methods for treating bipolar disorder, including treating memory and/or cognitive impairment associated with the disease, comprising administering to a patient, simultaneously or sequentially, the compound of the invention and one or more additional agents used in the treatment of bipolar disorder such as, but not limited to, Lithium, Zyprexa, and Depakote. In methods using simultaneous administration, the agents can be present in a combined composition or can be administered separately. As a result, the invention also includes compositions comprising a compound according to Formula I and one or more additional pharmaceutical agents used in the treatment of bipolar disorder such as, but not limited to, Lithium, Zyprexa, and Depakote. Similarly, the invention also includes kits containing a composition comprising a compound according to Formula I and another composition comprising one or more additional pharmaceutical agents used in the treatment of bipolar disorder such as Lithium, Zyprexa, and Depakote.

The invention also includes methods for treating Parkinson's disease, including treating memory and/or cognitive impairment associated with Parkinson's disease, comprising administering to a patient, simultaneously or sequentially, the compound of the invention and one or more additional agents used in the treatment of Parkinson's disease such as, but not limited to, Levodopa, Parlodel, Permax, Mirapex, Tasmar, Contan, Kemadin, Artane, and Cogentin. In methods using simultaneous administration, the agents can be present in a combined composition or can be administered separately. As a result, the invention also includes compositions comprising a compound according to Formula I and one or more additional pharmaceutical agents used in the treatment of Parkinson's disease, such as, but not limited to, Levodopa, Parlodel, Permax, Mirapex, Tasmar, Contan, Kemadin, Artane, and Cogentin. Similarly, the invention also includes kits containing a composition comprising a compound according to Formula I and another composition comprising one or more additional pharmaceutical agents gent used in the treatment of Parkinson's disease such as, but not limited to, Levodopa, Parlodel, Permax, Mirapex, Tasmar, Contan, Kemadin, Artane, and Cogentin.

In addition, the invention includes methods for treating memory and/or cognitive impairment associated with Alzheimer's disease comprising administering to a patient, simultaneously or sequentially, the compound of the invention and one or more additional agents used in the treatment of Alzheimer's disease such as, but not limited to, Reminyl, Cognex, Aricept, Exelon, Akatinol, Neotropin, Eldepryl, Estrogen and Cliquinol. In methods using simultaneous administration, the agents can be present in a combined composition or can be administered separately. As a result, the invention also includes compositions comprising a compound according to Formula I and one or more additional pharmaceutical agents used in the treatment of Alzheimer's disease such as, but not limited to, Reminyl, Cognex, Aricept, Exelon, Akatinol, Neotropin, Eldepryl, Estrogen and Cliquinol. Similarly, the invention also includes kits containing a composition comprising a compound according to Formula I and another composition comprising one or more additional pharmaceutical agents used in the treatment of Alzheimer's disease such as, but not limited to Reminyl, Cognex, Aricept, Exelon, Akatinol, Neotropin, Eldepryl, Estrogen and Cliquinol.

Another aspect of the invention includes methods for treating memory and/or cognitive impairment associated with dementia comprising administering to a patient, simultaneously or sequentially, the compound of the invention and one or more additional agents used in the treatment of dementia such as, but not limited to, Thioridazine, Haloperidol, Risperidone, Cognex, Aricept, and Exelon. In methods using simultaneous administration, the agents can be present in a combined composition or can be administered separately. As a result, the invention also includes compositions comprising a compound according to Formula I and one or more additional pharmaceutical agents used in the treatment of dementia such as, but not limited to, Thioridazine, Haloperidol, Risperidone, Cognex, Aricept, and Exelon. Similarly, the invention also includes kits containing a composition comprising a compound according to Formula I and another composition comprising one or more additional pharmaceutical agents used in the treatment of dementia such as, but not limited to, Thioridazine, Haloperidol, Risperidone, Cognex, Aricept, and Exelon.

A further aspect of the invention includes methods for treating memory and/or cognitive impairment associated with epilepsy comprising administering to a patient, simultaneously or sequentially, the compound of the invention and one or more additional agents used in the treatment of epilepsy such as, but not limited to, Dilantin, Luminol, Tegretol, Depakote, Depakene, Zarontin, Neurontin, Barbita, Solfeton, and Felbatol. In methods using simultaneous administration, the agents can be present in a combined composition or can be administered separately. As a result, the invention also includes compositions comprising a compound according to Formula I and one or more additional pharmaceutical agents used in the treatment of epilepsy such as, but not limited to, Dilantin, Luminol, Tegretol, Depakote, Depakene, Zarontin, Neurontin, Barbita, Solfeton, and Felbatol. Similarly, the invention also includes kits containing a composition comprising a compound according to Formula I and another composition comprising one or more additional pharmaceutical agents used in the treatment of epilepsy such as, but not limited to, Dilantin, Luminol, Tegretol, Depakote, Depakene, Zarontin, Neurontin, Barbita, Solfeton, and Felbatol.

A further aspect of the invention includes methods for treating memory and/or cognitive impairment associated with multiple sclerosis comprising administering to a patient, simultaneously or sequentially, the compound of the invention and one or more additional agents used in the treatment of multiple sclerosis such as, but not limited to, Detrol, Ditropan XL, OxyContin, Betaseron, Avonex, Azothioprine, Methotrexate, and Copaxone. In methods using simultaneous administration, the agents can be present in a combined composition or can be administered separately. As a result, the invention also includes compositions comprising a compound according to Formula I and one or more additional pharmaceutical agents used in the treatment of multiple sclerosis such as, but not limited to, Detrol, Ditropan XL, OxyContin, Betaseron, Avonex, Azothioprine, Methotrexate, and Copaxone. Similarly, the invention also includes kits containing a composition comprising a compound according to Formula I and another composition comprising one or more additional pharmaceutical agents used in the treatment of multiple sclerosis such as, but not limited to, Detrol, Ditropan XL, OxyContin, Betaseron, Avonex, Azothioprine, Methotrexate, and Copaxone.

The invention further includes methods for treating Huntington's disease, including treating memory and/or cognitive impairment associated with Huntington's disease, comprising administering to a patient, simultaneously or sequentially, the compound of the invention and one or more additional agents used in the treatment of Huntington's disease such as, but not limited to, Amitriptyline, Imipramine, Despiramine, Nortriptyline, Paroxetine, Fluoxetine, Setraline, Terabenazine, Haloperidol, Chloropromazine, Thioridazine, Sulpride, Quetiapine, Clozapine, and Risperidone. In methods using simultaneous administration, the agents can be present in a combined composition or can be administered separately. As a result, the invention also includes compositions comprising a compound according to Formula I and one or more additional pharmaceutical agents used in the treatment of Huntington's disease such as, but not limited to, Amitriptyline, Imipramine, Despiramine, Nortriptyline, Paroxetine, Fluoxetine, Setraline, Terabenazine, Haloperidol, Chloropromazine, Thioridazine, Sulpride, Quetiapine, Clozapine, and Risperidone. Similarly, the invention also includes kits containing a composition comprising a compound according to Formula I and another composition comprising one or more additional pharmaceutical agents used in the treatment of Huntington's disease such as, but not limited to, Amitriptyline, Imipramine, Despiramine, Nortriptyline, Paroxetine, Fluoxetine, Setraline, Terabenazine, Haloperidol, Chloropromazine, Thioridazine, Sulpride, Quetiapine, Clozapine, and Risperidone.

A further aspect of the invention includes methods for treating diabetes, including treating cognitive impairment associate with diabetes, comprising administering to a patient, simultaneously or sequentially, the compound of the invention and one or more additional agents used in the treatment of diabetes such as, but not limited to, PPAR ligands (e.g., rosiglitazone, troglitazone and pioglitazone), insulin secretagogues (e.g., sulfonylurea drugs such as glyburide, glimepiride, chlorpropamide, tolbutamide, and glipizide and non-sulfonyl secretagogues), α-glucosidase inhibitors (e.g., acarbose, miglitol, and voglibose), insulin sensitizers (e.g., PPAR-γ agonists, glitazones; biguanides, PTP-1B inhibitors, DPP-IV inhibitors and 11beta-HSD inhibitors), hepatic glucose output lowering compounds (e.g., glucagon antagonists, metaformin, Glucophage and Glucophage XR), insulin and insulin derivatives (both long and short acting forms and formulations of insulin), anti-obesity drugs (e.g., β-3 agonists, CB-1 antagonists/inverse agonists, neuropeptide Y5 inhibitors, Ciliary Neurotrophic Factor and derivatives such as Axokine), appetite suppressants (e.g., sibutramine), and lipase inhibitors (i.t., orlistat). Similarly, the invention also includes kits containing a composition comprising a compound according to Formula I and another composition comprising one or more additional pharmaceutical agents used in the treatment of diabetes such as, but not limited to, Rosiglitazone, Troglitazone Pioglitazone, Glyburide, Glimepiride, Chlorpropamide, Tolbutamide, Glipizide, non-sulfonyl secretagogues, Acarbose, Miglitol, Voglibose, PPAR-γ agonists, glitazones; biguanides, PTP-1B inhibitors, DPP-IV inhibitors, 11beta-HSD inhibitors, glucagon antagonists, metaformin, Glucophage, Glucophage XR, insulin and insulin derivatives, β-3 agonists, CB-1 antagonists/inverse agonists, neuropeptide YS inhibitors, Ciliary, Axokine, and Orlistat.

In methods using simultaneous administration, the agents can be present in a combined composition or can be administered separately. Similarly, the invention also includes kits containing a composition comprising a compound according to Formula I and another composition useful for treating obesity.

The dosages of the compounds of the present invention depend upon a variety of factors including the particular syndrome to be treated, the severity of the symptoms, the route of administration, the frequency of the dosage interval, the particular compound utilized, the efficacy, toxicology profile, pharmacokinetic profile of the compound, and the presence of any deleterious side-effects, among other considerations. One of ordinary skill in the art of treating such diseases will be able, without undue experimentation and in reliance upon personal knowledge and the disclosure of this Application, to ascertain a therapeutically effective amount of the compounds of the present invention for a given disease.

The compounds of the invention are typically administered at dosage levels and in a mammal customary for 5-HT₆ ligands, such as those known compounds mentioned above. For example, the compounds can be administered, in single or multiple doses, by oral administration at a dosage level of generally 0.001-100 mg/kg/day, for example, 0.01-100 mg/kg/day, or 0.1-70 mg/kg/day, or 0.5-10 mg/kg/day. Unit dosage forms can contain generally 0.01-1000 mg of active compound, for example, 0.1-50 mg of active compound. For intravenous administration, the compounds can be administered, in single or multiple dosages, at a dosage level of, for example, 0.001-50 mg/kg/day, or 0.001-10 mg/kg/day, or 0.01-1 mg/kg/day. Unit dosage forms can contain, for example, 0.1-10 mg of active compound.

In carrying out the procedures of the present invention, it is of course to be understood that reference to particular buffers, media, reagents, cells, culture conditions and the like are not intended to be limiting, but are to be read so as to include all related materials that one of ordinary skill in the art would recognize as being of interest or value in the particular context in which that discussion is presented. For example, it is often possible to substitute one buffer system or culture medium for another and still achieve similar, if not identical, results. Those of skill in the art will have sufficient knowledge of such systems and methodologies so as to be able, without undue experimentation, to make such substitutions as will optimally serve their purposes in using the methods and procedures disclosed herein.

The present invention will now be further described by way of the following non-limiting examples. In applying the disclosure of these examples, it should be kept clearly in mind that other and different embodiments of the methods disclosed according to the present invention will no doubt suggest themselves to those of skill in the relevant art.

In the foregoing and in the following examples, all temperatures are set forth uncorrected in ° Celsius; and, unless otherwise indicated, all parts and percentages are by weight.

The entire disclosures of all applications, patents and publications, cited above and below, are hereby incorporated by reference in their entirety.

### ABBREVIATIONS AND ACRONYMS

When the following abbreviations are used throughout this disclosure, they have the following meaning:
Ac acetyl
aq aqueous
BINAP 2,2′-bis(diphenylphosphino)-1,1′-binaphthyl
Bn benzyl
Boc tert-butyloxycarbonyl
(Boc)₂O di-tert-butyldicarbonate
n-BuLi, n-butyllithium
Cbz benzyloxycarbonyl
ClCOOEt ethyl chloroformate
conc concentrated
d doublet
dd doublet of doublet
ddd doublet of doublet of doublet
DEAD diethylazodiacetate
DMF N,N-dimethyl formamide
DMSO dimethylsulfoxide
DMSO-d₆ dimethylsulfoxide-d₆
E entgegen
eq equivalent
ES electrospray (mass spectrometry)
Et ethyl
Etl iodoethane
Et₂O diethyl ether
Et₃N triethylamine
EtOAc ethyl acetate
EtOH ethanol
g gram(s)
h hour(s)
[³H] MLA tritiated methyllycaconitine citrate
¹H NMR proton nuclear magnetic resonance
HPLC high-performance liquid chromatography
HPLC ES-MS high-performance liquid chromatography-electrospray mass spectroscopy
HOAc acetic acid
L liter
LC-MS liquid chromatography/mass spectroscopy
m multiplet
M molar
mg milligram(s)
mL milliliter
m/z mass-to-charge ratio
Me methyl
MeCN acetonitrile
MeI iodomethane
MeOH methanol
MeOD methanol-d₄, CD₃OD
MHz megahertz
min minute(s)
mmol millimole(s)
mol mole
MS mass spectrometry
N normal
NaHMDS sodium bis(trimethylsilyl)amide
NBS N-bromosuccinimide
NCS N-chlorosuccinimide
Pd(OAc)₂ palladium acetate
Pd/C palladium on carbon
PE petroleum ether
Ph phenyl
ppm parts per million
Pr propyl
q quartet
rt room temperature
TEBA triethylbenzylammonium chloride
THF tetrahydrofuran
t_{R} retention time (HPLC)
singlet
t triplet
TFA trifluoroacetic acid
TLC thin layer chromatography
TMS tetramethylsilane
w/w weight per unit weight

### Experimental Examples

All spectra were recorded at 300 MHz on a Bruker Instruments NMR unless otherwise stated. Coupling constants (a) are in Hertz (Hz) and peaks are listed relative to TMS (δ 0.00 ppm).

Analytical HPLC was performed on a 4.6 mm×100 mm Waters Sunfire RP C18 5 mm column using a gradient of typically (i) 5/95 to 60/40 acetonitrile (0.1% formic acid)/water (0.1% formic acid) over 8 min (Analytical Method A), (ii) 10/90 to 80/20 acetonitrile (0.1% formic acid)/water (0.1% formic acid) over 8 min (Analytical Method B), or (iii) 20/80 to 80/20 acetonitrile (0.1% formic acid)/water (0.1% formic acid) over 8 min (Analytical Method C).

Preparative HPLC was performed at a flow rate of 45 mL/min on a 30 mm×100 mm C18 Sunfire Prep 5μ or a 30 mm×100 mm C18 Atlantis Prep 5μ column using one of the following gradients: (i) 20/80 to 80/20 acetonitrile (0.1% formic acid)/water (0.1% formic acid) over 10 min (Preparative Method A), (ii) 10/90 to 80/20 acetonitrile (0.1% formic acid)/water (0.1% formic acid) over 10 min (Preparative Method B), (iii) 15/85 to 60/40 acetonitrile (0.1% formic acid)/water (0.1% formic acid) over 10 min (Preparative Method C), (iv) 5/95 to 80/20 acetonitrile (0.1% formic acid)/water (0.1% formic acid) over 8 min (Preparative Method D), or (v) 5/95 to 50/50 acetonitrile (0.1% formic acid)/water (0.1% formic acid) over 8 min (Preparative Method E).

### I. Sulfonyl Chloride Preparations

Sulfonyl chlorides used herein are either commercially available from suppliers such as Sigma-Aldrich, Milwaukee, Wis. US; Lancaster Synthesis, Windham, N.H. USA; or Maybridge Chemical Co. Ltd., Cornwall, UK; or prepared by means known in the art or according to the procedures outlined below.

For example, benzenesulfonyl chloride, 2-chlorobenzenesulfonyl chloride, 3-chlorobenzenesulfonyl chloride, 4-chlorobenzenesulfonyl chloride, 2-fluorobenzenesulfonyl chloride, 3-fluorobenzenesulfonyl chloride, 4-fluorobenzenesulfonyl chloride, 2-methoxybenzenesulfonyl chloride, 3-methoxybenzenesulfonyl chloride, 4-methoxybenzenesulfonyl chloride, 2-difluoromethoxybenzenesulfonyl chloride, 3-difluoromethoxybenzenesulfonyl chloride, 4-difluoromethoxybenzenesulfonyl chloride, 2-trifluoromethoxybenzenesulfonyl chloride, 3-trifluoromethoxybenzenesulfonyl chloride, 4-trifluoromethoxybenzenesulfonyl chloride, 3-trifluoromethylbenzenesulfonyl chloride, 2-methylbenzenesulfonyl chloride, 3-methylbenzenesulfonyl chloride, 4-methylbenzenesulfonyl chloride, 2-cyanobenzenesulfonyl chloride, 3-cyanobenzenesulfonyl chloride, 4-cyanobenzenesulfonyl chloride, 3-acetylbenzenesulfonyl chloride, 3,4-dimethoxybenzenesulfonyl chloride, 2,4-dimethoxybenzenesulfonyl chloride, 2,5-dimethoxybenzenesulfonyl chloride, 3-cyano-4-fluorobenzenesulfonyl chloride, 4-(2-oxo-pyrrolidin-1-yl)-benzenesulfonyl chloride, 3-(pyridine-2-carbonyl)benzenesulfonyl chloride, 2-cyano-5-methylbenzenesulfonyl chloride, 2-chloro-4-cyanobenzenesulfonyl chloride, 3-methyl-6-methoxybenzenesulfonyl chloride, 2,4-difluorobenzenesulfonyl chloride, 2,5-difluorobenzenesulfonyl chloride, 4-fluoro-3-methylbenzenesulfonyl chloride, 2-fluoro-5-methylbenzenesulfonyl chloride, pyridine-3-sulfonyl chloride, 6-phenoxy-3-pyridinesulfonyl chloride, 6-(morpholin-4-yl)-pyridine-3-sulfonyl chloride, 5-trifluoromethyl-2-pyridinesulfonyl chloride, 1-naphthalenesulfonyl chloride, 5-bromo-2,3-dihydrobenzo[b]furan-7-sulfonyl chloride, 4-methyl-3,4-dihydro-2H-pyrido[3,2-b][1,4]oxazine-7-sulfonyl chloride, 1-methyl-1H-indole-5-sulfonyl chloride, 2-methyl-1,3-benzothiazole-6-sulfonyl chloride, 1-acetyl-2,3-dihydro-1H-indole-5-sulfonyl chloride, 2,3-dihydro-1,4-benzodioxine-6-sulfonyl chloride, 3,4-dihydro-2H-1,5-benzodioxepine-7-sulfonyl chloride, 5-chloro-3-methylbenzo[b]thiophene-2-sulfonyl chloride, quinoline-3-sulfonyl chloride, 2,3-dihydro-1-benzofuran-5-sulfonyl chloride, 2-oxo-1,2,3,4-tetrahydroquinoline-6-sulfonyl chloride, 4-methyl-3,4-dihydro-2H-1,4-benzoxazine-7-sulfonyl chloride, and 6-chloroimidazo[2,1-b][1,3]thiazole-5-sulfonyl chloride were purchased from a commercial supplier, such as those listed above, and were used directly without additional purification steps.

### Intermediate 1: Synthesis of 3-(dimethylamino)benzene-1-sulfonyl chloride

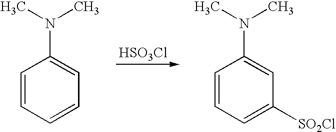

Sulfurochloridic acid (100 g) was cooled to 0° C. and N,N-dimethylaminobenzene (165 mmol) was added dropwise with stirring, maintaining a temperature of 0° C. The resulting solution was then heated to 120° C. and stirred for 3 h. After cooling to rt; dichloromethane (40 mL) was added and the resulting mixture was added dropwise to 100 mL of cold (0° C.) brine water. The resulting solution was extracted with dichloromethane (3×500 mL) and the combined organic layers were, dried (sodium sulfate) and filtered. The filtrate was concentrated and the residue was purified by Flash chromatography (1/100 ethyl acetate/petroleum ether). The collected fractions were combined and concentrated to give 4.1 g (110%) of 3-(dimethylamino)benzene-1-sulfonyl chloride in 11% yield as a yellow solid. ¹H NMR (CDCl₃) δ 7.41 (t, 1H), 7.31 (d, 1H), 7.23 (s, 1H), 6.98 (m, 1H), 3.05 (s, 6H).

### Intermediate 2: Synthesis of 4-(N-methylacetamido)benzene-1-sulfonyl chloride

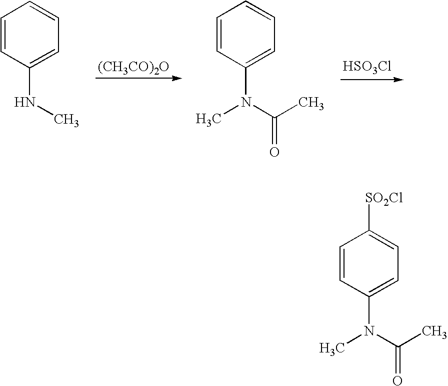

### 1. Synthesis of N-methyl-N-phenylacetamide

Acetic anhydride (481 mmol) was added to N-methylbenzenamine (100 mmol) and the resulting solution was maintained at rt for 15 h. The reaction mixture was diluted with iced water (200 mL) and was extracted with dichloromethane (2×100 mL). The combined organic layers were dried (sodium sulfate) and concentrated to afford N-methyl-N-phenylacetamide in 70% yield as a white solid.

### 2. Synthesis of 4-(N-methylacetamido)benzene-1-sulfonyl chloride

A solution of N-methyl-N-phenylacetamide (73.8 mmol) in dichloromethane (20 mL) was added dropwise to sulfurochloridic acid (690 mmol) at 5° C. and the resulting solution was allowed to warm to rt and was maintained 16 h. The reaction mixture was diluted with iced water (100 mL) and was extracted with dichloromethane (2×50 mL). The combined organic layers were dried (sodium sulfate) and concentrated. The residue was purified by Flash chromatography (10/1 ethyl acetate/petroleum ether) to give 4-(N-methylacetamido)benzene-1-sulfonyl chloride in 11% yield as a white solid. ¹H NMR (CDCl₃) δ 8.09 (d, 2H), 7.48 (d, 2H), 3.38 (s, 3H), 2.17 (s, 3H).

### Intermediate 3: Synthesis of 4-morpholinobenzene-1-sulfonyl chloride

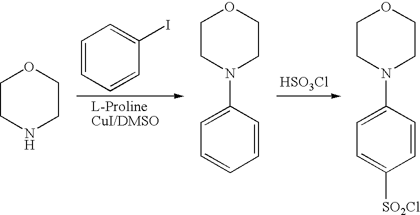

### 1. Synthesis of 4-phenylmorpholine

A mixture of L-proline (27.1 mmol) and copper(I) iodide (13.7 mmol) was diluted with 1-iodobenzene (138 mmol), morpholine (138 mmol), and dimethylsulfoxide (120 mL) and the reaction mixture was heated at 90° C. for 4 h. The reaction mixture was diluted with ice water (300 mL) and was extracted with dichloromethane (2×200 mL). The combined organic layers were dried (sodium sulfate) and concentrated. The residue was purified by Flash chromatography (petroleum ether) to give 4-phenylmorpholine in 42% yield as a white solid.

### 2. Synthesis of 4-morpholinobenzene-1-sulfonyl chloride

Sulfurochloridic acid (613 mmol) was cooled to 0° C. and 4-phenylmorpholine (123 mmol) was added in several batches, while keeping the temperature at 0° C. The resulting solution was then stirred at 90° C. for 20 h. The reaction mixture was then added dropwise to 200 mL of cold (0° C.) brine. The resulting solution was extracted with ethyl acetate (2×200 mL) and the combined organic layers were dried (magnesium sulfate) and filtered. The filtrate was concentrated, and the residue was purified by Flash chromatography (20/1 ethyl acetate/petroleum ether) to give 4-morpholinobenzene-1-sulfonyl chloride in 15% yield as a yellow solid. ¹H NMR (CDCl₃) δ 7.9 (d, 2H), 6.9 (d, 1H), 7.5 (d, 2H), 3.87 (t, 2H), 3.4 (t, 2H).

### Intermediate 4: Synthesis of 4-(2-oxopyrrolidin-1-yl)benzene-1-sulfonyl chloride

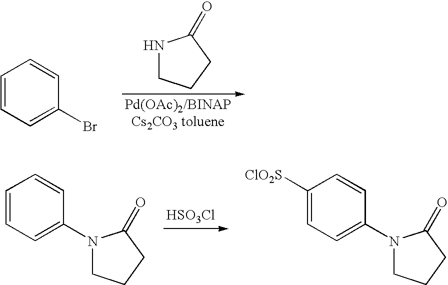

### 1. Synthesis of 1-phenylpyrrolidin-2-one

Pyrrolidin-2-one (25.7 mmol), palladium(II) acetate (0.250 mmol), BINAP (0-390 mmol), and cesium carbonate (38.3 mmol) were added to a solution of 1-bromobenzene (25.5 mmol) in toluene (50 mL) and the reaction mixture was heated at reflux for 16 h. The reaction mixture was concentrated and the residue was purified by Flash chromatography (1/10 ethyl acetate/petroleum ether) to provide 1-phenylpyrrolidin-2-one in 24% yield as yellow oil.

### 2. Synthesis of 4-(2-oxopyrrolidin-1-yl)benzene-1-sulfonyl chloride

1-Phenylpyrrolidin-2-one (6.21 mmol) was added to sulfurochloridic acid (10 mL) and the reaction mixture was maintained at rt for 16 h. The reaction mixture was diluted with ice water (100 mL) and the resulting mixture was extracted with dichloromethane (100 mL). The organic layer was dried (magnesium sulfate) and concentrated to provide 4-(2-oxopyrrolidin-1-yl)benzene-1-sulfonyl chloride in 43% yield as a yellow solid. Data: ¹HNMR (400 MHz, CDCl₃) δ 2.22 (m, 2H), 2.71 (t, 2H), 3.95 (t, 2H), 7.88 (t, 2H), 8.05 (t, 2H).

### Intermediate 5: Synthesis of 3-(2-oxopyrrolidin-1-yl)benzene-1-sulfonyl chloride

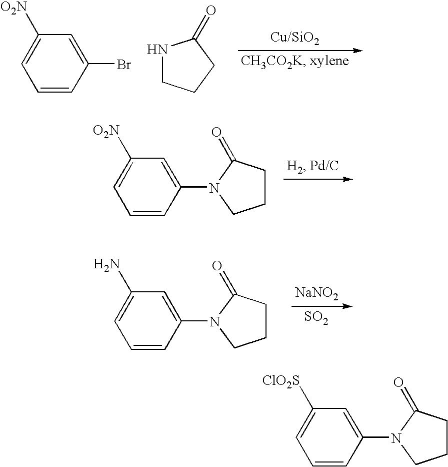

### 1. Synthesis of 1-(3-nitrophenyl)pyrrolidin-2-one

A suspension of 1-bromo-3-nitrobenzene (30.0 mmol), pyrrolidin-2-one (45.1 mmol), potassium acetate (60.0 mmol), and copper impregnated silica gel (60.0 mmol) in xylene (50 mL) was heated at 130° C. for 16 h. The insoluble solids were removed by filtration and the filter cake was washed with ethyl acetate (4×300 mL). The combined organic layers were concentrated and the residue was purified by Flash chromatography (10/1 to 5/1 petroleum ether/ethyl acetate) to provide 1-(3-nitrophenyl)pyrrolidin-2-one in 52% yield as a light yellow solid.

### 2. Synthesis of 1-(3-aminophenyl)pyrrolidin-2-one

A suspension of 1-(3-nitrophenyl)pyrrolidin-2-one (27.7 mmol) and 10% palladium oil carbon (5 g) was maintained under an atmosphere of hydrogen gas at 35° C. for 16 h. The insoluble solids were removed by filtration and the filter cake was washed with ethyl acetate (3×300 mL). The combined organic layers were concentrated to provide 1-(3-aminophenyl)pyrrolidin-2-one in 92% yield as a white solid.

### 3. Synthesis of 3-(2-oxopyrrolidin-1-yl)benzene-1-sulfonyl chloride

Hydrochloric acid (11 mL) was added to a solution of 1-(3-aminophenyl)pyrrolidin-2-one (35.2 mmol) in acetic acid (21 mL) and acetonitrile (250 mL) at 0° C. A solution of sodium nitrite (42.0 mmol) in water (3 mL) was subsequently added and the mixture was maintained for 60 min at 0° C. Sulfur dioxide gas was bubbled through the solution for 2 h while the temperature was maintained at 0° C. A solution of copper(II) chloride dihydrate (38.8 mmol) in water (5 mL) was added dropwise and sulfur dioxide gas was bubbled through the solution for an additional 60 min. The reaction mixture was allowed to warm to rt and was maintained for 16 h. The precipitated solids were collected by filtration, washed with ice water (3×10 mL), and dried to provide 3-(2-oxopyrrolidin-1-yl)benzene-1-sulfonyl chloride in 22% yield as a brown solid. Data: ¹HNMR (400 MHz, CDCl₃) δ 8.27 (d, 1H), 8.14 (s, 1H), 7.80 (d, 1H), 7.61 (t, 1H), 3.94 (t, 2Ht), 2.68 (t, 2H), 2.24 (t, 2H). LC/MS (ES) m/z 329 [M+BnNH−HB]⁻.

### Intermediate 6: Synthesis of 4-(pyrrolidin-1-yl)benzene-1-sulfonyl chloride

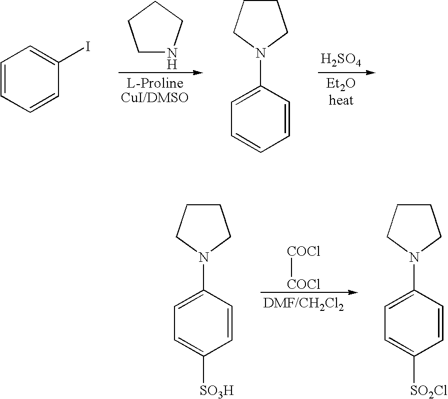

### 1. Synthesis of 1-phenylpyrrolidine

Pyrrolidine (304 mmol), L-proline (9.74 mmol), and copper(I) iodide (5.05 mmol) were added sequentially to a solution of 1-iodobenzene (49.0 mmol) in dimethylsulfoxide (40 mL) and the reaction mixture was heated at 60° C. for 20 h. The reaction mixture was diluted with iced water (400 mL) and was extracted with ethyl acetate (3×150 mL). The combined organic layers were dried (sodium sulfate), filtered and concentrated. The residue was purified by Flash chromatography (1/100 ethyl acetate/petroleum ether) to afford 1-phenylpyrrolidine in 57% yield as brown oil.

### 2. Synthesis of 4-(pyrrolidin-1-yl)benzenesulfonic acid

A solution of sulfuric acid (68.0 mmol) in diethylether (80 mL) was added to a solution of 1-phenylpyrrolidine (68.0 mmol) in diethylether (20 mL) at 0° C. The diethylether was decanted and the resulting solution was maintained for 3 h at 170° C. and concentrated to afford 4-(pyrrolidin-1-yl)benzenesulfonic acid in 43% yield as a white solid.

### 3. Synthesis of 4-(pyrrolidin-1-yl)benzene-1-sulfonyl chloride

Oxalyl chloride (78.7 mmol) was added dropwise to a solution of 4-(pyrrolidin-1-yl)benzenesulfonic acid (32.2 mmol) and N,N-dimethylformamide (0.5 mL) in dichloromethane (40 mL) and the resulting solution was maintained at rt for 1 h. The reaction mixture was diluted with ice water (40 mL) and the layers were separated. The aqueous layer was extracted with dichloromethane (3×20 mL) and the combined organic layers were dried (sodium sulfate), filtered and concentrated. The residue was purified by Flash chromatography (1/100 ethyl acetate/petroleum ether) to afford 4-(pyrrolidin-1-yl)benzene-1-sulfonyl chloride in 19% yield as a yellow solid. ¹H NMR (CDCl₃) δ 7.78 (d, 2H), 6.55 (d, 2H), 3.41 (t, 4H), 2.03 (t, 4H).

### 4. Synthesis of 4-(3-methoxypyrrolidin-1-yl)benzene-1-sulfonyl chloride, 4-[(3R)-3-methoxypyrrolidin-1-yl]benzene-1-sulfonyl chloride and 4-[(3S)-3-methoxypyrrolidin-1-yl]benzene-1-sulfonyl chloride

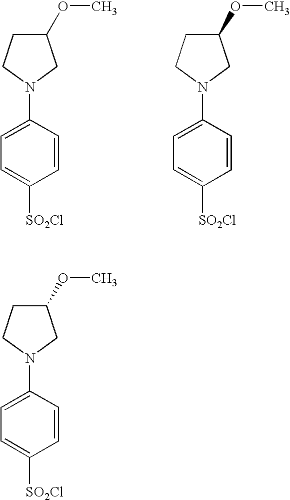

4-(3-Methoxypyrrolidin-1-yl)benzene-1-sulfonyl chloride, 4-[(3R)-3-methoxypyrrolidin-1-yl]benzene-1-sulfonyl chloride, and 4-[(3S)-3-methoxypyrrolidin-1-yl]benzene-1-sulfonyl chloride were prepared from 3-methoxypyrrolidine, (R)-3-methoxypyrrolidine and (S)-3-methoxypyrrolidine, respectively, using the procedure for the preparation of Intermediate 6.

### Intermediate 7: Synthesis of 3-(pyrrolidin-1-yl)benzene-1-sulfonyl chloride

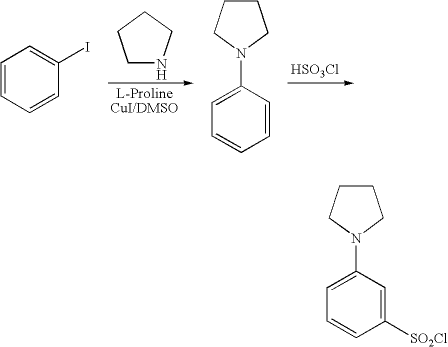

1-Phenylpyrrolidine (29.3 mmol) was added dropwise to sulfurochloridic acid (20 mL) at 0° C. and the reaction mixture was heated at 60° C. 16 h. The reaction mixture was diluted with cold (0° C.) brine (200 mL) and was extracted with ethyl acetate (3×100 mL), and the combined organic layers were dried (sodium sulfate), filtered and concentrated. The residue was purified by Flash chromatography (1/500 ethyl acetate/petroleum ether) to give 3-(pyrrolidin-1-yl)benzene-1-sulfonyl chloride in 7% yield as a yellow solid. ¹H NMR (CDCl₃) δ 7.36 (m, 1H), 7.24 (d, 1H), 7.07 (s, 1H), 6.82 (d, 1H), 3.34 (t, 4H), 2.05 (t, 4H).

### Intermediate 8: Synthesis of 3-(3-methoxypyrrolidin-1-yl)benzene-1-sulfonyl chloride

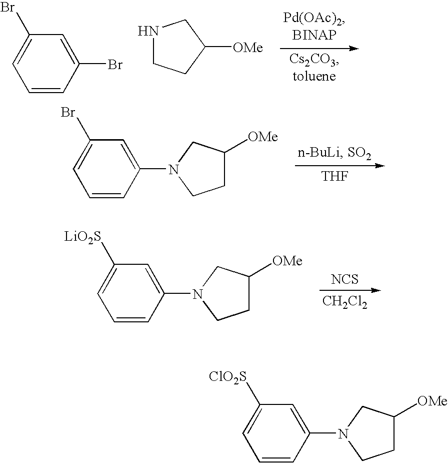

### 1. Synthesis of 1-(3-bromophenyl)-3-methoxypyrrolidine

3-Methoxypyrrolidine (60.4 mmol), palladium(II) acetate (0.500 mmol), BINAP (1.51 mmol), and cesium carbonate (126 mmol) were added to a solution of 1,3-dibromobenzene (50.4 mmol) in toluene (100 mL) under an atmosphere of nitrogen and the reaction mixture was heated at reflux for 16 h. The insoluble solids were removed by filtration and the filtrate was concentrated. The residue was purified by Flash chromatography (1/30 ethyl acetate/petroleum ether) to provide 1-(3-bromophenyl)-3-methoxypyrrolidine in 64% yield as yellow oil.

### 2. Synthesis of lithium 3-(3-methoxypyrrolidin-1-yl)benzenesulfinate

n-Butyllithium (39 mmol) was added to a solution of 1-(3-bromophenyl)-3-methoxypyrrolidine (32.4 mmol) in tetrahydrofuran (100 mL) at −78° C. and the reaction mixture was maintained for 60 min. Sulfur dioxide (4 mL) was added and the reaction mixture was maintained at −78° C. for an additional 2 h. The reaction mixture was concentrated and the residue was diluted with hexane. The precipitated solids were collected by filtration, washed with hexane (2×50 mL), and dried to provide lithium 3-(3-methoxypyrrolidin-1-yl)benzenesulfinate in 90% yield as a yellow solid.

### 3. Synthesis of 3-(3-methoxypyrrolidin-1-yl)benzene-1-sulfonyl chloride

N-Chlorosuccinamide (33.6 mmol) was added in over 10 min to a solution of lithium 3-(3-methoxypyrrolidin-1-yl)benzenesulfinate (29.2 mmol) in dichloromethane (100 mL) at 0° C. and the reaction mixture was maintained for an additional 15 min. The reaction mixture was then allowed to warm to rt and was maintained for 25 min. The resulting mixture was washed with sodium hydrogen sulfate (2×50 mL) and brine (2×50 mL), dried (sodium sulfate), and concentrated. The residue was purified by Flash chromatography (2/3 ethyl acetate/petroleum ether) to provide 3-(3-methoxypyrrolidin-1-yl)benzene-1-sulfonyl chloride in 83% yield as a yellow oil. Data: ¹HNMR (400 Hz, CDCl₃) δ 2.24 (m, 1H), 2.30 (m, 1H); 3.54-3.45 (m, 2H) 3.61-3.56 (m, 2H), 4.20 (s, 3H), 6.90 (d, J=8, 1H), 7.34 (d, J=8, 1H), 7.37 (dd, J=8, 1H), 7.49 (dd, J=8, 8, 1H). LC/MS (ES) m/z 347 [M+BnNH+H]⁺.

### Intermediate 9: Synthesis of 3-[(3R)-3-methoxypyrrolidin-1-yl]benzene-1-sulfonyl chloride and 3-[(3S)-3-methoxypyrrolidin-1-yl]benzene-1-sulfonyl chloride

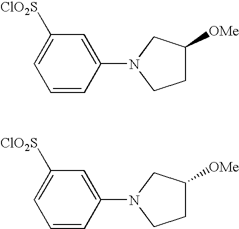

3-[(3R)-3-Methoxypyrrolidin-1-yl]benzene-1-sulfonyl chloride and 3-[(3S)-3-methoxypyrrolidin-1-yl]benzene-1-sulfonyl chloride were prepared from (R)-3-methoxypyrrolidine and (S)-3-methoxypyrrolidine, respectively, using the procedure for the preparation of Intermediate 8.

### Intermediate 10: Synthesis of 2-(3-Methoxypyrrolidin-1-yl)benzene-1-sulfonyl chloride

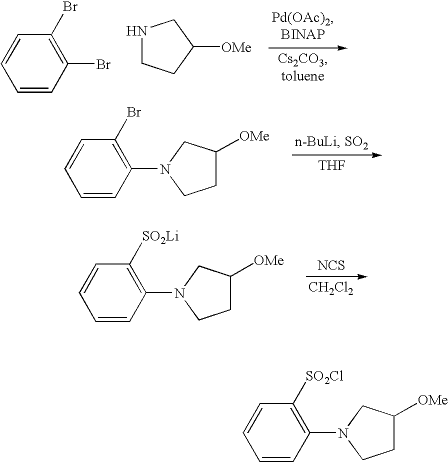

2-(3-Methoxypyrrolidin-1-yl)benzene-1-sulfonyl chloride was prepared from 3-methoxypyrrolidine and 1,2-dibromobenzene using the procedure for the preparation of Intermediate 8. Data: ¹H NMR (CDCl₃) δ 8.0 (m, 1H), 7.4 (m, 1H), 7.0 (m, 1H), 6.9 (m, 1H), 4.0 (m, 1H), 3.6 (m, 4H), 3.3 (s, 3H), 2.1 (m, 2H). LC/MS (ES) m/z 340 [M+C5H11N2-Cl+H]⁺.

### Intermediate 1: Synthesis of 5-[(3S)-3-methoxypyrrolidin-1-yl]pyridine-3-sulfonyl chloride

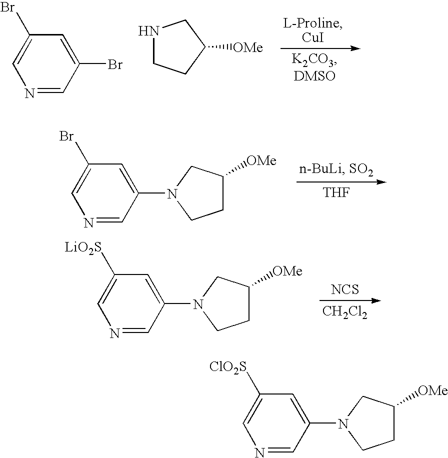

5-[(3S)-3-Methoxypyrrolidin-1-yl]pyridine-3-sulfonyl chloride was prepared from 3,5-dibromopyridine using the procedure for the preparation of intermediate 8. The coupling method used, copper(I) iodide and L-proline, was the same as that used for intermediate 7. Data: ¹H NMR (CDCl₃) δ 8.48 (s, 1H), 8.23 (s, 1H), 7.30 (s, 1H), 4.17 (s, 1H), 3.45-3.56 (m, 4H), 3.39 (s, 3H), 2.29 (m, 1H), 2.15 (m, 1H). LC/MS (ES) m/z 348 [M+H+ BnNH]⁺.

### Intermediate 12: Synthesis of 5-methoxypyridine-3-sulfonyl chloride

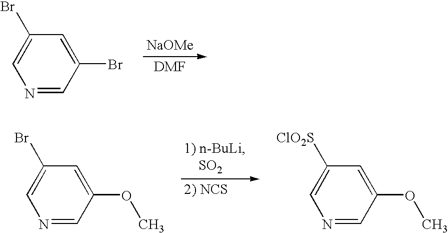

### 1. Synthesis of

Sodium methoxide (255 mmol) was added to a solution of 3,5-dibromopyridine (124 mmol) in N,N-dimethylformamide (200 mL) and the reaction mixture was heated at 40° C. for 24 h. The resulting mixture was diluted with water (200 mL) and was extracted with ethyl acetate (3×100 mL). The combined organic layers were dried (magnesium sulfate) and concentrated. The residue was purified by Flash chromatography (40/1 petroleum ether/ethyl acetate) to provide 3-bromo-5-methoxypyridine in 59% yield as a white solid.

### 2. Synthesis of 5-methoxypyridine-3-sulfonyl chloride

n-Butyllithium (12.8 mmol) was added to a solution of 3-bromo-5-methoxypyridine (26.6 mmol) in tetrahydrofuran (80 mL) at −78° C. and the reaction mixture was maintained for 30 min. Sulfur dioxide (29.2 mmol) was added and the reaction mixture was allowed to warm to rt and was maintained for an additional 16 h. The reaction mixture was diluted with hexane (80 mL) and the precipitated solids were collected by filtration to provide the lithium salt. The salt was suspended in dichloromethane (30 mL), cooled to 0° C. and N-chlorosuccinamide (39.7 mmol) was added in portions over 10 min. The reaction mixture was allowed to warm to rt and was maintained for 60 min. The resulting mixture was diluted with dichloromethane (30 mL) and was washed with 2 M sodium hydrogen sulfite (2×50 mL) and brine (3×50 mL), dried (sodium sulfate), and concentrated. The residue was purified by Flash chromatography (1/5 ethyl acetate/petroleum ether) to provide 5-methoxypyridine-3-sulfonyl chloride in 27% yield as a yellow oil. Data: ¹H NMR (400 MHz, CDCl₃) δ 8.84 (s, 1H), 8.63 (s, 1H), 7.70 (s, 1H), 3.98 (s, 3H). LC/MS (ES) m/z 277 [M+BnNH−H]⁻. Ref: Michael L. Curtin, Steven K. Davidsen, et al. J. Med. Chem. 1998, 41, 74-95.

### Intermediate 13: Synthesis of 2-[(38)-3-methoxypyrrolidin-1-yl]pyridine-5-sulfonyl chloride

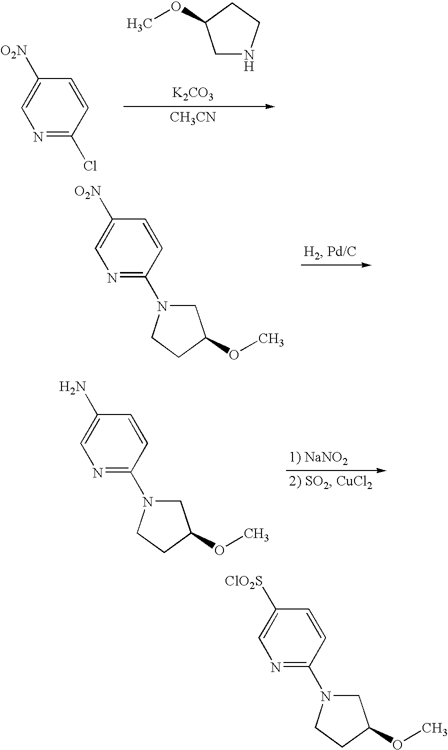

### 1. Synthesis of 2-[(3S)-3-methoxypyrrolidin-1-yl]-5-nitropyridine

A suspension of 2-chloro-5-nitropyridine (29.9 mmol), (S)-3-methoxypyrrolidine hydrochloride (45.0 mmol), and potassium carbonate (60.0 mmol) in acetonitrile (250 mL) was heated at reflux for 4 h. The reaction mixture was concentrated to provide 2-[(3S)-3-methoxypyrrolidin-1-yl]-5-nitropyridine as a yellow solid.

### 2. Synthesis of 2-[(3S)-3-methoxypyrrolidin-1-yl]pyridine-5-amine

A suspension of 2-[(3S)-3-methoxypyrrolidin-1-yl]-5-nitropyridine (32.7 mmol) and 10% palladium on carbon in methanol (200 mL) was maintained under an atmosphere of hydrogen gas for 7 h at rt. The insoluble solids were removed by filtration and the filtrate was concentrated to provide 2-[(3S)-3-methoxypyrrolidin-1-yl]pyridine-5-amine as purple oil.

### 3. Synthesis of 2-[(3S)-3-methoxypyrrolidin-1-yl]pyridine-5-sulfonyl chloride

Hydrochloric acid (8 mL) was added to a solution of 2-[(3S)-3-methoxypyrrolidin-1-yl]pyridine-5-amine (31.6 mmol) in acetic acid (15 mL) at 0° C. A solution of sodium nitrite (3.1.9 mmol) in water (5 mL) was subsequently added and the mixture was maintained for 30 min at 0° C. The solution of the diazo salt was added over 5 min to acetic acid (35 mL) saturated with sulfur dioxide gas. A solution of copper(II) chloride dihydrate (31.6 mmol) in water (2 mL) was added and the reaction mixture was allowed to warm to rt and was maintained for 2 h. The reaction mixture was diluted with ice water (100 mL) and the resulting mixture was extracted with ether (3×200 mL). The combined organic layers were washed with brine, dried (sodium sulfate), and concentrated to provide 2-[(3S)-3-methoxypyrrolidin-1-yl]pyridine-5-sulfonyl chloride as light black oil. Data: LC/MS (ES) m/z 348 [M+PhCH2NH2+H]⁺.

### Intermediate 14: Synthesis of 3-(3-(tetrahydro-2H-pyran-2-yloxy)pyrrolidin-1-yl)benzene-1-sulfonyl chloride

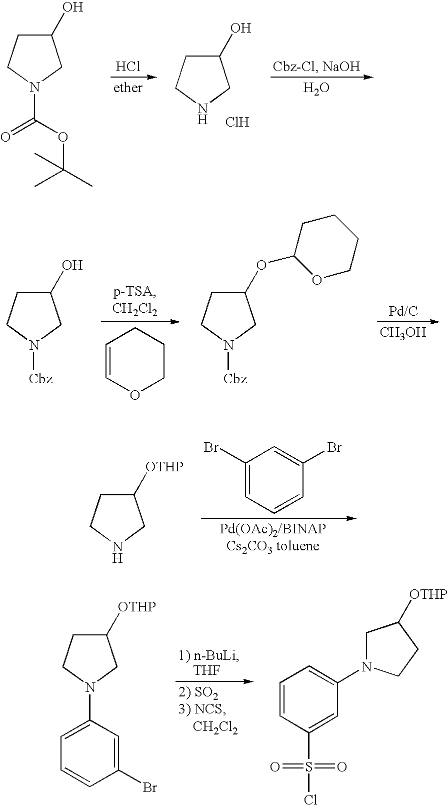

### 1. Synthesis of pyrrolidin-3-ol hydrochloride

Gaseous hydrochloric acid was bubbled through a solution of tert-butyl 3-hydroxypyrrolidine-1-carboxylate (219 mmol) in ethyl ether (300 mL) at rt over a time period of 3 h and the reaction mixture was maintained for an additional 16 h at rt. The reaction mixture was concentrated to provide crude pyrrolidin-3-ol hydrochloride as a white solid.

### 2. Synthesis of benzyl 3-hydroxypyrrolidine-1-carboxylate

Pyrrolidin-3-ol hydrochloride (163 mmol) was dissolved in water (60 mL), cooled to 5° C., and the pH of the reaction mixture was adjusted to 7 with 10% sodium hydroxide. Benzyl chloroformate (216 mmol) was added dropwise and the reaction mixture was maintained for 2 h at 5° C. and for an additional 60 min at rt. The reaction mixture was extracted with ethyl acetate (3×100 mL) and the combined organic layers were dried (magnesium sulfate) and concentrated to provide crude benzyl 3-hydroxypyrrolidine-1-carboxylate as brown oil.

### 3. Synthesis of benzyl 3-(tetrahydro-2H-pyran-2-yloxy)pyrrolidine-1-carboxylate

3,4-Dihydro-2H-pyran (226 mmol) and p-toluenesulfonic acid (2.26 mmol) were added to a solution of benzyl 3-hydroxypyrrolidine-1-carboxylate (45.2 mmol) in dichloromethane (100 mL) at 0° C. The reaction mixture was allowed to warm to rt and was maintained for 60 min. The reaction mixture was washed with sodium bicarbonate (100 mL) and brine (100 mL), dried (magnesium sulfate), and concentrated to provide benzyl 3-(tetrahydro-2H-pyran-2-yloxy)pyrrolidine-1-carboxylate in 98% yield as yellow oil.

### 4. Synthesis of 3-tetrahydro-2H-pyran-2-yloxy)pyrrolidine

The suspension of benzyl 3-(tetrahydro-2H-pyran-2-yloxy)pyrrolidine-1-carboxylate (44.3 mmol) and 10% palladium on carbon (2.3 g) in methanol (100 mL) was maintained under an atmosphere of hydrogen gas for 2 h at rt. The insoluble solids were removed by filtration and the filtrate was concentrated to provide 3-(tetrahydro-2H-pyran-2-yloxy)pyrrolidine in 67% yield as a yellow liquid.

### 5. Synthesis of 1-(3-bromophenyl)-3-(tetrahydro-2H-pyran-2-yloxy)pyrrolidine

Palladium(II) acetate (0.300 mmol), BINAP (0.890 mmol), and cesium carbonate (74.5 mmol) were added to a solution of 1,3-dibromobenzene (29.9 mmol) and 3-(tetrahydro-2H-pyran-2-yloxy)pyrrolidine (32.8 mmol) in toluene (100 mL) under an atmosphere of nitrogen and the reaction mixture was maintained for 16 h at reflux. The insoluble solids were removed by filtration and the filtrate was washed with brine (3×100 mL), dried (magnesium sulfate), and concentrated. The residue was purified by Flash chromatography (1/100 ethyl acetate/petroleum ether) to provide 1-(3-bromophenyl)-3-(tetrahydro-2H-pyran-2-yloxy)pyrrolidine in 13% yield as a yellow liquid.

### 6. Synthesis of 3-(3-(tetrahydro-2H-pyran-2-yloxy)pyrrolidin-1-yl)benzene-1-sulfonyl chloride

n-Butyllithium (5.4 mmol) was added dropwise to a solution of 1-(3-bromophenyl)-3-(tetrahydro-2H-pyran-2-yloxy)pyrrolidine (4.29 mmol) in tetrahydrofuran (50 mL) at −78° C. and the reaction mixture was maintained for 40 min. Sulfur dioxide (7.03 mmol) was added and the reaction mixture was maintained for 60 min at −78° C. The reaction mixture was diluted with hexane (50 mL) and the precipitated solids were collected by filtration. The solid was suspended in dichloromethane (50 mL) at 0° C. and N-chlorosuccinamide (6.97 mmol) was added in several batches. The reaction mixture was allowed to warm to rt and was maintained for 40 min. The reaction mixture was washed with (2 M) sodium hydrogen sulfate (3×100 mL) and brine (100 mL), was dried (magnesium sulfate), and was concentrated to provide 3-(3-(tetrahydro-2H-pyran-2-yloxy)pyrrolidin-1-yl)benzene-1-sulfonyl chloride in 61% yield as yellow oil. Data: ¹H NMR (CDCl₃) δ 7.38 (m, 1H), 7.30 (m, 1H), 7.10 (s, 1H), 6.82 (d, 1H), 4.75 (m, 1H), 4.52 (m, 1H), 3.90 (m, 1H), 3.38-3.57 (m, 5H), 2.18 (m, 1H), 2.05 (m, 1H), 1.70-1.80 (m, 2H), 1.55 (d, 4H). LC/MS (ES) m/z 417 [M+BnNH₂+H]⁺.

### Intermediate 15: Synthesis of isoquinoline-8-sulfonyl chloride

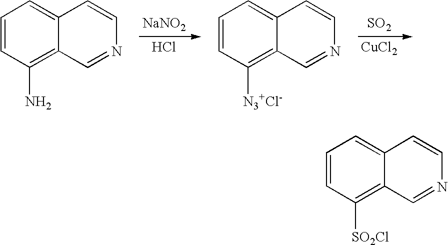

Hydrochloric acid (60.2 mmol) was added dropwise to a solution of isoquinolin-8-amine (16.1 mmol) and acetic acid (200 mmol) in acetonitrile (100 mL) at 0° C. A solution of sodium nitrite (24.2 mmol) in water (2 mL) was subsequently added and the mixture was maintained for 45 min at 0° C. Sulfur dioxide gas was passed through the reaction mixture for 2 h whereupon a solution of copper(II) chloride dihydrate (21.1 mmol) in water (5 mL) was added. Sulfur dioxide gas was passed through the reaction mixture for an additional 60 min and the reaction mixture was maintained for 16 h at 0° C. The reaction mixture was diluted with ice water (400 mL) and the resulting mixture was extracted with dichloromethane (3×200 mL). The combined organic layers were washed with brine, dried (sodium sulfate), and concentrated to provide isoquinoline-8-sulfonyl chloride in 12% yield as a brown solid. Data: LC/MS m/z 228 [M+1]⁺.

### Intermediate 16: Synthesis of 1-(2,2,2-trifluoroacetyl)-1,2,3,4-tetrahydroquinoline-6-sulfonyl chloride

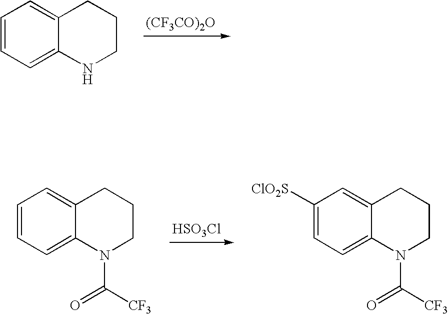

### 1. Synthesis of 1-(3,4-dihydroquinolin-1(2H)-yl)-2,2,2-trifluoroethanone

A solution of trifluoroacetic anhydride (30.0 mmol) in chloroform (30 mL) was added dropwise to a solution of 1,2,3,4-tetrahydroquinoline (20.0 mmol) in chloroform (20 mL) at 5° C. and the resulting mixture was maintained for 2 h at rt. The reaction mixture was concentrated and the residue was purified by Flash chromatography (1/10 ethyl acetate/petroleum ether) to afford 1-(3,4-dihydroquinolin-1(2H)-yl)-2,2,2-trifluoroethanone in 87% yield as a yellow liquid.

### 2. Synthesis of 1-(2,2,2-trifluoroacetyl)-1,2,3,4-tetrahydroquinoline-6-sulfonyl chloride

1-(3,4-Dihydroquinolin-1(2H)-yl)-2,2,2-trifluoroethanone (17.5 mmol) was added to sulfurochloridic acid (30 g) at 0° C. and the resulting solution was allowed to warm to rt and maintained for 16 h. The reaction mixture was diluted with iced water (100 mL) and the resulting solution was extracted with dichloromethane (3×50 mL). The combined organic layers were dried (sodium sulfate) and concentrated. The residue was purified by Flash chromatography (1/10 ethyl acetate/petroleum ether) to afford 1-(2,2,2-trifluoroacetyl)-1,2,3,4-tetrahydroquinoline-6-sulfonyl chloride in 21% yield as a white solid. ¹H NMR (CDCl₃) δ 8.01 (d, 1H), 7.89 (s, 1H), 7.87 (s, 1H), 3.91 (t, 2H), 3.01 (t, 2H), 2.16 (m, 2H).

### Intermediate 17: Synthesis of 1-methyl-1,2,3,4-tetrahydroquinoline-7-sulfonyl chloride

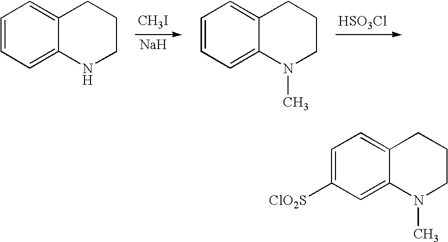

### 1. Synthesis of 1-methyl-1,2,3,4-tetrahydroquinoline

Sodium hydride (300 mmol) was added in several batches, to a solution of 1,2,3,4-tetrahydroquinoline (200 mmol) in tetrahydrofuran (150 mL) at 0-5° C. and the resulting suspension was maintained at 0-5° C. for 30 min. Iodomethane (352 mmol) was added dropwise and the reaction mixture was allowed to warm to rt and was maintained for 16 h. The mixture was filtered and the filtrate was purified by Flash chromatography (1/100 ethyl acetate/petroleum ether) to afford 1-methyl-1,2,3,4-tetrahydroquinoline in 61% yield as a yellow liquid.

### 2. Synthesis of 1-methyl-1,2,3,4-tetrahydroquinoline-7-sulfonyl chloride

A solution of 1-methyl-1,2,3,4-tetrahydroquinoline (68.0 mmol) in dichloromethane (20 mL) was added dropwise to sulfurochloridic acid (690 mmol) at 0-5° C. and the reaction mixture was allowed to warm to rt and was maintained for 16 h. The reaction mixture was diluted with iced water (300 mL) and was extracted with ethyl acetate (3×150 mL). The organic layers were combined, concentrated, and the residue was purified by Flash chromatography (1/20 ethyl acetate/petroleum ether) to afford 1-methyl-1,2,3,4-tetrahydroquinoline-7-sulfonyl chloride in 8% yield as a yellow liquid. ¹H NMR (CDCl₃) δ 7.19 (d, 1H), 7.10 (d, 1H), 7.06 (s, 1H), 3.33 (t, 2H), 2.97 (s, 3H), 2.81 (d, 2H), 1.99 (m, 2H).

### Intermediate 18: Synthesis of 1-methyl-1,2,3,4-tetrahydroquinoline-6-sulfonyl chloride

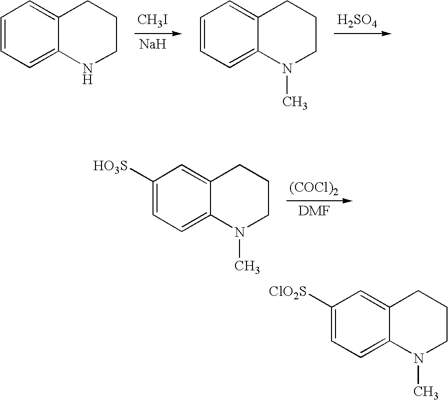

### 1. Synthesis of 1-methyl-1,2,3,4-tetrahydroquinoline-6-sulfonic acid

A solution of sulfuric acid (60.0 mmol) in ether (40 mL) was added dropwise to a solution of 1-methyl-1,2,3,4-tetrahydroquinoline (61.1 mmol) in diethylether (10 mL) at 5° C. The diethylether was decanted and the resulting solution was maintained for 3 h at 170° C. The reaction mixture was concentrated and the residue was diluted with methanol (100 mL). The precipitated solids were isolated by filtration and dried to provide 1-methyl-1,2,3,4-tetrahydroquinoline-6-sulfonic acid in 34% yield as a white solid.

### 2. Synthesis of 1-methyl-1,2,3,4-tetrahydroquinoline-6-sulfonyl chloride

Oxalyl chloride (157.6 mmol) was added dropwise at rt to a solution of 1-methyl-1,2,3,4-tetrahydroquinoline-6-sulfonic acid (22.0 mmol) in dichloromethane (100 mL) and N,N-dimethylformamide (10 mL). The resulting solution was maintained for 2 h, then was diluted with iced water (200 mL). The resulting solution was extracted with dichloromethane (2×100 mL) and the combined organics were dried (sodium sulfate), filtered and concentrated. The residue was purified by Flash chromatography (1/4 ethyl acetate/petroleum ether) to afford 1-methyl-1,2,3,4-tetrahydroquinoline-6-sulfonyl chloride in 20% yield as a yellow solid. ¹H NMR (CDCl₃) δ 7.69 (d, 1H), 7.51 (s, 1H), 6.54 (d, 1H), 3.57 (t, 2H), 3.02 (s, 3H), 2.78 (d, 2H), 1.98 (m, 2H).

### Intermediate 19: Synthesis of 2-methyl-1,2,3,4-tetrahydroisoquinoline-8-sulfonyl chloride

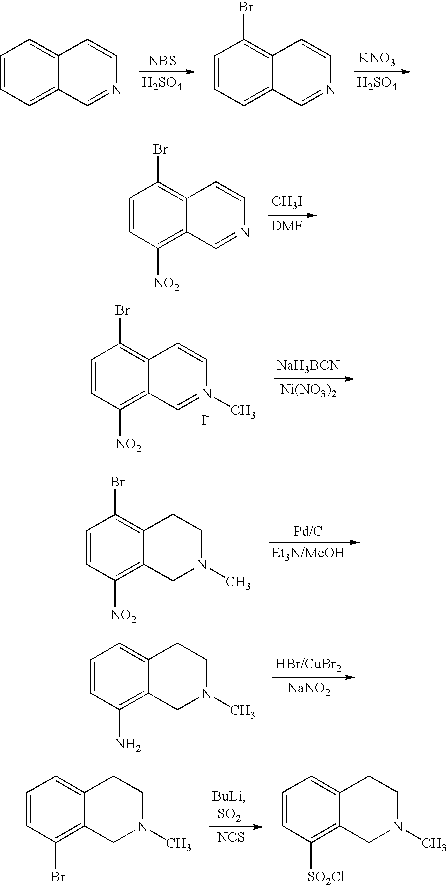

### 1. Synthesis of 5-bromoisoquinoline

Isoquinoline (132 mmol) was added in several batches to sulfuric acid (150 mL) at 0° C. The reaction mixture was cooled at −25° C. and N-bromosuccinamide (164 mmol) was added in portions and the reaction mixture was maintained for 2 h. The reaction mixture was allowed to warm to rt and was maintained for an additional 16 h. The reaction mixture was diluted with 1000 mL of ice water (1000 mL) and the pH of the solution was adjusted to 8-10 with concentrated ammonium hydroxide. The resulting solution was extracted with ethyl acetate (4×500 mL) and the combined organic layers were dried (sodium sulfate) and concentrated. The residue was purified by Flash chromatography (1/5 ethyl acetate/petroleum ether) to provide 5-bromoisoquinoline in 81% yield as a white solid.

### 2. Synthesis of 5-bromo-8-nitroisoquinoline

A solution of potassium nitrate (149 mmol) in sulfuric acid (100 mL) was added over 1 h to a solution of 5-bromoisoquinoline (107 mmol) in sulfuric acid (120 mL) at rt. The reaction mixture was maintained at rt for 1 h and was diluted with ice water (600 mL). The pH of the solution was adjusted to 8-10 with concentrated ammonium hydroxide and the precipitated solids were collected by filtration, washed with water (2×500 mL), and dried in a vacuum oven to provide 5-bromo-8-nitroisoquinoline in 90% yield as a yellow solid.

### 3. Synthesis of 5-bromo-8-nitro-N-methylisoquinolinium iodide

Iodomethane (506 mmol) was added to a solution of 5-bromo-8-nitroisoquinoline (101 mmol) in N,N-dimethylformamide (200 mL) and the reaction mixture was maintained for 16 h at 40° C. The precipitated solids were collected by filtration, washed with ether (2×250 mL), and dried to provide 5-bromo-8-nitro-N-methylisoquinolinium iodide in 83% yield as a red solid.

### 4. Synthesis of 5-bromo-2-methyl-8-nitro-1,2,3,4-tetrahydroisoquinoline

Sodium cyanoborohydride (169 mmol) was added in several batches to a solution of 5-bromo-8-nitro-N-methylisoquinolinium iodide (84.4 mmol) and nickel(II) nitrate hexahydrate (43.3 mmol) in methanol (200 mL) and the reaction mixture was maintained for 5 h at rt. The reaction mixture was concentrated and the residue was dissolved with 800 mL of water. The pH of the aqueous layer was adjusted to 8-10 was accomplished by the addition of 5% sodium hydroxide and the insoluble solids were removed by filtration. The resulting solution was extracted with ethyl acetate (2×800 mL) and the combined organic layers were dried (sodium sulfate) and concentrated. The residue was purified by Flash chromatography (1/5 ethyl acetate/petroleum ether) to provide 5-bromo-2-methyl-8-nitro-1,2,3,4-tetrahydroisoquinoline in 83% yield as a yellow solid.

### 5. Synthesis of 2-methyl-1,2,3,4-tetrahydroisoquinolin-8-amine

A suspension of 5-bromo-2-methyl-8-nitro-1,2,3,4-tetrahydroisoquinoline (17.9 mmol) and 10% palladium on carbon (4.5 g) in methanol (150 mL) and triethylamine (15 mL) was maintained under an atmosphere of hydrogen gas for 3 h at rt. The insoluble solids were removed by filtration and the filtrate was concentrated. The residue was diluted with 10% sodium carbonate (50 mL) and was extracted with ethyl acetate (4×50 mL) and the combined organic layers were dried (sodium sulfate) and concentrated. The residue was purified by Flash chromatography (50/1 dichloromethane/methanol) to provide 2-methyl-1,2,3,4-tetrahydroisoquinolin-8-amine in 89% yield as a light yellow oil.

### 6. Synthesis of 8-bromo-2-methyl-1,2,3,4-tetrahydroisoquinoline

Sodium nitrite (3.33 mmol) was added in several batches to a solution of 2-methyl-1,2,3,4-tetrahydroisoquinolin-8-amine (3.08 mmol) in concentrated hydrobromic acid (5 mL) and water (5 mL) at 0° C. and the mixture was maintained for 30 min. Copper(I) bromide (3.83 mmol) was added to 3 M hydrobromic acid (10 mL) in a second reaction vessel at 0° C. under an atmosphere of nitrogen and the mixture was maintained for 10 min. The contents of the diazotization reaction were added dropwise to the copper solution and the reaction mixture was maintained for 30 min at 0° C. The pH of the aqueous layer was adjusted to 9 with 10% sodium hydroxide and the resulting solution was extracted with dichloromethane (3×50 mL). The combined organic layers were dried (potassium carbonate), filtered, and concentrated. The residue was purified by Flash chromatography (1/1 ethyl acetate/petroleum ether) to provide 8-bromo-2-methyl-1,2,3,4-tetrahydroisoquinoline in 65% yield as light yellow oil.

### 7. Synthesis of 2-methyl-1,2,3,4-tetrahydroisoquinoline-8-sulfonyl chloride

A 2.5 M solution of n-butyllithium in hexane (17 mmol) was added over 15 min to a solution of 8-bromo-2-methyl-1,2,3,4-tetrahydroisoquinoline (13.3 mmol) in tetrahydrofuran (30 mL) at −78° C. and the reaction mixture was maintained for 40 min. The reaction mixture was cooled to −100° C. and sulfur dioxide (13.9 mmol) was added. The reaction mixture was allowed to warm to −78° C. and was maintained for 20 min. The reaction mixture was allowed to warm to rt and was maintained for an additional 60 min. The reaction mixture was diluted with n-hexane (60 mL) and the resultant light yellow solid was isolated by filtration. The solid was dissolved in dichloromethane (80 mL), cooled to −10° C., and was treated with N-chlorosuccinamide (20.2 mmol) in several portions. The reaction mixture was allowed to warm to rt and was maintained for 60 min. The reaction mixture was washed with saturated sodium hydrogen sulfate (2×100 mL) and brine (2×50 mL), was dried (sodium sulfate), and was concentrated to provide 2-methyl-1,2,3,4-tetrahydroisoquinoline-8-sulfonyl chloride in 44% yield as a light yellow solid. Data: ¹H NMR (DMSO-d₆) δ 7.63 (d, 1H), 7.22 (m, 2H), 5.03 (d, 1H), 4.40 (m, 1H), 3.60 (d, 1H), 3.34 (d, 1H), 2.94 (m, 2H), 2.49 (s, 3H). LC/MS (ES) m/z 246 [M+1]⁺.

### Intermediate 20: Synthesis of 2-oxo-1,2-dihydroquinoline-5-sulfonyl chloride

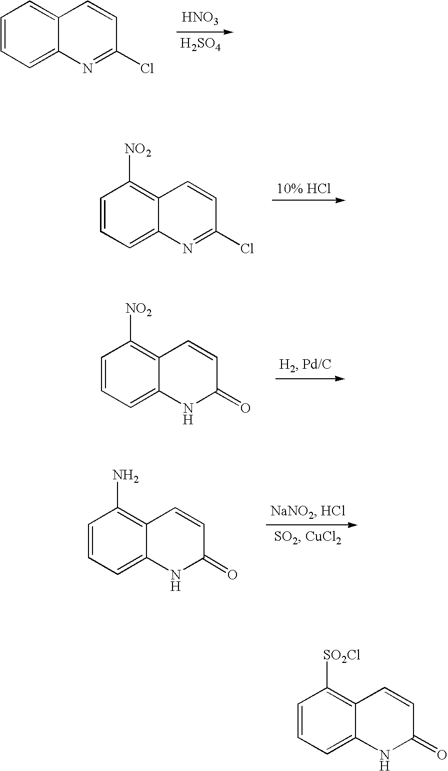

### 1. Synthesis of 2-chloro-5-nitroquinoline

A solution of nitric acid (16 mL) and sulfuric acid (8 mL) was added over period of 20 min to a solution of 2-chloroquinoline (61.1 mmol) in sulfuric acid (150 mL) at 0° C. The reaction mixture was heated at 40° C. for 30 min and was quenched with ice water (800 mL). The precipitated solids were collected by filtration and purified by Flash chromatography (20/1 petroleum ether/ethyl acetate) to provide 2-chloro-5-nitroquinoline in 19% yield as a yellow solid.

### 2. Synthesis of 5-nitro-2-oxo-1,2-dihydroquinoline

A solution of 2-chloro-5-nitroquinoline (1.92 mmol) in 10% hydrochloric acid (50 mL) was heated at reflux for 16 h. The insoluble solids were removed by filtration and the filtrate was extracted with ethyl acetate (5×100 mL). The combined organic layers were washed with brine (50 mL) and concentrated to provide 5-nitro-2-oxo-1,2-dihydroquinoline in 82% yield as a yellow solid.

### 3. Synthesis of 5-amino-2-oxo-1,2-dihydroquinoline

A suspension of 5-nitro-2-oxo-1,2-dihydroquinoline (36.8 mmol) and 10% palladium on carbon (1 g) in N,N-dimethylformamide (250 mL) was maintained under an atmosphere of hydrogen gas at 35° C. for 16 h. The insoluble solids were removed by filtration, washed with methanol (2×5 mL), and concentrated to provide 5-amino-2-oxo-1,2-dihydroquinoline in 92% yield as a white solid.

### 4. Synthesis of 2-oxo-1,2-dihydroquinoline-5-sulfonyl chloride

Hydrochloric acid (12 mL) was added to a solution of 5-amino-2-oxo-1,2-dihydroquinoline (21.9 mmol) in acetic acid (18 mL) and acetonitrile (80 mL) at 0° C. Solid sodium nitrite (26.2 mmol) was subsequently added and the mixture was maintained for 60 min at 0° C. Sulfur dioxide gas was bubbled through the solution for 2 h while the temperature was maintained at 0° C. Solid copper(II) chloride dihydrate (23.5 mmol) was added in portions and sulfur dioxide gas was bubbled through the solution for an additional 60 min. The reaction mixture was allowed to warm to rt and was maintained for 16 h. The reaction mixture was diluted with ice water (250 mL) and was extracted with ethyl acetate (4×100 mL). The combined organic layers were washed with brine (4×300 mL), dried (sodium sulfate), and concentrated to provide 2-oxo-1,2-dihydroquinoline-5-sulfonyl chloride in 14% yield as a yellow solid. Data: ¹H NMR (DMSO-d₆) δ 8.73 (d, 1H), 7.51 (d, 1H), 7.42 (d, 1H), 7.30 (m, 1H), 6.52 (d, 1H). LC/MS (ES) m/z 245 [M+1]⁺.

### Intermediate 21: Synthesis of 2-oxo-1,2-dihydroquinoline-6-sulfonyl chloride

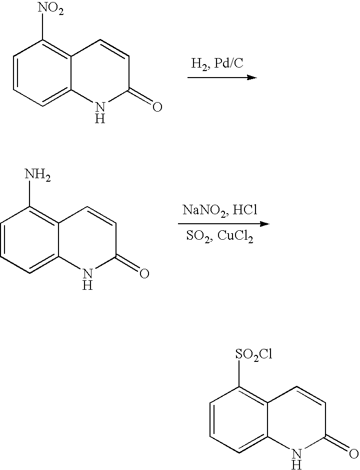

### 1. Synthesis of 6-aminoquinolin-2(1H)-one

A suspension of 6-nitroquinolin-2(1H) one (52.6 mmol) and 10% palladium on carbon (8.6 g) in N,N-dimethylformamide (200 mL) was maintained under an atmosphere of hydrogen gas at rt for 16 h. The insoluble solids were removed by filtration and the filtrate was concentrated. The residue was diluted with water (100 mL) and the precipitated solids were collected by filtration. The solids were washed with water (10 mL) and hexane (10 mL), and dried to provide 6-aminoquinolin-2(1H)-one in 90% yield as a gray solid.

### 2. Synthesis of 2-oxo-1,2-dihydroquinoline-6-sulfonyl chloride

Hydrochloric acid (7 mL) was added to a solution of 6-aminoquinolin-2(1H)-one (12 mmol) in acetic acid (15 mL) and acetonitrile (150 mL) at 0° C. A solution of sodium nitrite (16.0 mmol) in water (1 mL) was subsequently added dropwise and the mixture was maintained for 30 min at 0° C. Sulfur dioxide gas was bubbled through the solution for 2 h while the temperature was maintained at 0° C. A solution of copper(II) chloride dihydrate (12.9 mmol) in water (2 mL) was added dropwise and sulfur dioxide gas was bubbled through the solution for an additional 60 min. The reaction mixture was allowed to warm to rt and was maintained for 16 h The reaction mixture was diluted with ice water (100 mL) and was extracted with dichloromethane (2×1000 mL). The combined organic layers were washed with brine (300 mL), dried (sodium sulfate), and concentrated. The residue was triturated with hexane (10 mL) to provide 2-oxo-1,2-dihydroquinoline-6-sulfonyl chloride in 4% yield as a gray solid. Data: ¹H NMR (CDCl₃) δ 11.80 (s, 1H), 7.95 (m, 2H), 7.72 (d, 1H), 7.25 (d, 1H), 6.48 (d, 1H). LC/MS (ES) m/z 308 [M+C5H11N2+H-Cl]⁺.

### Intermediate 22: Synthesis of 2-oxo-1,2-dihydroquinoline-7-sulfonyl chloride

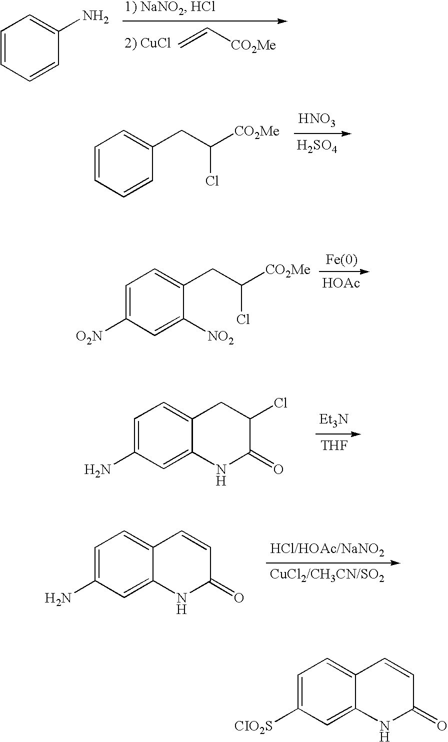

### 1. Synthesis of methyl 2-chloro-3-phenylpropanoate

Into a 250 ml 3-necked roundbottom flask, was placed a solution of aniline (50.0 mmol) in acetone (100 mL). To the mixture was added concentrated hydrochloric acid (20 mL). This was followed by the addition of a solution of sodium nitrite (50.7 mmol) in water (10 mL), which was added dropwise with stirring, while cooling to a temperature of 0-10° C. The mixture was allowed to react, with stirring, for 1 h while maintained at 10 degree C. To the above was added methyl acrylate (500 mmol) dropwise with stirring, while cooling to a temperature of 0-10° C. To the above was added copper(I) chloride (3.03 mmol) in several batches, while cooling to a temperature of 0° C. The resulting solution was allowed to react, with stirring, for 1 h while the temperature was maintained at rt. The resulting solution was extracted three times with 100 ml of ether dried (sodium sulfate), and concentrated. The residue was purified by Flash chromatography (100/0 to 50/1 petroleum ether/ethyl acetate) to provide methyl 2-chloro-3-phenylpropanoate in 66% yield as a yellow liquid.

### 2. Synthesis of methyl 2-chloro-3-(2,4-dinitrophenyl)propanoate

Into a 50 ml 3-necked roundbottom flask, was placed a solution of methyl 2-chloro-3-phenylpropanoate (10.1 mmol) in sulfuric acid (3 mL). This was followed by the addition of a solution of nitric acid (49.8 mmol) in sulfuric acid (3 mL), which was added dropwise with stirring, while cooling to a temperature of 0-20° C. The resulting solution was allowed to react, with stirring, for 60 min while the temperature was maintained at 20° C. The reaction mixture was then quenched by the adding ice water (100 mL). The resulting solution was extracted three times with 100 ml of ethyl acetate (3×100 mL) and the organic layers combined and dried (sodium sulfate). The residue was purified by Flash chromatography (50/1 petroleum ether/ethyl acetate) to provide methyl 2-chloro-3-(2,4-dinitrophenyl)propanoate in 65% yield as a yellow solid.

### 3. Synthesis of 7-amino-3-chloro-3,4-dihydroquinolin-2(1H)-one

Iron powder (229 mmol) was added in several portions to a solution of methyl 2-chloro-3-(2,4-dinitrophenyl)propanoate (27.7 mmol) in acetic acid (75 mL) and water (5 mL) at 50° C. The reaction mixture was maintained for 2 h at 50° C. and was allowed to cool to rt. The resulting solution was diluted with ethyl acetate (100 mL) and the precipitated solids were removed by filtration (5×200 mL ethyl acetate wash). The combined organic layers were washed with water (5×500 mL), dried (sodium sulfate), and concentrated to provide 7-amino-3-chloro-3,4-dihydroquinolin-2(1)-one in 40% yield as a light yellow solid.

### 4. Synthesis of 7-aminoquinolin-2(1H)-one

Triethylamine (50.5 mmol) was added to a solution of 7-amino-3-chloro-3,4-dihydroquinolin-2(1H)-one (10.2 mmol) in tetrahydrofuran (120 mL) and the reaction mixture was heated at reflux for 18 h. The precipitated solids were collected by filtration, washed with water (5×50 mL), and dried in a vacuum oven to provide 7-aminoquinolin-2(1H)-one in 68% yield as a white solid.

### 5. Synthesis of 2-oxo-1,2-dihydroquinoline-7-sulfonyl chloride

Hydrochloric acid (3.24 g) was added dropwise to a solution of 7-aminoquinolin-2(1H)-one (6.25 mmol) and acetic acid (5.0 g) in acetonitrile (100 mL) at 0° C. A solution of sodium nitrite (7.54 mmol) in water (0.5 mL) was subsequently added and the mixture was maintained for 30 min at 0° C. Sulfur dioxide gas was passed through the reaction mixture for 2 h whereupon a solution of copper(II) chloride dihydrate (6.22 mmol) in water (0.5 mL) was added dropwise. Sulfur dioxide gas was passed through the reaction mixture for an additional 2 h and the reaction mixture was maintained for an additional 2 h at 0° C. The reaction mixture was diluted with ice water (20 mL) and the resulting mixture was extracted with dichloromethane (2×200 mL). The combined organic layers were washed with water (3×100 mL) and brine (5×100 mL), dried (sodium sulfate) and concentrated. The residue was triturated with hexane and dried under high vacuum to provide 2-oxo-1,2-dihydroquinoline-7-sulfonyl chloride in 55% yield as a yellow solid. Data: ¹H NMR (DMSO-d₆) δ 7.86 (d, 1H), 7.61 (d, 3H), 7.36 (d, 1H), 6.47 (d, 1H). LC/MS (ES) m/z 308 [M+C5H11N2+H-Cl]⁺.

### Intermediate 23: Synthesis of 2-oxo-1,2-dihydroquinoline-8-sulfonyl chloride

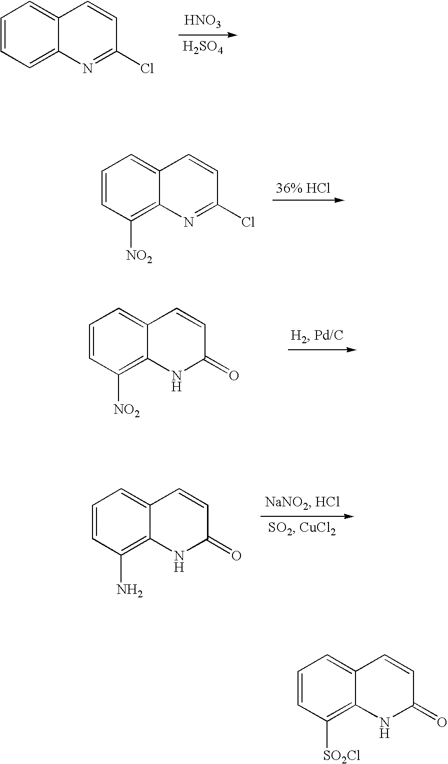

### 1. Synthesis of 2-chloro-8-nitroquinoline

A solution of nitric acid (16 mL) and sulfuric acid (8 mL) was added over period of 20 min to a solution of 2-chloroquinoline (61.1 mmol) in sulfuric acid (150 mL) at 0° C. The reaction mixture was heated at 40° C. for 30 min and was quenched with ice water (800 mL). The precipitated solids were collected by filtration and purified by Flash chromatography (20/1 petroleum ether/ethyl acetate) to provide 2-chloro-8-nitroquinoline in 64% yield as a yellow solid.

### 2. Synthesis of 8-nitroquinolin-2(1H)-one

A solution of 2-chloro-8-nitroquinoline (28.8 mmol) in concentrated hydrochloric acid (30 mL) was heated at reflux for 16 h. The precipitated solids were collected by filtration and dried to provide 8-nitroquinolin-2(1H)-one in 58% yield as a yellow solid.

### 3. Synthesis of 8-aminoquinolin-2(1H)-one

A suspension of 8-nitroquinolin-2(1H)-one (10.5 mmol) and 10% palladium on carbon (600 mg) in methanol (25 mL) was maintained under an atmosphere of hydrogen gas at rt for 3 h. The insoluble solids were removed by filtration, washed with methanol (2×5 mL), and concentrated to provide 8-aminoquinolin-2(1H)-one in 53% yield as a white solid.

### 4. Synthesis of 2-oxo-1,2-dihydroquinoline-8-sulfonyl chloride

Hydrochloric acid (12 mL) was added to a solution of 8-aminoquinolin-2(1H)-one (21.9 mmol) in acetic acid (18 mL) and acetonitrile (80 mL) at 0° C. Solid sodium nitrite (26.2 mmol) was subsequently added and the mixture was maintained for 60 min at 0° C. Sulfur dioxide gas was bubbled through the solution for 2 h while the temperature was maintained at 0° C. Solid copper(II) chloride dihydrate (23.5 mmol) was added in portions and sulfur dioxide gas was bubbled through the solution for an additional 60 min. The reaction mixture was allowed to warm to rt and was maintained for 16 h. The reaction mixture was diluted with ice water (250 mL) and was extracted with ethyl acetate (4×100 mL). The combined organic layers were washed with brine (4×300 mL), dried (sodium sulfate), and concentrated to provide 2-oxo-1,2-dihydroquinoline-8-sulfonyl chloride in 35% yield as a yellow solid. Data: ¹H NMR (DMSO-d₆) δ 7.97 (d, 1H), 7.82 (d, 1H), 7.69 (d, 1H), 7.19 (t, 1H), 6.55 (d, 1H). LC/MS (ES) m/z 244 [M+1]⁺.

### Intermediate 24. Synthesis of 2-oxo-1,2,3,4-tetrahydroquinoline-5-sulfonyl chloride

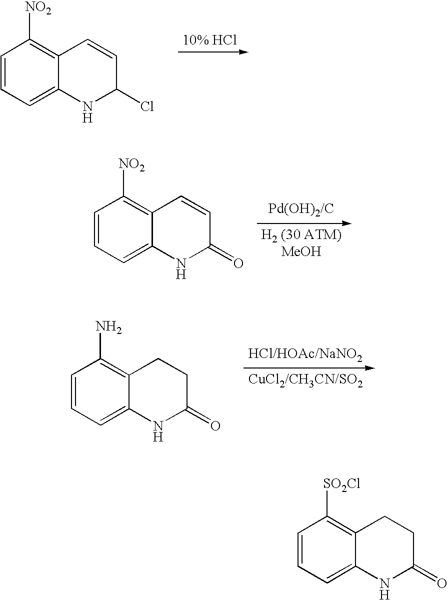

### 1. Synthesis of 5-nitroquinolin-2(1H)-one

2-Chloro-5-nitro-1,2-dihydroquinoline (0.95 mmol) was diluted with 10% hydrochloric acid (20 mL) and the reaction mixture was heated at reflux for 16 h. The insoluble solids were removed by filtration and the filtrate was extracted with ethyl acetate (3×300 mL). The combined organic extracts were dried (sodium sulfate) and concentrated to provide 5-nitroquinolin-2(1H)-one in 100% yield as a yellow solid.

### 2. Synthesis of 5-amino-3,4-dihydroquinolin-2(1H)-one

A suspension of 5-nitroquinolin-2(1H)-one (12.6 mmol) and 10% palladium(II) hydroxide on carbon (1 g) in methanol (100 mL) was maintained under an atmosphere of hydrogen gas (30 ATM) for 48 h at rt. The insoluble solids were removed by filtration and the filtrate was concentrated to provide 5-amino-3,4-dihydroquinolin-2(1H)-one in 53% yield as a yellow solid.

### 3. Synthesis of 2-oxo-1,2,3,4-tetrahydroquinoline-5-sulfonyl chloride

Hydrochloric acid (7.1 g) was added dropwise to a solution of 5-amino-3,4-dihydroquinolin-2(1H)-one (13.6 mmol) and acetic acid (11 g) in acetonitrile (120 mL) at 0° C. A solution of sodium nitrite (16.4 mmol) in water (1 mL) was subsequently added and the mixture was maintained for 30 min at 0° C. Sulfur dioxide gas was passed through the reaction mixture for 2 h whereupon solid copper(II) chloride dihydrate (13.6 mmol) was added in portions. Sulfur dioxide gas was passed through the reaction mixture for an additional 30 min. The reaction mixture was allowed to warm to rt and was maintained for 16 h. The reaction mixture was diluted with ice water (100 mL) and the resulting mixture was extracted with diethyl ether (3×300 mL). The combined organic layers were dried (sodium sulfate) and concentrated. The residue was purified by Flash chromatography (10/1 petroleum ether/ethyl acetate) to provide 2-oxo-1,2,3,4-tetrahydroquinoline-5-sulfonyl chloride in 25% yield as a yellow solid, Data: ¹H-NMR (CDCl₃) δ 9.11 (s, 1H), 7.87 (d, 1H), 7.43 (t, 1H), 7.26 (d, 1H), 2.75 (t, 2H), 2.54 (t, 2H). LC/MS (ES) m/z 310 [M+H]⁺.

### Intermediate 25: Synthesis of 2-oxo-1,2,3,4-tetrahydroquinoline-7-sulfonyl chloride

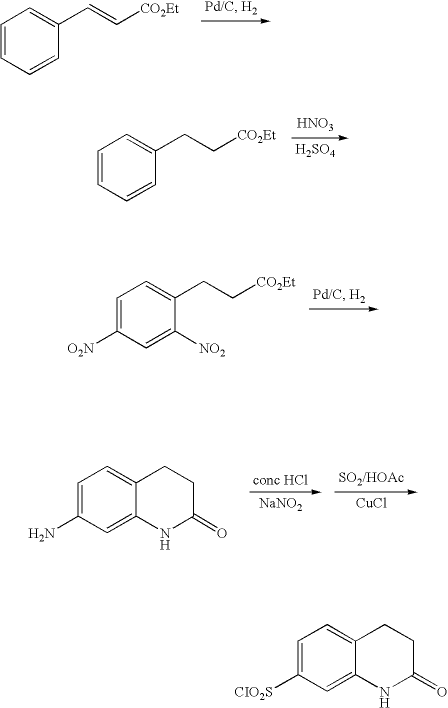

### 1. Synthesis of ethyl 3-phenylpropanoate

A suspension of ethyl cinnamate (56.8 mmol) and 10% palladium on carbon (2 g) in methanol (200 mL) was maintained under an atmosphere of hydrogen gas for 16 h at 35° C. The insoluble solids were removed by filtration and the filtrate was concentrated to provide ethyl 3-phenylpropanoate in 99% yield as a colorless oil.

### 2. Synthesis of ethyl 3-(2,4-dinitrophenyl)propanoate

Ethyl 3-phenylpropanoate (28.1 mmol) was added to a mixture of fuming nitric acid (25 mL) in concentrated sulfuric acid (50 mL) at 0° C. and the reaction mixture was maintained for 60 min. The reaction mixture was then heated at 60° C. for 16 h, allowed to cool to rt, and was diluted with ice water. The resulting solution was extracted with ethyl acetate (2×50 mL) and the combined organic layers were washed with sodium bicarbonate (2×50 mL), dried (magnesium sulfate), and concentrated to provide ethyl 3-(2,4-dinitrophenyl)propanoate in 27% yield as a yellow solid.

### 3. Synthesis of 7-amino-3,4-dihydroquinolin-2(1H)-one

A suspension of ethyl 3-(2,4-dinitrophenyl)propanoate (5.60 mmol) and 10% palladium on carbon (0.5 g) in methanol (20 mL) was maintained under an atmosphere of hydrogen gas for 16 h at 30° C. The insoluble solids were removed by filtration and the filtrate was concentrated to provide 7-amino-3,4-dihydroquinolin-2(1H)-one in 55% yield as a green-yellow solid.

### 4. Synthesis of 2-oxo-1,2,3,4-tetrahydroquinoline-7-sulfonyl chloride

A solution of sodium nitrite (2.90 mmol) in water (2 mL) was added to a solution of 7-amino-3,4-dihydroquinolin-2(1H)-one (2.16 mmol) in conc.hydrochloric acid (6 mL) at 0° C. and the reaction mixture was maintained for 30 min. In a separate reaction vessel, sulfur dioxide gas was passed through acetic acid (10 mL) at rt until the solution was saturated. Copper(I) chloride (2.02 mmol) was added and was followed by the amine solution and the reaction mixture was maintained for 60 min. The reaction mixture was diluted with ice water and was extracted with ethyl acetate (2×20 mL). The combined organic layers were washed with water (2×10 mL) and saturated sodium bicarbonate (10 mL), dried (sodium sulfate), and concentrated to provide 2-oxo-1,2,3,4-tetrahydroquinoline-7-sulfonyl chloride in 45% yield as a brown solid. Data: ¹HNMR (CDCl₃) δ 2.89 (m, 2H), 2.95 (m, 2H), 7.41 (m, 1H), 7.43 (m, 1H), 7.47 (m, 1H). LC/MS (ES) m/z 315 [M-1]⁻

### Intermediate 26. Synthesis of 3-oxo-3,4-dihydro-2H-benzo[b][1,4]oxazine-8-sulfonyl chloride

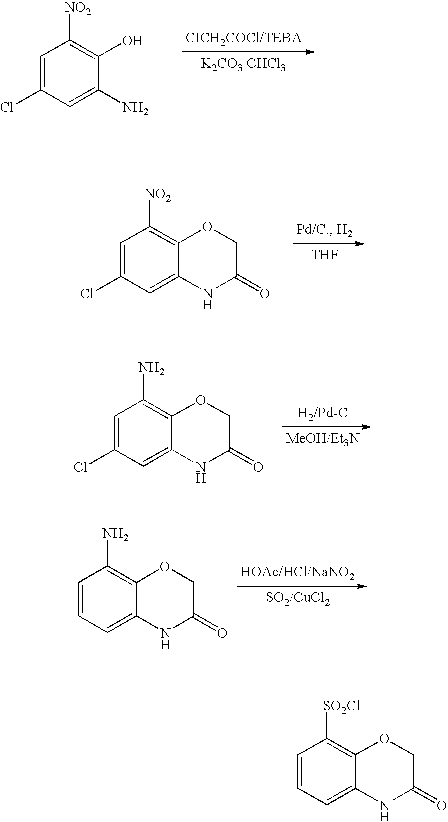

### 1. Synthesis of 6-chloro-8-nitro-2H-benzo[b][1,4]oxazin-3(4H)-one

A solution of 2-chloroacetyl chloride (255 mmol) in chloroform (200 mL) was added over 45 min to a suspension of 2-amino-4-chloro-6-nitrophenol (212 mmol), N-benzyl-N-chloro-N,N-diethylethanamine (TEBA) (130 mmol), and potassium carbonate (638 mmol) in chloroform (2.50 L) at 0° C. The reaction mixture was maintained at 0° C. for 60 min and was then heated at 55° C. for 16 h. The insoluble solids were removed by filtration and the filtrate was concentrated. The residue was diluted with water (500 mL) and the precipitated solids were collected by filtration, washed with water (3×200 mL), and dried under high vacuum. The final product was recrystallized from ethanol to provide 6-chloro-8-nitro-2H-benzo[b][1,4]oxazin-3(4H)-one in 72% yield as a brown solid.

### 2. Synthesis of 8-amino-6-chloro-2H-benzo[b][1,4]oxazin-3(4H)-one

A suspension of 6-chloro-8-nitro-2H-benzo[b][1,4]oxazin-3(4M)-one (35.0 mmol) and 10% palladium on carbon (3 g) in tetrahydrofuran (700 mL) was maintained under an atmosphere of hydrogen at 35° C. for 4 h. The insoluble solids were removed by filtration and the filtrate was concentrated to provide 8-amino-6-chloro-2H-benzo[b][1,4]oxazin-3(4H)-one in 92% yield as a brown solid.

### 3. Synthesis of 8-amino-2H-benzo[b][1,4]oxazin-3(4H)-one

A suspension of 8-amino-6-chloro-2H-benzo[b][1,4]oxazin-3(4H)-one (9.57 mmol) and 10% palladium on carbon (1 g) in methanol (50 mL) and triethylamine (29.7 mmol) was maintained under an atmosphere of hydrogen at rt for 3 h. The insoluble solids were removed by filtration and the filtrate was concentrated to provide 8-amino-2H-benzo[b][1,4]oxazin-3(4H)-one in 64% yield as a white solid. Data: ¹H NMR (DMSO-d₆) δ 10.46 (s, 1H), 6.63 (m, 1H), 6.33 (d, 1H), 6.13 (d, 1H), 5.00 (s, 2H), 4.52 (s, 2H).

### 4. Synthesis of 3-oxo-3,4-dihydro-2H-benzo[b][1,4]oxazine-8-sulfonyl chloride

Hydrochloric acid (267 mmol) was added dropwise to a solution of 8-amino-2H-benzo[b][1,4]oxazin-3(4H)-one (50.6 mmol) and acetic acid (696 mmol) in acetonitrile (350 mL) at 0° C. A solution of sodium nitrite (61.5 mmol) in water (5 mL) was subsequently added and the mixture was maintained for 30 min at 0° C. Sulfur dioxide gas was passed through the reaction mixture for 2 h whereupon solid copper(II) chloride dihydrate (51.2 mmol) was added in portions. Sulfur dioxide gas was passed through the reaction mixture for an additional 3 h and the reaction mixture was maintained for 16 h between 0 and 10° C. The reaction mixture was diluted with ice water (200 mL) and the resulting mixture was extracted with dichloromethane (3×1.00 L). The combined organic layers were dried (sodium sulfate) and concentrated. The residue was purified by Flash chromatography (1/15 to 1/1 ethyl acetate/petroleum ether) to provide 3-oxo-3,4-dihydro-2H-benzo[b][1,4]oxazine-8-sulfonyl chloride in 16% yield as a yellow solid. Data: ¹H NMR (DMSO-d₆) δ 10.67 (s, 1H), 7.27 (m, 1H), 6.85 (m, 2H,), 4.50 (s, 2H). LC/MS (ES) m/z 312 [M+1]⁺.

### Intermediate 27: Synthesis of 3-oxo-3,4-dihydro-2H-benzo[b][1,4]oxazine-6-sulfonyl chloride

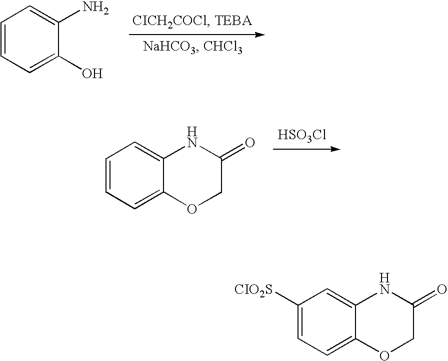

### 1. Synthesis of 2H-benzo[b][1,4]oxazin-3(4H)-one

A solution of 2-chloroacetyl chloride (72.2 mmol) in chloroform (5 mL) was added over 20 min to a suspension of 2-aminophenol (50.0 mmol), TEBA (50.0 mmol), and sodium bicarbonate (200 mmol) in chloroform (30 mL) at 0° C. The reaction mixture was maintained for 1 h and then was heated at 55° C. for 16 h. The reaction mixture was concentrated and was diluted with water. The precipitated solids were collected by filtration, washed with water (2×50 mL), and was dried under high vacuum. The final product was purified by recrystallization from ethanol to provide 2H-benzo[b][1,4]oxazin-3(4H)-one in 60% yield as a white solid.

### 2. Synthesis of 3-oxo-3,4-dihydro-2H-benzo[b][1,4]oxazine-6-sulfonyl chloride

2H-Benzo[b][1,4]oxazin-3(4H)-one (13.4 mmol) was added in several batches over 20 min to sulfurochloridic acid (10 mL) at 0° C. and the reaction mixture was maintained for 1 h. The reaction mixture was cautiously poured into ice (100 g) and the resulting mixture was extracted with dichloromethane (100 mL). The organic layer was dried (sodium sulfate) and concentrated to provide 3-oxo-3,4-dihydro-2H-benzo[b][1,4]oxazine-6-sulfonyl chloride in 66% yield as a white solid. Data: ¹HNMR (400 MHz, CDCl₃) δ 9.29 (s, 1H), 7.71 (d, 2H), 7.52 (s, 1H), 7.16 (d, 2H), 4.80 (s, 2H). LC/MS (ES) m/z 317 [M+BnNH−H].

### Intermediate 28: Synthesis of 3-oxo-3,4-dihydro-2H-benzo[b][1,4]oxazine-5-sulfonyl chloride

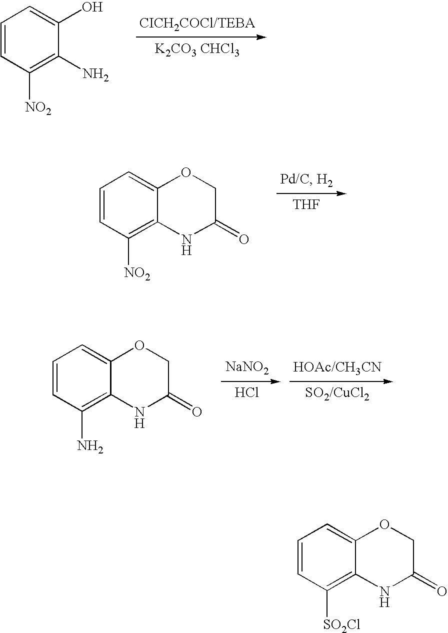

### 1. Synthesis of 5-nitro-2H-benzo[b][1,4]oxazin-3(4H)-one

A solution of 2-chloroacetyl chloride (156 mmol) in chloroform (200 mL) was added over 45 min to a suspension of 2-amino-3-nitrophenol (130 mmol), TEBA (130 mmol), and potassium carbonate (390 mmol) in chloroform (800 mL) at 0° C. The reaction mixture was maintained at 0° C. for 60 min and was then heated at 65° C. for 16 h. The insoluble solids were removed by filtration and the filtrate was concentrated. The residue was diluted with water (100 mL) and the precipitated solids were collected by filtration, washed with water (3×200 mL), and dried under high vacuum. The final product was recrystallized from ethanol to provide 5-nitro-2H-benzo[b][1,4]oxazin-3(4H)-one in 64% yield as a yellow solid.

### 2. Synthesis of 5-amino-2H-benzo[b][1,4]oxazin-3(4H)-one

A suspension of 5-nitro-2H-benzo[b][1,4]oxazin-3(4H)-one (32.5 mmol) and 10% palladium on carbon (3 g) in tetrahydrofuran (300 mL) was maintained under an atmosphere of hydrogen for 16 h. The insoluble solids were removed by filtration and the filtrate was concentrated. The residue was diluted with water (100 mL) and the precipitated solids were collected by filtration, washed with water (3×100 mL) and ether (3×100 mL), and dried to provide 5-amino-2H-benzo[b][1,4]oxazin-3(4H)-one in 100% yield as a light yellow solid.

### 3. Synthesis of 3-oxo-3,4-dihydro-2H-benzo[b][1,4]oxazine-5-sulfonyl chloride

Hydrochloric acid (16.2 g) was added dropwise to a solution of 5-amino-2H-benzo[b][1,4]oxazin-3(4H)-one (29.0 mmol) and acetic acid (24.9 g) in acetonitrile (300 mL) at 0° C. A solution of sodium nitrite (36.5 mmol) in water (2 mL) was subsequently added and the mixture was maintained for 30 min at 0° C. Sulfur dioxide gas was passed through the reaction mixture for 2 h whereupon a solution of copper(II) chloride dihydrate (30.0 mmol) in water (5 mL) was added. Sulfur dioxide gas was passed through the reaction mixture for an additional 2 h. The reaction mixture was allowed to warm to rt and was maintained for 16 h. The reaction mixture was diluted with ice water (200 mL) and the resulting mixture was extracted with dichloromethane (3×300 mL). The combined organic layers were washed with brine (5×200 mL), dried (magnesium sulfate), and concentrated. The residue was purified by Flash chromatography (1/15 ethyl acetate/petroleum ether) to provide 3-oxo-3,4-dihydro-2H-benzo[b][1,4]oxazine-5-sulfonyl chloride in 11% yield as a light yellow solid. Data: ¹H NMR (CDCl₃) δ 9.06 (s, 1H), 7.69 (d, 1H), 7.36 (m, 1H), 7.18 (d, 1H), 4.75 (s, 2H). LC/MS (ES) m/z 312 [M+C5H11N2-Cl]⁺.

### Intermediate 29: Synthesis of 3-oxo-3,4-dihydro-2H-benzo[b][1,4]oxazine-7-sulfonyl chloride

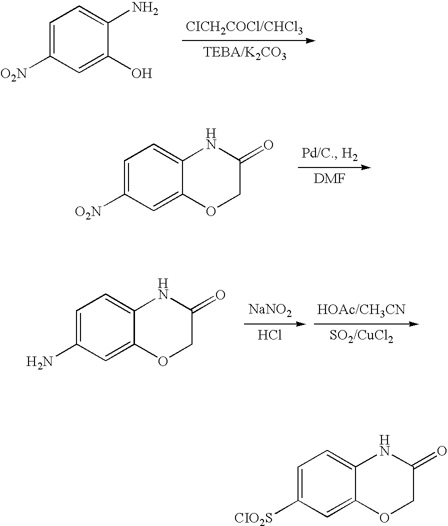

### 1. Synthesis of 7-amino-2H-benzo[b][1,4]oxazin-3(4H)-one

The suspension of 7-nitro-2H-benzo[b][1,4]oxazin-3(4H)-one (61.9 mmol) and 10% palladium on carbon (5 g) in N,N-dimethylformamide (150 mL) was maintained under an atmosphere of hydrogen gas at rt for 16 h. The insoluble solids were removed by filtration and the filtrate was concentrated. The residue was diluted water and the precipitated solids were collected by filtration, washed with hexane, and dried to provide 7-amino-2H-benzo[b][1,4]oxazin-3(4H)-one in 68% yield as a yellow solid.

### 2. Synthesis of 3-oxo-3,4-dihydro-2H-benzo[b][1,4]oxazine-7-sulfonyl chloride

Hydrochloric acid (16.2 g) was added dropwise to a solution of 7-amino-2H-benzo[b][1,4]oxazin-3(4H)-one (29.0 mmol) and acetic acid (24.9 g) in acetonitrile (200 mL) at 0° C. A solution of sodium nitrite (36.5 mmol) in water (2 mL) was subsequently added dropwise and the reaction mixture was maintained for 30 min at 0° C. Sulfur dioxide gas was passed through the reaction mixture at 0° C. for 2 h whereupon solid copper(II) chloride dihydrate (30.0 mmol) was added. Sulfur dioxide gas was passed through the reaction mixture for an additional 2 h and the reaction mixture was allowed to warm to rt and was maintained for 16 h. The reaction mixture was diluted with ice water (200 mL) and the resulting mixture was extracted with ethyl acetate (500 mL). The organic layer was washed with brine (3×200 mL), dried (magnesium sulfate), and concentrated. The residue was diluted with dichloromethane (100 mL), the insoluble solids were removed by filtration, and the filtrate was concentrated to provide 3-oxo-3,4-dihydro-2H-benzo[b][1,4]oxazine-7-sulfonyl chloride in 11% yield as a yellow solid. Data: ¹HNMR (400 MHz, CDCl₃) δ 4.73 (s, 2H), 7.00 (m, 1H), 7.28 (d, 1H), 7.71 (d, 1H), 8.27 (s, 1H).

### Intermediate 30: Synthesis of 3-oxo-3,4-dihydro-2H-pyrido[3,2-b][1,4]oxazine-7-sulfonyl chloride

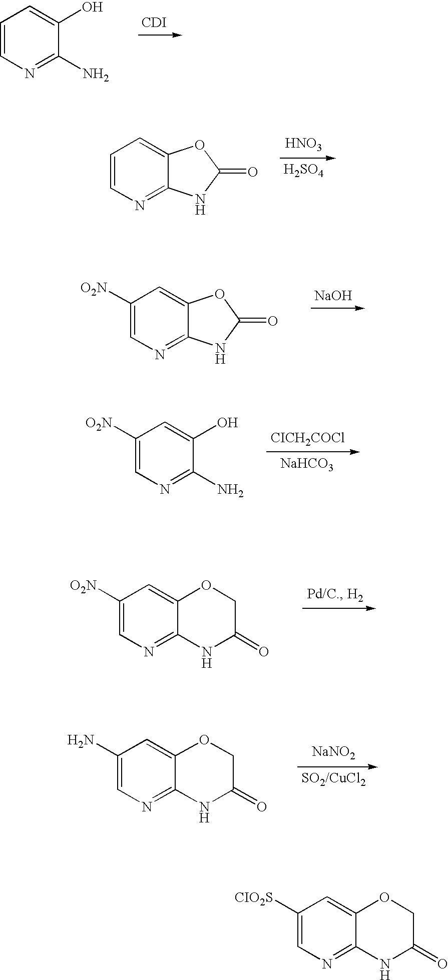

### 1. Synthesis of oxazolo[4,5-b]pyridin-2(3H)-one

Carbonyldiimidazole (600 mmol) was added in several batches to a solution of 2-aminopyridin-3-ol (400 mmol) in tetrahydrofuran (600 ml) and the reaction was heated at reflux for 1 h. The mixture was concentrated and the residue was diluted with dichloromethane (500 ml). The solution was extracted with 1.5 N sodium hydroxide (3×200 ml). The pH of the aqueous layer was adjusted to 5 with 2 N hydrochloric acid and the precipitated solids were collected by filtration to provide oxazolo[4,5-b]pyridin-2(3H)-one in 79% yield as a grey solid.

### 2. Synthesis of 6-nitrooxazolo[4,5-b]pyridin-2(3H)-one

Nitric acid (80 ml) was added to a solution of oxazolo[4,5-b]pyridin-2(3H)-one (318 mmol) in sulfuric acid (160 ml) at −5° C. The reaction mixture was allowed to warm to rt and was maintained for 60 h. The reaction mixture was diluted with ice water (200 ml) and the precipitated solids were collected by filtration, washed with water, and dried to provide 6-nitrooxazolo[4,5-b]pyridin-2(3H)-one in 39% yield as a light yellow solid.

### 3. Synthesis of 2-amino-5-nitropyridin-3-ol

A solution of sodium hydroxide (500 mmol) in water (180 ml) was added to a solution of 6-nitrooxazolo[4,5-b]pyridin-2(3H)-one (124 mmol) in ethanol (100 ml) and the reaction mixture was heated at 80° C. for 3 h. The reaction mixture was quenched with concentrated hydrochloric acid (40 mL) and the pH adjusted to 8 with 2 M sodium carbonate. The precipitated solids were collected by filtration to provide 2-amino-5-nitropyridin-3-ol in 86% yield as a yellow solid.

### 4. Synthesis of 3-oxo-3,4-dihydro-2H-pyrido[3,2-b][1,4]oxazine-7-sulfonyl chloride

The conversion of 2-amino-5-nitropyridin-3-ol to 3-oxo-3,4-dihydro-2H-pyrido[3,2-b][1,4]oxazine-7-sulfonyl chloride was achieved using the procedure to prepare intermediate 29. 3-Oxo-3,4-dihydro-2H-pyrido[3,2-b][1,4]oxazine-7-sulfonyl chloride was isolated as a light yellow solid. Data: ¹H NMR (CDCl₃) δ 8.81 (s, 1H), 8.60 (m, 1H), 7.80 (m, 1H), 4.81 (m, 2H). LC/MS (ES) m/z 247 [M+1]⁺.

### Intermediate 31: Synthesis of 4-methyl-3,4-dihydro-2H-benzo[b][1,4]oxazine-6-sulfonyl chloride

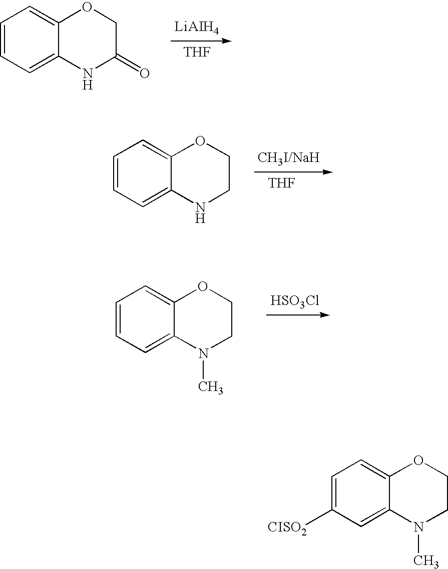

### 1. Synthesis of 3,4-dihydro-2H-benzo[b][1,4]oxazine

A solution of 2H-benzo[b][1,4]oxazin-3(4H)-one (38.2 mmol) in tetrahydrofuran (21 mL) was slowly added to a suspension of lithium aluminum hydride (94.7 mmol) in tetrahydrofuran (80 mL) and the reaction mixture was heated at reflux for 16 h. The reaction mixture was diluted with water (3.6 mL) and 15% sodium hydroxide (10.8 mL) and the insoluble solids were removed by filtration. The aqueous layer was extracted with ethyl acetate (2×100 mL) and the combined organic layers were dried (sodium sulfate) and concentrated to provide 3,4-dihydro-2H-benzo[b][1,4]oxazine in 79% yield as red oil.

### 2. Synthesis of 4-methyl-3,4-dihydro-2H-benzo[b][1,4]oxazine

Sodium hydride (57.5 mmol) was added in several batches to a solution of 3,4-dihydro-2H-benzo[b][1,4]oxazine (35.5 mmol) in tetrahydrofuran (50 mL) at 0° C. and the reaction mixture was maintained for 30 min. Iodomethane (63.4 mmol) was added dropwise and the reaction mixture was allowed to warm to rt and was maintained for 16 h. The insoluble solids were removed by filtration and the filtrate was concentrated. The residue was purified by Flash chromatography (1/100 ethyl acetate/petroleum ether) to provide 4-methyl-3,4-dihydro-2H-benzo[b][1,4]oxazine in 50% yield as yellow oil.

### 3. Synthesis of 4-methyl-3,4-dihydro-2H-benzo[b][1,4]oxazine-6-sulfonyl chloride

4-Methyl-3,4-dihydro-2H-benzo[b][1,4]oxazine (38.9 mmol) was added dropwise to sulfurochloridic acid (25 mL) and the reaction mixture was maintained for 120 min at rt. The reaction mixture was diluted with ice water and was extracted with ethyl acetate (3×200 mL). The combined organic layers were dried (sodium sulfate) and concentrated. The solid residue was washed with hexane (3×15 mL) and dried to provide 4-methyl-3,4-dihydro-2H-benzo[b][1,4]oxazine-6-sulfonyl chloride in 27% yield as a light yellow solid. Data: ¹H NMR (CDCl₃) δ 2.98 (s, 3H), 3.36 (m, 2H), 4.38 (m, 2H), 6.87 (d, 1H), 7.19 (s, 1H), 7.34 (d, 1H). LC/MS (ES) m/z 319 [M+BnNH+H]⁺.

### Intermediate 32: Synthesis of 2-oxo-2,3-dihydrobenzo[d]oxazole-6-sulfonyl chloride

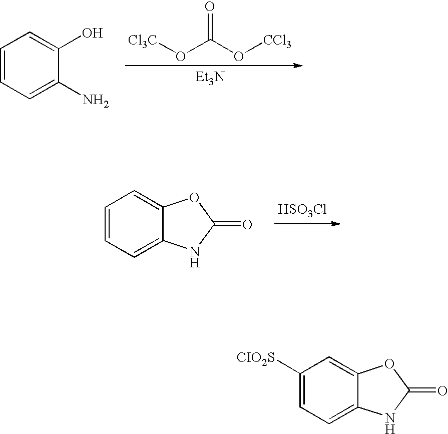

### 1. Synthesis of benzo[d]oxazol-2(3H)-one

A solution of bis(trichloromethyl) carbonate (31.5 mmol) in dichloromethane (40 mL) was added to a solution of 2-aminophenol (91.7 mmol) and triethylamine (27.0 mL) in dichloromethane (200 mL) at 5° C. The resulting solution was maintained below 10° C. for 6 h and was diluted with water (50 mL) and ethanol (20 mL). After 30 min, the reaction mixture was concentrated and resuspended in water (400 mL). The precipitated solids were collected by filtration and were was washed with hydrochloric acid (10%) and water to afford benzo[d]oxazol-2(3H)-one in 48% yield as an off-white solid.

### 2. Synthesis of 2-oxo-2,3-dihydrobenzo[d]oxazole-6-sulfonyl chloride

Sulfurochloridic acid (604 mmol) was cooled to 0° C. and benzo[d]oxazol-2(3H)-one (13.3 mmol) was added in several batches. The resulting solution was maintained at rt for 3 h and was diluted with iced water (400 mL). The resulting mixture was extracted with ethyl acetate (3×100 mL) and the combined organic layers were dried (sodium sulfate), filtered and concentrated. The residue was purified by Flash chromatography (1/10 ethyl acetate/petroleum ether) to afford 2-oxo-2,3-dihydrobenzo[d]oxazole-6-sulfonyl chloride in 26% yield as a white solid. ¹H NMR (CDCl₃) δ 8.26 (s, 1H), 8.00 (d, 1H), 7.98 (d, 1H), 7.32 (s, 1H).

### Intermediate 33: Synthesis of 3-methyl-2-oxo-2,3-dihydrobenzo[d]oxazole-6-sulfonyl chloride

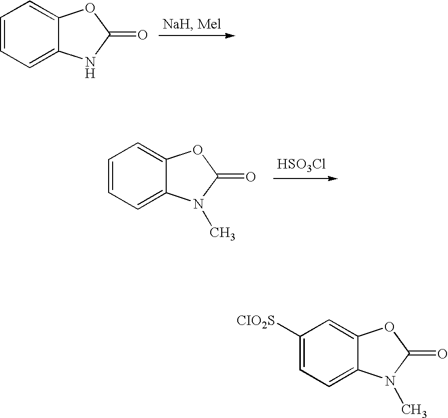

### 1. Synthesis of 3-methylbenzo[d]oxazol-2 (3 Hz-one

Sodium hydride (7.00 mmol) was added to a chilled (0° C.) solution of benzo[d]oxazol-2(3H)-one (4.81 mmol) in tetrahydrofuran (20 mL) and the reaction mixture was maintained for 30 min. Methyl iodide (7.25 mmol) was added dropwise and the reaction mixture was maintained for 6 h at rt. The reaction mixture was diluted with ethanol (10 mL) and the mixture was concentrated. The residue was diluted with water (50 mL) and was extracted with dichloromethane (3×20 mL). The combined organic layers were dried (sodium sulfate), filtered and concentrated to afford 3-methylbenzo[d]oxazol-2(3M)-one in 820% yield as a light red solid.

### 2. Synthesis of 3-methyl-2-oxo-2,3-dihydrobenzo[d]oxazole-6-sulfonyl chloride

3-Methylbenzo[d]oxazol-2(3H)-one (4.16 mmol) was added in several batches to sulfurochloridic acid (17.5 g) at 0° C. The resulting solution was allowed to warm to rt and was maintained for 3 h. The reaction mixture was slowly poured into cold (0° C.) brine (200 mL) and the resulting solution was extracted with ethyl acetate (3×40 mL). The combined organic layers were dried (sodium sulfate), filtered and concentrated to afford 3-methyl-2-oxo-2,3-dihydrobenzo[d]oxazole-6-sulfonyl chloride in 46% yield as a light brown solid. ¹H NMR (CDCl₃) δ 8.00 (d, 1H), 7.97 (s, 1H), 7.16 (d, 1H), 3.52 (s, 3H).

### Intermediate 34: Synthesis of 1-methylindoline-6-sulfonyl chloride

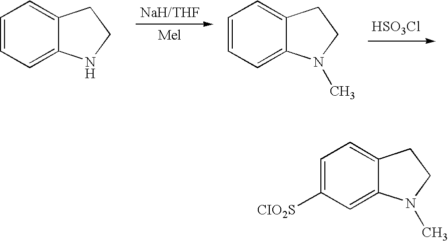

### 1. Synthesis of 1-methylindoline

Sodium hydride (375 mmol) was added in several batches to a chilled (O ° C.) solution of indoline (252 mmol) in tetrahydrofuran (400 mL). Methyl iodide (373 mmol) was then added dropwise with stirring, while maintaining the temperature of 0° C. The resulting solution was maintained at rt for 15 h, then diluted with ethanol (200 mL). The mixture was concentrated, water (400 mL) was added, and the product was extracted with dichloromethane (3×200 mL). The organics were combined, dried (sodium sulfate), filtered and concentrated to provide 1-methylindoline in 60% yield as a brown liquid.

### 2. Synthesis of 1-methylindoline-6-sulfonyl chloride

Sulfurochloridic acid (400 g) was cooled to 0° C. and 1-methylindoline (263 mmol) was added dropwise with stirring, maintaining the temperature at 0° C. The resulting solution was then warmed to rt and stirred for 20 h. The reaction mixture was added carefully then dropwise to 3 L of iced water and the resulting solution was extracted with dichloromethane (3×400 mL). The organic layers were combined, dried (sodium sulfate) and concentrated. The resulting residue was purified by Flash chromatography (1/30 ethyl acetate/petroleum ether). The collected fractions were combined and concentrated to give 1-methyl indoline-6-sulfonyl chloride in 7% yield as a brown solid. ¹H NMR (CDCl₃) δ 7.34 (d, 1H), 7.20 (d, 1H), 6.95 (s, 1H), 3.52 (t, 2H), 3.08 (t, 2H), 2.86 (s, 3H).

### 3. Synthesis of 1-ethylindoline-6-sulfonyl chloride

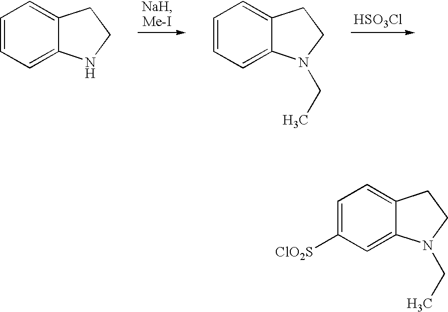

1-Ethylindoline-6-sulfonyl chloride was obtained as a yellow solid using this procedure. Data: ¹H NMR (CDCl₃) δ 7.28 (d, 1H), 7.18 (d, 1H), 7.11 (s, 1H), 3.39 (q, 2H), 3.52 (t, 2H), 3.06 (t, 2H), 1.23 (t, 3H).

### Intermediate 35: Synthesis of 1-methylindoline-5-sulfonyl chloride

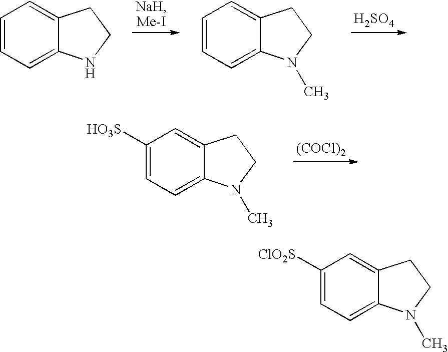

### 1. Synthesis of 1-methylindoline

Sodium hydride (150 mmol) was added to a chilled (0° C.) solution of indoline (101 mmol) in tetrahydrofuran (200 mL). The resulting solution was then stirred at rt for 30 minutes. Iodomethane (141 mmol) was then added dropwise and the resulting solution was maintained at rt for an additional 16 h. The reaction mixture was concentrated and the residue was diluted with water (200 mL) and extracted with dichloromethane (2×200 mL). The combined organic layers were dried (sodium sulfate) and concentrated to give 1-methylindoline in 34% yield as yellow liquid.

### 2. Synthesis of 1-methylindoline-5-sulfonic acid

Sulfuric acid (38.1 mmol) was added to a solution of 1-methylindoline (37.5 mmol) in ether (20 mL) and the reaction mixture was maintained at rt for 30 min. The reaction mixture was then heated at 170° C. under vacuum for 3 h. The reaction was diluted with methanol (100 mL) and the precipitated solids were collected by filtration to give 1-methylindoline-5-sulfonic acid in 16% yield as a colorless solid.

### 3. Synthesis of 1-methylindoline-5-sulfonyl chloride

Oxalyl chloride (33.1 mmol) was added to a solution of 1-methylindoline-5-sulfonic acid (6.56 mmol) in dichloromethane (20 mL) and N,N-dimethylformamide (0.5 mL) and the reaction mixture was maintained at rt for 2 h. The reaction was washed with water (100 mL), dried (sodium sulfate), and concentrated to give 1-methylindoline-5-sulfonyl chloride in 62% yield as a yellow solid. ¹H NMR (CDCl₃) δ 7.74 (d, 1H), 7.56 (s, 1H), 6.33 (d, 1H), 3.63 (t, 2H), 3.08 (t, 2H), 2.90 (s, 3H).

### Intermediate 36: Synthesis of benzo[d]isoxazole-5-sulfonyl chloride

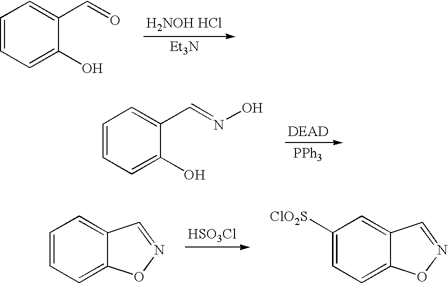

### 1. Synthesis of (L-2-hydroxybenzaldehyde oxime

Triethylamine (190 mmol) was added slowly to a solution of 2-hydroxybenzaldehyde (164 mmol) and hydroxylamine hydrochloride (197 mmol) in ethanol (200 mL) and the reaction mixture was heated at 95° C. for 5 h. The reaction mixture was concentrated and the residue was extracted with ethyl acetate (2×150 mL) and water (100 mL). The combined organic layers were washed with water (3×150 mL), dried (magnesium sulfate), and concentrated. The residue was purified by Flash chromatography (11/00 ethyl acetate/petroleum ether) to provide (A)-2-hydroxybenzaldehyde oxime in 43% yield as a white solid.

### 2. Synthesis of benzo[d]isoxazole

A solution of DEAD (23.0 mmol) in tetrahydrofuran (150 mL) was added over a period of 4 h to a solution of (E)-2-hydroxybenzaldehyde oxime (21.9 mmol) and triphenylphosphine (23.0 mmol) in tetrahydrofuran (300 mL) at 0° C. The reaction mixture was maintained at 0° C. for an additional 60 min and was concentrated. The residue was purified by Flash chromatography (1/100 ethyl acetate/petroleum ether) to provide benzo[d]isoxazole in 66% yield as yellow oil.

### 3. Synthesis of benzo[d]isoxazole-5-sulfonyl chloride

Benzo[d]isoxazole (4.20 mmol) was added dropwise over 20 min to sulfurochloridic acid (2.8 mL) at 0° C. and the reaction mixture was heated at 100° C. for 27 h. The reaction mixture was diluted by dichloromethane and cautiously poured into ice water (50 mL). The aqueous layer was extracted with dichloromethane (2×50 mL). The combined organic layers were washed with water (2×50 mL), dried (magnesium sulfate), and concentrated to provide benzo[d]isoxazole-5-sulfonyl chloride in 48% yield as a red solid. Data: ¹H NMR (CDCl₃) δ 8.93 (s, 1H), 8.54 (s, 1H), 8.26 (d, 1H), 7.87 (d, 1H). LC/MS (ES) m/z 287 [M+BnNH−H]⁻.

### Intermediate 37: Synthesis of 2,3-dihydrobenzofuran-4-sulfonyl chloride

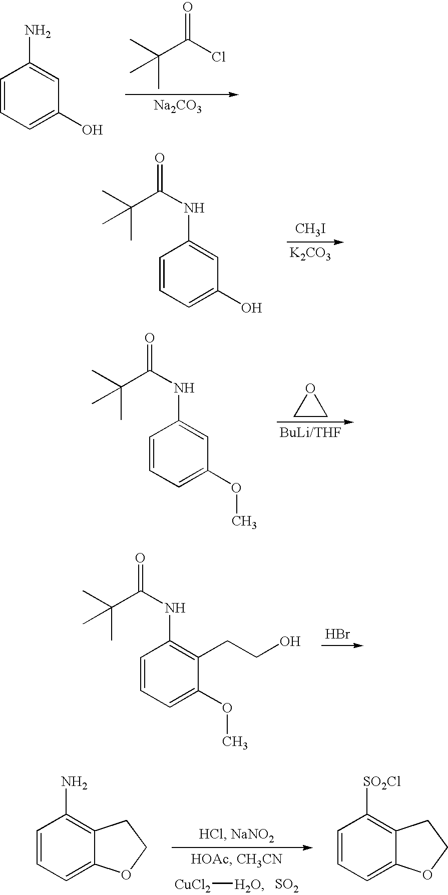

### 1. Synthesis of N-(3-hydroxyphenyl)pivalamide

Pivaloyl chloride (38.3 mmol) was added dropwise to a biphasic mixture of 3-aminophenol (36.5 mmol) and sodium carbonate (86.8 mmol) in ethyl acetate (125 mL) and water (150 mL) at 0° C. The resulting solution was stirred vigorously for 1 h and the layers were separated. The organic phase was washed with 1 N hydrochloric acid, water, and brine, was dried (sodium sulfate), and was concentrated to provide N-(3-hydroxyphenyl)pivalamide in 90% yield as a gray solid.

### 2. Synthesis of N-(3-methoxyphenyl)pivalamide

Methyl iodide (277 mmol) was added to a suspension of N-(3-hydroxyphenyl)pivalamide (69.4 mmol) and potassium carbonate (207 mmol) in acetone (500 mL) and the reaction mixture was heated at reflux for 3 h. The insoluble solids were removed by filtration and the filtrate was concentrated. The residue was extracted with hexane (3×300 mL) and the combined extracts were concentrated to provide N-(3-methoxyphenyl)pivalamide in 91% yield as a white solid.

### 3. Synthesis of N-(2-(2-hydroxyethyl)-3-methoxyphenyl)pivalamide

A solution of n-butyllithium in hexane (60 mL) was added dropwise to a solution of N-(3-methoxyphenyl)pivalamide (57.0 mmol) in tetrahydrofuran (200 mL) at 0° C. and was maintained for 2 h. Oxirane (86 mmol) was added dropwise and the reaction mixture was maintained for 1 h at 0° C. and for an additional 2 h at rt. The reaction mixture was concentrated and the residue was diluted with water (100 mL) and extracted with ethyl acetate (3×75 mL). The combined organic layers were washed with saturated aqueous sodium carbonate, dried (sodium sulfate), and concentrated. The final product was purified by recrystallization (dichloromethane/cyclohexane) to provide N42-(2-hydroxyethyl)-3-methoxyphenyl)pivalamide in 53% yield as a white solid.

### 4. Synthesis of 2,3-dihydrobenzofuran-4-amine

Concentrated hydrobromic acid (100 mL) was added to N-(2-(2-hydroxyethyl)-3-methoxyphenyl)pivalamide (41.8 mmol) and the reaction mixture was heated at 100° C. for 16 h. The pH of the solution was adjusted to 9 with solid sodium hydroxide and the solution was extracted with ethyl acetate (3×100 mL). The combined organic layers were was washed with water (50 mL), dried (sodium sulfate), and concentrated to provide 2,3-dihydrobenzofuran-4-amine in 40% yield as yellow oil.

### 5. Synthesis of 2,3-dihydrobenzofuran-4-sulfonyl chloride

Hydrochloric acid (9.0 g) was added dropwise to a solution of 2,3-dihydrobenzofuran-4-amine (16.3 mmol) and acetic acid (9.0 g) in acetonitrile (200 mL) at 0° C. A solution of sodium nitrite (22.0 mmol) in water (2 mL) was subsequently added and the mixture was maintained for 30 min at 0° C. Sulfur dioxide gas was passed through the reaction mixture for 2 h whereupon a solution of copper(II) chloride dihydrate (20.0 mmol) in water (3 mL) was added. Sulfur dioxide gas was passed through the reaction mixture for an additional 2 h. The reaction mixture was allowed to warm to rt and was maintained for 16 h. The reaction mixture was diluted with ice water (200 mL) and the resulting mixture was extracted with ethyl acetate (300 mL). The organic layer was washed with water (200 mL), dried (sodium sulfate), and concentrated. The residue was purified by Flash chromatography (1/70 ethyl acetate/petroleum ether) to provide 2,3-dihydrobenzofuran-4-sulfonyl chloride in 40% yield as a yellow solid. Data: ¹H NMR (CDCl₃) δ 7.40 (d, 1H), 7.30 (d, 1H), 7.10 (d, 1H), 4.70 (m, 2H), 3.60 (m, 2H). LC/MS (ES) In/z 283 [M+C5H11N2-Cl+H]⁺.

### Intermediate 38: Synthesis of 2,3-dihydrobenzofuran-6-sulfonyl chloride

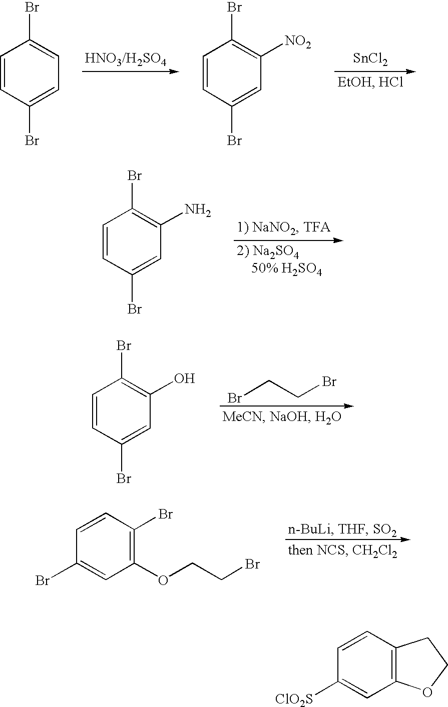

### 1. Synthesis of 1,4-dibromo-2-nitrobenzene

A mixture of 68% nitric acid/98% sulfuric acid (32/64 mL) was added dropwise to a solution of 1,4-dibromobenzene (100 mmol) in 98% sulfuric acid (40 mL) and the reaction mixture was heated at 50° C. for 30 min. The reaction mixture was allowed to cool to rt, was diluted with ice water (200 mL), and was extracted with dichloromethane (3×200 mL). The combined organic layers were washed with water (2×100 mL) and 10% potassium hydroxide (3×100 mL), dried (magnesium sulfate), and concentrated. The residue was purified by Flash chromatography (petroleum ether) to provide 1,4-dibromo-2-nitrobenzene in 68% yield as a light green-yellow solid.

### 2. Synthesis of 2,5-dibromobenzenamine

A solution of 1,4-dibromo-2-nitrobenzene (64.1 mmol) in ethanol (40 mL) was added to a solution of tin(II) chloride hydrate (192 mmol) in concentrated hydrochloric acid (40 mL) and the reaction mixture was heated at reflux for 1 h. The reaction mixture was allowed to cool to rt and was maintained for an additional 2 h. The pH of the aqueous layer was adjusted to 8-9 with 50% sodium hydroxide and the resulting solution was extracted with ethyl acetate (3×200 mL), dried (sodium sulfate), and concentrated to provide 2,5-dibromobenzenamine in 97% yield as a yellow solid.

### 3. Synthesis of 2,5-dibromophenol

Sodium nitrite (65.2 mmol) was added in several portions to a solution of 2,5-dibromobenzenamine (55.8 mmol) in trifluoroacetic acid (80 mL) at 0° C. The resulting solution was added to a boiling solution of sodium sulfate (10 g) in 50% sulfuric acid (120 mL) and the reaction mixture was maintained at reflux for 1 h. Then the product was steam-distilled and the distillate was extracted with dichloromethane (2×200 mL). The combined organic layers were dried (sodium sulfate) and concentrated to provide 2,5-dibromophenol in 41% yield as a yellow solid.

### 4. Synthesis of 1,4-dibromo-2-(2-bromoethoxy)benzene

1,2-Dibromoethane (23.5 mmol) was added to a solution of 2,5-dibromophenol (23.8 mmol) in acetonitrile (20 mL) and 1.15 M sodium hydroxide in water (20 mL) and the reaction mixture was heated at reflux for 16 h. The reaction mixture was concentrated to 1×3 volume and was extracted with ethyl acetate (3×50 mL). The combined organic layers were dried (sodium sulfate) and concentrated. The residue was purified by Flash chromatography (1/10 ethyl acetate/hexane) to provide 1,4-dibromo-2-(2-bromoethoxy)benzene in 49% yield as a white solid.

### 5. Synthesis of 2,3-dihydrobenzofuran-6-sulfonyl chloride

n-Butyllithium (13.6 mmol) was added dropwise to a solution of 1,3-dibromo-2-(2-bromnoethoxy)benzene (12.8 mmol) in tetrahydrofuran (100 mL) at −100° C. and the reaction mixture was maintained for 60 min. n-Butyllithium (13.6 mmol) was added dropwise and the reaction mixture was maintained at −100° C. for an additional 30 min. Sulfur dioxide (25.8 mmol) was added and the reaction mixture was warmed to −40° C. and was maintained for an additional 60 min. The reaction mixture was concentrated and the residue was suspended in dichloromethane (100 mL) at 0° C. N-Chlorosuccinamide (14.5 mmol) was added in several batches and the reaction mixture was maintained for 60 min at 0° C. The reaction mixture was diluted with dichloromethane (100 mL) and was washed with (2 M) sodium hydrogen sulfate (2×150 mL) and brine (3×100 mL), dried (sodium sulfate), and concentrated. The residue was purified by Flash chromatography (1/50 ethyl acetate/petroleum ether) to provide 2,3-dihydrobenzofuran-6-sulfonyl chloride in 41% yield as a white solid. Data: ¹H NMR: (DMSO-d6) δ 7.55 (t, 1H), 7.41 (d, 1H), 7.35 (d, 1H), 3.44 (t, 2H), 4.73 (t, 2H). LC/MS (ES) m/z 283 [M+C₅H₁₂N₂-hydrochloric acid]⁺.

### Intermediate 39: Synthesis of 2,3-dihydrobenzofuran-7-sulfonyl chloride

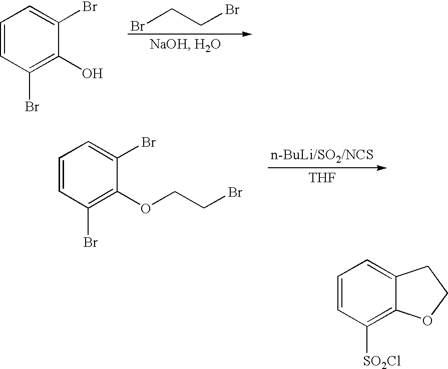

### 1. Synthesis of 1,3-dibromo-2-(2-bromoethoxy)benzene

1,2-Dibromoethane (58 mmol) was added dropwise to a solution of 2,6-dibromophenol (57.5 mmol) and sodium hydroxide (62.5 mmol) in water (45 mL) and the reaction mixture was heated at reflux for 17 h. The reaction mixture was allowed to cool to rt and was extracted with diethyl ether (2×100 mL). The combined organic layers were washed with 1 M sodium hydroxide (100 mL) and brine (100 mL), dried (sodium sulfate), and concentrated. The residue was purified by Flash chromatography (1/1000 ethyl acetate/petroleum) to provide 1,3-dibromo-2-(2-bromoethoxy)benzene in 69% yield as a colorless liquid.

### 2. Synthesis of 2,3-dihydrobenzofuran-7-sulfonyl chloride

n-Butyllithium (23 mmol) was added dropwise to a solution of 1,3-dibromo-2-(2-bromoethoxy)benzene (21.8 mmol) in tetrahydrofuran (100 mL) at −100° C. and the reaction mixture was maintained for 30 min. n-Butyllithium (23 mmol) was added dropwise and the reaction mixture was maintained at −100° C. for an additional 60 min. Sulfur dioxide (43.8 mmol) was added and the reaction mixture was maintained for 2 h between −100 and −85° C. The reaction mixture was diluted with hexane (100 mL) and the precipitated solids were collected by filtration. The solid was suspended in dichloromethane (100 mL) at 0° C. and N-chlorosuccinamide (24.6 mmol) was added in several batches. The reaction mixture was maintained for 60 min at 0° C. and was diluted with dichloromethane (100 mL). The reaction mixture was washed with (2 M) sodium hydrogen sulfate (2×150 mL) and brine (3×100 mL), was dried (sodium sulfate), and was concentrated. The residue was purified by Flash chromatography (1/50 ethyl acetate/petroleum ether) to provide 2,3-dihydrobenzofuran-7-sulfonyl chloride in 51% yield as a light yellow solid. Data: ¹HNMR: (CDCl₃) δ 3.35 (t, 2H), 4.92 (t, 2H), 6.96 (t, 1H), 7.54 (s, 1H), 7.64 (d, 1H). LC/MS (ES) m/z 283 [C13H18N₂O3S+H]⁺.

### Intermediate 40: Synthesis of 1-methyl-1H-indazole-5-sulfonyl chloride

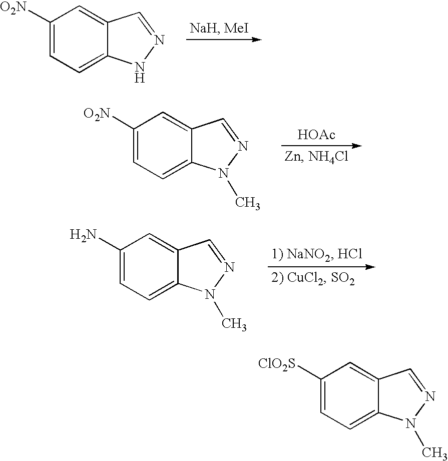

### 1. Synthesis of 1-methyl-5-nitro-1H-indazole

Sodium hydride (55.0 mmol) was added to a solution of 5-nitro-1H-indazole (18.40 mmol) in N,N-dimethylformamide (50 mL) and the mixture was maintained for 60 min at 0° C. To the mixture was added Methyl iodide (22.12 mmol) was added and the reaction mixture was allowed to warm to rt and was maintained for 18 h. The reaction mixture was quenched with water (60 mL), filtered through Celite, and the filtrate was concentrated to provide 1-methyl-5-nitro-1H-indazole in 83% yield as a yellow solid.

### 2. Synthesis of 1-methyl-1H-indazol-5-amine

Zinc powder (194 mmol), ammonium chloride (388 mmol), and acetic acid (33.3 mmol) were added, successively, to a solution of 1-methyl-5-nitro-1H-indazole (19.1 mmol) in ethanol (50 mL), water (20 mL), and ethyl acetate (5 mL) and the resulting suspension was maintained at rt for 1 h. The insoluble solids were removed by filtration and the filtrate was concentrated. The residue was purified by Flash chromatography (5/1 petroleum ether/ethyl acetate) to provide 1-methyl-1H-indazol-5-amine in 18% yield as a brown solid.

### 3. Synthesis of 1-methyl-1H-indazole-5-sulfonyl chloride

A solution of sodium nitrite (24.2 mmol) in water (2 mL) was added to a solution of 1-methyl-1H-indazol-5-amine (20.4 mmol) in concentrated hydrochloric acid (10 mL) and the mixture was maintained for 60 min at 0° C. in a second reaction vessel, sulfur dioxide gas was passed through a mixture of acetic acid (10 mL) and acetonitrile (10 mL) until the saturation point was reached. Solid copper(II) chloride dihydrate (21.8 mmol) was added to the sulfur dioxide solution and the solution of the indazole diazo salt was subsequently added over a period of 30 min. The reaction mixture was allowed to warm to rt and was maintained for 24 h. The reaction mixture was diluted with ice water (80 mL) and the insoluble solids were removed by filtration. The filtrate was extracted with ethyl acetate (2×50 mL) and the combined organic layers were dried (magnesium sulfate), and concentrated to provide 1-methyl-1H-indazole-5-sulfonyl chloride in 53% yield as a yellow solid. Data: LC/MS (ES) m/z 300 [M+BnNH+1]⁺.

### Intermediate 41: Synthesis of benzofuran-5-sulfonyl chloride

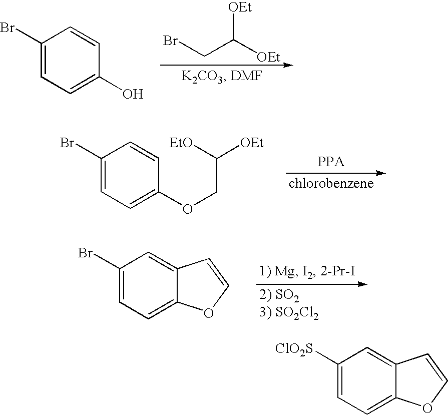

### 1. Synthesis of 1-bromo-4-(2,2-diethoxyethoxy)benzene

2-Bromo-1,1-diethoxyethane (63.9 mmol) was added to a suspension of 4-bromophenol (57.8 mmol) and potassium carbonate (87.0 mmol) in N,N-dimethylformamide (80 mL) and the reaction mixture was heated at 100° C. for 16 h. The resulting solution was diluted with water (200 mL) and was extracted with ethyl acetate (3×150 mL). The combined organic layers were washed with brine (5×100 mL), dried (sodium sulfate), and concentrated to provide 1-bromo-4-(2,2-diethoxyethoxy)benzene as yellow oil.

### 2. Synthesis of 5-bromobenzofuran

Phosphoric acid (40 g) was added to a solution of 1-bromo-4-(2,2-diethoxyethoxy)benzene (51.9 mmol) in chlorobenzene (80 mL) and the reaction mixture was heated at reflux for 16 h. The reaction mixture was allowed to cool to rt and the chlorobenzene layer was decanted. The residue was washed with toluene (2×30 mL) and the combined organic layers were concentrated. The residue was purified by Flash chromatography (hexane) to provide 5-bromobenzofuran in 50% yield as colorless oil.

### 3. Synthesis of benzofuran-5-sulfonyl chloride

Isopropyl iodide (15.0 mmol) was added dropwise to a suspension of iodine (0.12 mmol), magnesium (30.0 mmol) in tetrahydrofuran (25 mL). After 15 mill, a solution of 5-bromobenzofuran (15.2 mmol) in tetrahydrofuran (25 mL) was added dropwise and the reaction mixture was heated at reflux for 1 h. The mixture was cooled to −30° C. and sulfonyl chloride was bubbled through the reaction mixture for 10 min. The mixture was maintained for 30 min whereupon sulfuryl chloride (15.1 mmol) was added dropwise while cooling to −30 to −40° C. The resulting solution was maintained for an additional 10 min and was allowed to warm to rt. The insoluble solids were removed by filtration and the filtrate was concentrated. The residue was diluted with dichloromethane (150 mL), washed with brine (3×100 mL), dried (sodium sulfate), and concentrated. The residue was purified by Flash chromatography (100/1 to 50/1 petroleum ether/ethyl acetate) to provide benzofuran-5-sulfonyl chloride in 15% yield as a white solid. Data: ¹H NMR (CDCl₃) δ 8.37 (s, 1H), 8.00 (d, 1H), 7.84 (s, 1H), 7.44 (d, 1H), 6.97 (s, 1H). LC/MS (ES) In/z 286 [M+BnH−1]⁺.

### Intermediate 42: Synthesis of benzothiazole-5-sulfonyl chloride

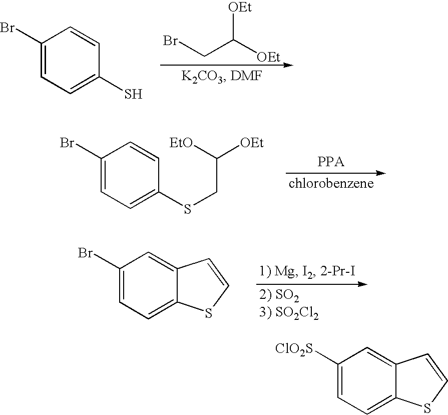

Benzothiazole-5-sulfonyl chloride was prepared from 4-bromothiophenol using the method to prepare intermediate 41.

### Intermediate 43: Synthesis of 2,3-dimethoxybenzene-1-sulfonyl chloride

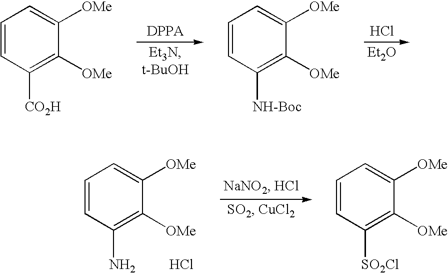

### 1. Synthesis of tert-butyl 2,3-dimethoxyphenylcarbamate

Triethylamine (17.6 mL) and tert-butanol (100 mL) were added to a solution of 2,3-dimethoxybenzoic acid (120 mmol) and DPPA (27.2 mL) in 1,4-dioxane (334 mL) and the reaction mixture was heated at reflux for 16 h. The mixture was concentrated and the residue was diluted with ethyl acetate (200 mL) and was washed with. The resulting mixture was washed with saturated sodium carbonate (3×600 mL) and brine (3×600 mL), dried (sodium sulfate), and concentrated. The residue was purified by Flash chromatography (100/1 to 60/1 petroleum ether/ethyl acetate) to provide tert-butyl 2,3-dimethoxyphenylcarbamate in 61% yield as light yellow oil.

### 2. Synthesis of 2,3-dimethoxybenzenamine hydrochloride

Hydrochloric acid was bubbled through a solution of tert-butyl 2,3-dimethoxyphenylcarbamate (26.5 mmol) in ether (150 mL) for 15 min and the resulting solution was maintained for 4 h at rt. The precipitated solids were collected by filtration, washed with ether, and dried to provide 2,3-dimethoxybenzenamine hydrochloride in 87% yield as a white solid.

### 3. Synthesis of 2,3-dimethoxybenzene-1-sulfonyl chloride

Hydrochloric acid (13 mL) was added to a solution of 2,3-dimethoxybenzenamine hydrochloride (23.2 mmol) in acetic acid (12 mL) and acetonitrile (250 mL) at 0° C. A solution of sodium nitrite (27.8 mmol) in water (5 mL) was subsequently added and the mixture was maintained for 30 min at 0° C. Sulfur dioxide gas was bubbled through the solution for 2 h while the temperature was maintained at 0° C. Solid copper(II) chloride dihydrate (28.0 mmol) was added in portions and sulfur dioxide gas was bubbled through the solution for an additional 60 min. The reaction mixture was allowed to warm to rt and was maintained for 16 h. The reaction mixture was diluted with ice water (400 mL) and the resulting mixture was extracted with dichloromethane (3×300 mL). The combined organic layers were washed with brine (3×200 mL), dried (sodium sulfate), and concentrated. The residue was purified by Flash chromatography (40/1 petroleum ether/ethyl acetate) to provide 2,3-dimethoxybenzene-1-sulfonyl chloride in 81% yield as a white solid. Data: ¹H NMR (CDCl₃) 7.53 (dd, 1H), 7.29 (dd, 1H), 7.23 (t, 1H), 4.10 (s, 3H), 3.96 (s, 3H). LC/MS (ES) m/z 301 [M+C5H11N2-Cl]⁺.

### Intermediate 44: Synthesis of 2,3-dihydrobenzo[b][1,4]dioxine-5-sulfonyl chloride

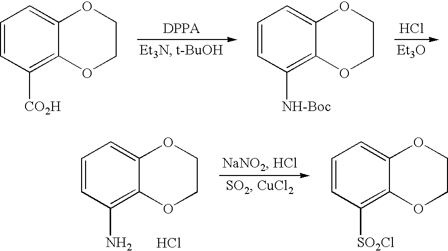

2,3-Dihydrobenzo[b][1,4]dioxine-5-sulfonyl chloride was prepared from 2,3-dihydrobenzo[b][1,4]dioxine-5-carboxylic acid using the procedure to prepare intermediate 43. Data: ¹H NMR (400 MHz, CDCl₃) δ 7.53 (m, 1H), 7.24 (m, 1H), 6.98 (m, 1H), 4.52 (m, 2H), 4.40 (m, 2H). LC/MS (ES) m/z 299 [M+C5H11N2-Cl+H]⁺.

### Intermediate 45: Synthesis of 1-ethylindoline-5-sulfonyl chloride

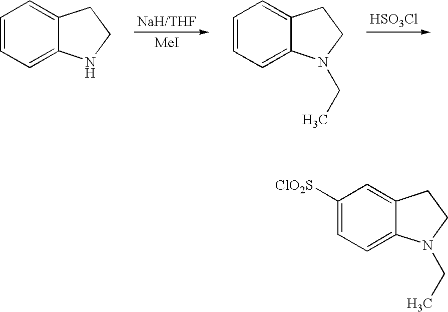

### 1. Synthesis of 1-ethylindoline

Sodium hydride (10 g) was added to a chilled (0° C.) solution of indoline (252 mmol) in tetrahydrofuran (300 mL). The resulting solution was then stirred at rt for 30 minutes. Iodoethane (323 mmol) was then added dropwise and the resulting solution was maintained at rt for an additional 3 h. The reaction mixture was diluted with water (100 mL) and was extracted with dichloromethane (3×500 mL), and the combined organic layers were concentrated. The residue was purified by Flash chromatography (100/1 ethyl acetate/petroleum ether) to give 1-ethylindoline in 78% yield as yellow oil.

### 2. Synthesis of 1-ethylindoline-5-sulfonyl chloride

1-Ethylindoline (102 mmol) was added at 0° C. to sulfurochloridic acid (60 g) and the reaction mixture was heated at 50° C. for 16 h. The reaction was diluted with ice water (300 mL) and was extracted with dichloromethane (3×600 mL). The combined organic layers were dried (magnesium sulfate) and concentrated. The residue was purified by Flash chromatography (1/100 ethyl acetate/petroleum ether) to give 1-ethylindoline-5-sulfonyl chloride in 6% yield as a yellow solid. ¹H NMR (CDCl₃) δ 7.28 (d, 1H), 7.18 (d, 1H), 7.11 (s, 1H), 3.39 (q, 2H), 3.52 (t, 2H), 3.06 (t, 2H), 1.23 (t, 3H).

Additional sulfonyl chloride starting materials can be made as described in U.S. patent application Ser. Nos. 11/676,203, 12/033,797, or 12/124,906, such synthesis is herein incorporated by reference in its entirety.

### II. Azaindole Preparations

Intermediate 46. Synthesis of 3-(hexahydropyrrolo[1,2-a]pyrazin-2(1H)-yl)-1H-pyrrolo[3,2-b]pyridine

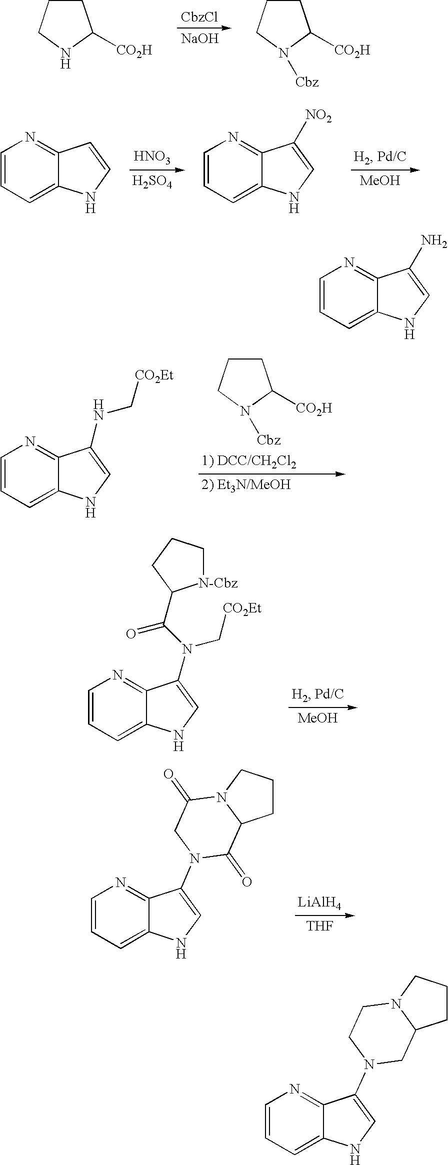

### 1. Synthesis of 1-(benzyloxycarbonyl)pyrrolidine-2-carboxylic acid

Benzyl chloroformate (174 mmol) was added dropwise to a solution of pyrrolidine-2-carboxylic acid (174 mmol) in 2 M sodium hydroxide (88 mL) at 0° C. Additional 2 M sodium hydroxide (88 mL) was added and the reaction mixture was allowed to warm to rt and was maintained for 3 h. The resulting solution was extracted with ether (50 mL) and the pH of the aqueous layers was adjusted to 4-5 with 2 M hydrochloric acid. The aqueous layer was extracted with ethyl acetate (2×100 mL) and the combined organic layers were dried (sodium sulfate) and concentrated to provide 1-(benzyloxycarbonyl)pyrrolidine-2-carboxylic acid in 88% yield as a colorless liquid.

### 2. Synthesis of 3-nitro-1H-pyrrolo[3,2-b]pyridine

Nitric acid (286 mmol) is added to a cold (0° C.) solution of 1H-pyrrolo[3,2-b]pyridine (254 mmol) in sulfuric acid (120 mL) and the reaction mixture is maintained for 2 h at 0° C. The reaction mixture is diluted with ice water (300 mL) and the pH is adjusted to 7-8 with 2 M sodium hydroxide. The precipitated solids are collected by filtration and dried to provide 3-nitro-1H-pyrrolo[3,2-b]pyridine in 89% yield as a yellow solid.

### 3. Synthesis of tert-butyl (2R)-2-methyl-4-(1H-pyrrolo[3,2-b]pyridin-3-yl)piperazine-1-carboxylate

A suspension of 3-nitro-1H-pyrrolo[3,2-b]pyridine (227 mmol) and 10% palladium on carbon (10 g) in methanol (500 mL) is maintained under an atmosphere of hydrogen gas for 5 h at rt. The catalyst is removed by filtration and the filtrate is concentrated to provide 3-amino-1H-pyrrolo[3,2-b]pyridine in 46% yield as a brown solid.

### 4. Synthesis of ethyl 2-(1H-pyrrolo[3,2-b]pyridin-3-ylamino)acetate

Ethyl 2-chloroacetate (143 mmol) was added to a solution of 1H-pyrrolo[3,2-b]pyridin-3-amine (150 mmol) in methanol (500 mL) and the reaction mixture was heated at reflux for 8 h. The reaction mixture was concentrated and the residue was purified by Flash chromatography (50/1 dichloromethane/methanol) to provide ethyl 2-(1H-pyrrolo[3,2-b]pyridin-3-ylamino)acetate in 15% yield as a red solid.

### 5. Synthesis of benzyl 2-((2-ethoxy-2-oxoethyl)(1H-pyrrolo[3,2-b]pyridin-3-yl)carbamoyl)pyrrolidine-1-carboxylate

Dicyclohexylcarbodiimide (52.4 mmol) was added to a solution of ethyl 2-(1H-pyrrolo[3,2-b]pyridin-3-ylamino)acetate (27.4 mmol) and 1-(benzyloxycarbonyl)pyrrolidine-2-carboxylic acid (108 mmol) in dichloromethane (600 mL) and the reaction mixture was maintained at rt for 48 h. The precipitated solids were removed by filtration and the filtrate was concentrated. The residue was purified by Flash chromatography (1/2 ethyl acetate/petroleum ether) to provide benzyl 2-(3-(1-(benzyloxycarbonyl)-N-(2-ethoxy-2-oxoethyl)pyrrolidine-2-carboxamido)-1H-pyrrolo[3,2-b]pyridine-1-carbonyl)pyrrolidine-1-carboxylate in 20% yield as a yellow solid.

Triethylamine (8.37 mmol) was added to a solution of benzyl 2-(3-(1-(benzyloxycarbonyl)-N-(2-ethoxy-2-oxoethyl)piperidine-2-carboxamido)-1H-pyrrolo[3,2-b]pyridine-1-carbonyl)piperidine-1-carboxylate (5.58 mmol) in methanol (100 mL) was maintained at rt for 4 h. The reaction mixture was concentrated and the residue was dissolved in dichloromethane (300 mL). The organic layer was washed with water (2×100 mL), dried (sodium sulfate), and concentrated to provide benzyl 2-((2-ethoxy-2-oxoethyl)(1H-pyrrolo[3,2-b]pyridin-3-yl)carbamoyl)pyrrolidine-1-carboxylate in 96% yield as a yellow solid.

### 6. Synthesis of 2-(1H-pyrrolo[3,2-b]pyridin-3-yl)-hexahydropyrrolo[1,2-a]pyrazine-1,4-dione

A suspension of benzyl 2-((2-ethoxy-2-oxoethyl)(1H-pyrrolo[3,2-b]pyridin-3-yl)carbamoyl)pyrrolidine-1-carboxylate (5.33 mmol) and 10% palladium on carbon (1.0 g) in methanol (200 mL) is maintained under an atmosphere of hydrogen gas for 16 h at 60° C. The catalyst is removed by filtration and the filtrate is concentrated. The residue was purified by Flash chromatography (10/90 methanol/dichloromethane) to provide 2-(1H-pyrrolo[3,2-b]pyridin-3-yl)-hexahydropyrrolo[1,2-a]pyrazine-1,4-dione in 83% yield as a yellow solid.

### 7. Synthesis of 3-(hexahydropyrrolo[1,2-a]pyrazin-2(1H-yl)-1H-pyrrolo[3,2-b]pyridine

Lithium aluminum hydride (115.6 mmol) was added to a solution of 2-(1H-pyrrolo[3,2-b]pyridin-3-yl)-hexahydropyrrolo[1,2-a]pyrazine-1,4-dione (2.59 mmol) in tetrahydrofuran (100 mL) at 0° C. The reaction mixture was heated at reflux for 2 h then was allowed to cool to rt and was quenched, successively, with water (0.6 mL), 15% sodium hydroxide (0.6 mL), and water (1.8 mL). The insoluble solids were removed by filtration and the filtrate was concentrated to provide 3-(hexahydropyrrolo[1,2-a]pyrazin-2(1-yl)-1H-pyrrolo[3,2-b]pyridine in 80% yield as a yellow solid. Data: ¹H NMR (CDCl₃) δ 8.46 (s, 1H), 7.74 (d, 1H), 7.61 (d, 1H), 7.13 (d, 1H), 6.91 (d, 1H), 4.16 (d, 1H), 3.98 (d, 1H), 3.77 (s, 1H), 3.20 (d, 2H), 2.91 (s, 1H), 2.68 (s, 1H), 2.53 (t, 2H), 2.30 (s, 1H), 1.90 (t, 3H). LC/MS (ES) m/z 243 [M+H]⁺.

### Intermediate 47. Synthesis of 2-(1H-pyrrolo[3,2-b]pyridin-3-yl)-octahydro-1H-pyrido[1,2-a]pyrazine

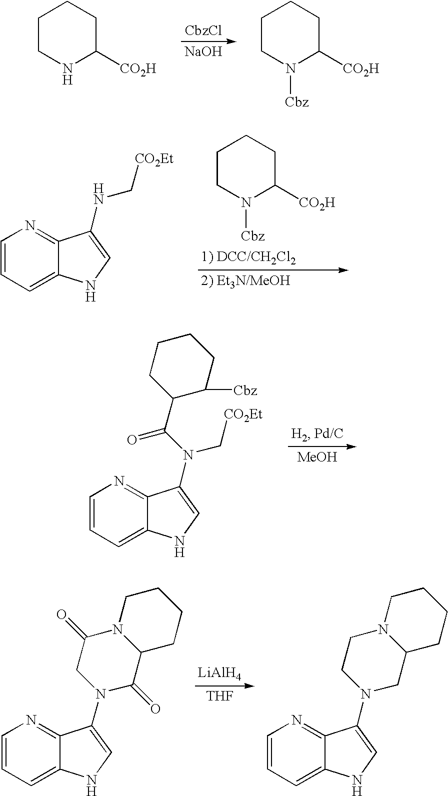

### 1. Synthesis of 1-(benzyloxycarbonyl)piperidine-2-carboxylic acid

Benzyl chloroformate (78.5 mmol) was added dropwise to a solution of piperidine-2-carboxylic acid (77.5 mmol) in 2 M sodium hydroxide (39 mL) at 0° C. Additional 2 M sodium hydroxide (39 mL) was added and the reaction mixture was allowed to warm to rt and was maintained for 2 h. The resulting solution was extracted with ether (100 mL) and the pH of the aqueous layers was adjusted to 4-5 with 6 M hydrochloric acid. The aqueous layer was extracted with ethyl acetate (2×100 mL) and the combined organic layers were dried (sodium sulfate) and concentrated to provide 1-(benzyloxycarbonyl)piperidine-2-carboxylic acid in 78% yield as a yellow oil.

### 2. Synthesis of benzyl 2-((2-ethoxy-2-oxoethyl)(1H-pyrrolo[3,2-b]pyridin-3-yl)carbamoyl)piperidine-1-carboxylate

Dicyclohexylcarbodiimide (43.8 mmol) was added to a solution of ethyl 2-(1H-pyrrolo[3,2-b]pyridin-3-ylamino)acetate (21.9 mmol) and 1-(benzyloxycarbonyl)piperidine-2-carboxylic acid (87.7 mmol) in dichloromethane (250 mL) and the reaction mixture was maintained at rt for 16 h. The precipitated solids were removed by filtration and the filtrate was concentrated. The residue was purified by Flash chromatography (1/4 ethyl acetate/petroleum ether) to provide benzyl 2-(3-(1-(benzyloxycarbonyl)-N-(2-ethoxy-2-oxoethyl)piperidine-2-carboxamido)-1H-pyrrolo[3,2-b]pyridine-1-carbonyl)piperidine-1-carboxylate in 44% yield as a yellow solid.

Triethylamine (14.9 mmol) was added to a solution of benzyl 2-(3-(1-(benzyloxycarbonyl)-1-(2-ethoxy-2-oxoethyl)piperidine-2-carboxamido)-1H-pyrrolo[3,2-b]pyridine-1-carbonyl)piperidine-1-carboxylate (10.8 mmol) in methanol (150 mL) was maintained at rt for 4 h. The reaction mixture was concentrated and the residue was dissolved in dichloromethane (200 mL). The organic layer was washed with water (50 mL), dried (sodium sulfate), and concentrated to provide benzyl 2-((2-ethoxy-2-oxoethyl)(1H-pyrrolo[3,2-b]pyridin-3-yl)carbamoyl)piperidine-1-carboxylate in 83% yield as a yellow oil. Data: LC/MS (ES) m/z 464 [M+H]⁺.

### 3. Synthesis of 2-(1H-pyrrolo[3,2-b]pyridin-3-yl)-hexahydro-6H-pyrido[1,2-a]pyrazine-1,4-dione

A suspension of benzyl 2-((2-ethoxy-2-oxoethyl)(1H-pyrrolo[3,2-b]pyridin-3-yl)carbamoyl)piperidine-1-carboxylate (8.92 mmol) and 10% palladium on carbon (2.5 g) in methanol (150 mL) is maintained under an atmosphere of hydrogen gas for 16 h at 60° C. The catalyst is removed by filtration and the filtrate is concentrated. The residue was purified by Flash chromatography (10/90 methanol/dichloromethane) to provide 2-(1H-pyrrolo[3,2-b]pyridin-3-yl)-hexahydro-6H-pyrido[1,2-a]pyrazine-1,4-dione in 94% yield as a yellow solid. Data: LC/MS (ES) m/z 285 [M+H]⁺.

### 4. Synthesis of 2-(1H-pyrrolo[3,2-b]pyridin-3-yl)-octahydro-1H-pyrido[1,2-a]pyrazine

Lithium aluminum hydride (21.1 mmol) was added to a solution of 2-(1H-pyrrolo[3,2-b]pyridin-3-yl)-hexahydro-6H-pyrido[1,2-a]pyrazine-1,4-dione (3.52 mmol) in tetrahydrofuran (200 mL) at 0° C. The reaction mixture was heated at reflux for 2 h then was allowed to cool to rt and was maintained for 16 h. The reaction mixture was quenched with water (10 mL) and the insoluble solids were removed by filtration. The filtrate was concentrated and the residue was triturated with hexane (2×100 mL) and dried to provide 2-(1H-pyrrolo[3,2-b]pyridin-3-yl)-octahydro-1H-pyrido[1,2-a]pyrazine in 78% yield as an off-white solid. Data: ¹H NMR (CDCl₃) δ 8.46 (s, 1H), 7.94 (s, 1H), 7.61 (d, 1H), 7.11 (s, 1H), 6.89 (s, 1H), 3.89 (m, 4H), 2.92 (t, 3H), 2.67 (t, 1H), 2.50 (t, 1H), 2.36 (t, 1H), 2.18 (t, 2H), 1.35 (t, 3H). LC/MS (ES) m/z 255 [M−H]⁻.

### III. Final Product Preparations

### Procedure 1: Synthesis of 2-[1-(pyridin-3-ylsulfonyl)-1H-pyrrolo[3,2-b]pyridin-3-yl]octahydro-2H-pyrido[1,2-a]pyrazine hydroformate. (compound 16)

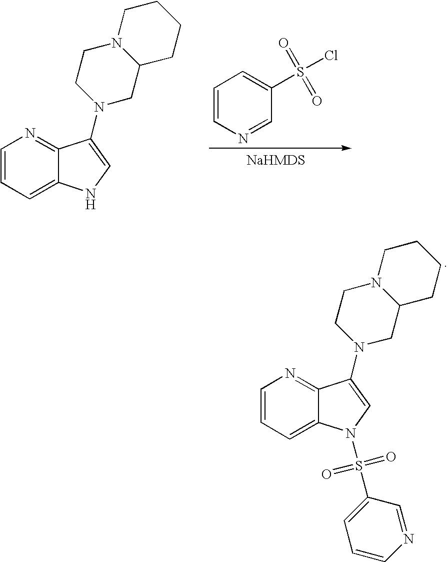

A 1.0 M solution of Sodium bis(trimethylsilyl)amide in tetrahydrofuran (0.28 mmol) was added to a solution of 2-(1H-pyrrolo[3,2-b]pyridin-3-yl)octahydro-2H-pyrido[1,2-a]pyrazine (0.234 mmol) in tetrahydrofuran (4 mL) and N,N-dimethylformamide (1 mL) at 5° C. and the reaction mixture was maintained for 10 min. The reaction mixture was transferred to a second vessel containing a solution of 3-pyridine sulfonyl chloride hydrochloride (0.281 mmol) and N,N-dimethylethylamine (56 mL) in tetrahydrofuran (2 mL). The reaction was maintained for 30 min and was concentrated. The residue was purified by preparative HPLC (Prep Method D on a C₁₈ Atlantis column) and the product containing fractions were pooled and concentrated on a freeze drier to provide 2-[1-(pyridin-3-ylsulfonyl)-1H-pyrrolo[3,2-b]pyridin-3-yl]octahydro-2H-pyrido[1,2-a]pyrazine hydroformate. (16) in 18% yield as a colorless foam. Data: ¹H NMR (CD₃OD) δ 9.1 (d, 1H), 8.7 (d., 1H), 8.5 (d, 1H), 8.4 (d, 1H), 8.3 (d, 1H), 7.5 (m, 1H), 7.4 (m, 2H), 4.1 (m, 2H), 3.2 (m, 2H), 3.0 (m, 2H), 2.9 (t, 1H), 2.6 (m, 2H), 1.7-1.9 (m, 5H), 1.4-1.6 (m, 2H). LC/MS (ES) mil/398.1, t_{R} 3.62 min [M+H]⁺ (Analytical Method C).

Similarly, by using the appropriate starting materials, the following compounds of the invention (compounds 1-15 and 17) were prepared using this procedure; their physical properties are listed in Table 1.

### Procedure 2: Receptor Activity Determination

Assays for determining 5-HT₆ receptor activity, and selectivity of 5-HT₆ receptor activity are known within the art (see. e.g., Example 58 of U.S. Pat. No. 6,903,112).

An assay protocol for determining 5-HT₆ receptor activity generally entailed the incubation of membrane homogenates prepared from HeLa cells expressing the human 5-HT₆ receptor with the radioligand ³H-lysergic acid diethylamide (³H-LSD) at a concentration of 1.29 nM. Concentrations ranging from 10⁻¹ M to 10⁻⁵ M of test compound were incubated with the radioligand and the membrane homogenates. After 60 minutes incubation at 37° C. the reaction was terminated by vacuum filtration. The filters were washed with buffer and were counted for radioactivity using a liquid scintillation counter. The affinity of the test compound was calculated by determining the amount of the compound necessary to inhibit 50% of the binding of the radioligand to the receptor. Ki values were determined based upon the following equation:

Kᵢ=IC₅₀/(1+L/K_{D})

where L is the concentration of the radioligand used and K_{D} is the dissociation constant of the ligand for the receptor (both expressed in nM).

Particular compounds of the invention show 5-HT₆ binding activity with receptor Ki values of typically less than 100 nM, or less than 50 nM. Other compounds have receptor Ki values less than 20 nM. In one embodiment, the Ki value is less than 10 nM, and in another embodiment, less than 5 nM. In yet another embodiment, Ki is less than 3 nM, and in one additional embodiment, Ki is less than 1 nM. In addition, compounds of the invention show 5-HT₆ functional activity with pA2 values of greater than 6 (IC₅₀ less than 1 μM).

In addition, particular compounds of the invention preferably show 5-HT₆ functional activity with 3A4 values where the IC₅₀ is greater than 1 μM, or greater than 3 μM. In another embodiment, it is greater than 10 μM, or greater than 20 μM. Other compounds have an IC₅₀ value for 3A4 is greater than 30 μM. In terms of selectivity, affinity for other serotonin receptors, specifically the 5-HT₁A, 5-HT_{1A}, 5-HT^{1B}, 5-HT_{2A}, 5-HT_{2B}, 5-HT_{2C}, 5-HT_{5A}, and 5-HT₇ receptors, is expressed as the amount (in percent) of binding of the radioligand that is inhibited in the presence of 100 nM test compound. A lower percent inhibition indicates lower affinity for the serotonin receptor. Selected compounds show a percent inhibition of less than 50% for other serotonin receptors. In one embodiment, the compounds show a percent inhibition of less than 25% for other serotonin receptors.

In another embodiment, the particular compounds show both 5-HT₆ binding activity with a low receptor Ki values and a high IC₅₀ value for 3A4 in a 5-HT₆ functional activity. Compounds with a significantly low receptor Ki value (e.g., less than 10 nM or less than 3 nM) can have lower 3A4 values (e.g., a compounds with a Ki value of less than 3 nM but a 3A4 value of only less than 3 μM is a preferred compound for one embodiment of the invention.)

The preceding procedures and examples can be repeated with similar success by substituting the generically or specifically described reactants and/or operating conditions of this invention for those used in the preceding procedures and examples.

While the invention has been illustrated with respect to the production and of particular compounds, it is apparent that variations and modifications of the invention can be made without departing from the spirit or scope of the invention. Upon further study of the specification, further aspects, objects and advantages of this invention will become apparent to those skilled in the art.

We claim:

1. A compound having the formula: 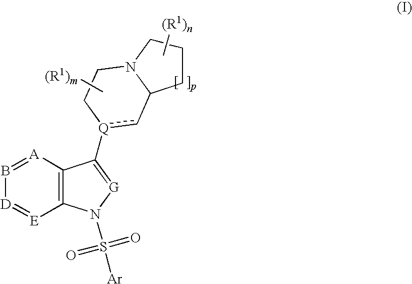
wherein
A, B, D, E and G are each N, CH or CR², wherein at least one of A, B, D, E and G is N;
Q is independently N, C or CH; [symbol]
represents a single bond or a double bond;
m is 0, 1, or 2;
n is 0, 1, or 2;
p is 1, 2 or 3;
R¹ is, in each instance independently, H, OH, oxo, phenyl, or heteroaryl, alkyl having 1 to 8 carbon atoms, or alkoxy having 1 to 8 carbon atoms, each of which is unsubstituted or substituted one or more times with halogen, OH, C₁₋₄-alkyl, C₁₋₄-alkoxy, oxo, phenyl, heteroaryl, or any combination thereof;
R² is, in each instance independently, halogen, nitro, cyano, or NR³R³,
alkyl having 1 to 8 carbon atoms, which is unsubstituted or substituted one or more times with halogen, C₁₋₄-alkyl, C₁₋₄-alkoxy, oxo, or any combination thereof,
alkoxy having 1 to 8-carbon atoms, which is unsubstituted or substituted one or more times with halogen, C₁₋₆ alkyl, phenyl, or heteroaryl,
an aryl group having 6 to 10 ring atoms, which is unsubstituted or substituted one or more times by halogen, hydroxy, C₁₋₄-alkyl, C₁₋₄-alkoxy, cyano, halogenated C₁₋₄-alkyl, nitro, or any combination thereof,
a heterocyclic group, which is saturated, partially saturated or unsaturated, having 5 to 10 ring atoms in which at least 1 ring atom is an N, O or S atom, which is unsubstituted or substituted one or more times by halogen, hydroxy, C₆₋₁₀-aryl, C₁₋₄-alkyl, C₁₋₄-alkoxy, cyano, halogenated C₁₋₄-alkyl, nitro, or any combination thereof,
—C(═O)alkyl, —C(═O)-heteroaryl, —C(═O)—NR⁷R⁷, —C(═O)-alkoxy, or —C(═O)-phenyl;
R³ is, in each instance, independently hydrogen,
alkyl having 1 to 8 carbon atoms, cycloalkyl having 3 to 12 carbon atoms, or cycloalkylalkyl having 4 to 12 carbon atoms, each of which is branched or unbranched and which is unsubstituted or substituted one or more times with halogen, C₁₋₄-alkyl, C₁₋₄-alkoxy, oxo, or any combination thereof,
an aryl group having 6 to 10 ring atoms, which is unsubstituted or substituted one or more times by halogen, hydroxy, C₅₋₇-aryl, C₁₋₄-alkyl, C₁₋₄-alkoxy, cyano, halogenated C₁₋₄-alkyl, nitro, or any combination thereof,
a heterocyclic group, which is saturated, partially saturated or unsaturated, having 5 to 10 ring atoms in which at least 1 ring atom is an N, O or S atom, which is unsubstituted or substituted one or more times by halogen, hydroxy, C₅₋₇-aryl, C₁₋₄-alkyl, C₁₋₄-alkoxy, cyano, halogenated C₁₋₄-alkyl, nitro, or any combination thereof,
aralkyl, heteroaralkyl, heterocyclylalkyl, —C(O)R⁶, —C(O)OR⁷, —C(O)NR⁷R⁷, —S(O)R⁶, or —S(O)₂R⁶;
Ar is selected from Formulas (a), (b), and (c): 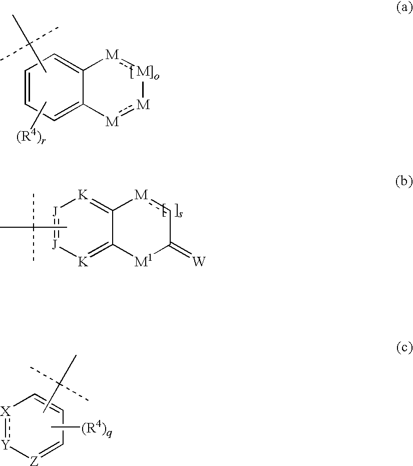
J is, in each instance independently C (attachment to sulfonyl), CH, or CR⁴;
K is, in each instance independently C (attachment to sulfonyl), CH, CR⁴ or N;
M is, in each instance independently CH, CH₂, CR⁴, CHR⁴, N, NR⁵, O or S, wherein at least one M is not CH, CH₂, CR⁴, or CHR⁴;
M¹ is CH₂, CHR⁴, NR⁵, O or S;
X, Y, and Z are each independently C (attachment to sulfonyl), CH, CR⁴, or N;
W is O, S or is absent;
o is 0 or 1;
q is 0, 1 or 2;
r is 0, 1, 2 or 3;
s is 0 or 1; [symbol]
represents a single bond or a double bond;
R⁴ is, in each instance, independently, halogen, C₁₋₄-alkyl, C₁₋₄-alkoxy, —C(═O)alkyl, —C(═O)-pyridyl, cyano, amino, N—C₁₋₄-alkyl-N—C₁₋₄-acylamino, mono- or di-C₁₋₄-alkylamino, morpholinyl, pyrrolidinyl, or pyrrolidone-1-yl; wherein
the morpholinyl, pyrrolidinyl, or pyrrolidone-1-yl group may be substituted with hydroxy, C₁₋₈-alkyl, C₁₋₄-alkoxy, or a 3 to 7-membered heterocycloalkoxy containing at least one O, S or N atom, and wherein
each alkyl and alkoxy is independently unsubstituted or substituted one or more times by halogen or acyl;
R⁵ is, in each instance, independently hydrogen,
alkyl having 1 to 8 carbon atoms, cycloalkyl having 3 to 12 carbon atoms, alkenyl or alkynyl having 3 to 8 and at least one double or triple bond located at least one carbon atom from the point of attachment, cycloalkylalkyl having 4 to 12 carbon atoms, each of which is branched or unbranched and which is unsubstituted or substituted one or more times with halogen, C₁₋₄-alkyl, C₁₋₄-alkoxy, oxo, or any combination thereof,
an aryl group having 6 to 10 ring atoms, which is unsubstituted or substituted one or more times by halogen, hydroxy, C₅₋₇-aryl, C₁₋₄-alkyl, C₁₋₄-alkoxy, cyano, halogenated C₁₋₄-alkyl, nitro, or any combination thereof,
a heterocyclic group, which is saturated, partially saturated or unsaturated, having 5 to 10 ring atoms in which at least 1 ring atom is an N, O or S atom, which is unsubstituted or substituted one or more times by halogen, hydroxy, C₅₋₇-aryl, C₁₋₄-alkyl, C₁₋₄-alkoxy, cyano, halogenated C₁₋₄-alkyl, nitro, or any combination thereof,
C₇₋₁₄-aralkyl, C₅₋₁₃-heteroaralkyl, C₅₋₁₃-heterocyclylalkyl, —C(O)R⁶, —C(O)OR⁷, —C(O)NR⁷R⁷, —S(O)R⁶, or —S(O)₂R⁶;
R⁶ is, in each instance, independently C₁₋₄-alkyl, C₃₋₈-cycloalkyl, C₄₋₁₀-cycloalkylalkyl, C₆₋₁₀-aryl, C₇₋₁₄-aralkyl, C₄₋₇-heteroaryl, C₅₋₁₃-heteroaralkyl, C₄₋₉-heterocyclyl, or C₅₋₁₃-heterocyclylalkyl;
R⁷ is, in each instance, independently hydrogen, C₁₋₄-alkyl, C₃₋₈-cycloalkyl, C₄₋₁₀-cycloalkylalkyl, C₆₋₁₀-aryl, C₇₋₁₄-aralkyl, C₄₋₇-heteroaryl, C₅₋₁₃-heteroaralkyl, C₄₋₉-heterocyclyl, or C₅₋₁₃-heterocyclylalkyl;
or a pharmaceutically acceptable salt or solvate thereof, or a solvate of a pharmaceutically acceptable salt thereof,
provided that when Ar is (c) and X, Y and Z are each CH or CR⁴, then q is 1 or 2 and at least one R⁴ is C(═O)alkyl, —C(═O)pyridyl, cyano, amino, N—C₁₋₄-alkyl-N-acylamino, mono- or di-C₁₋₄-alkylamino, morpholinyl, pyrrolidinyl, or pyrrolidone-1-yl, wherein the alkyl may be substituted or unsubstituted.

2. The compound of claim 1, wherein the compound contains a chiral center and is a racemic mixture.

3. The compound of claim 1, wherein compound contains a chiral center and is substantially the [S] isomer at the chiral center.

4. The compound of claim 1, wherein the compound contains a chiral center and is the substantially the [R] isomer at the chiral center.

5. The compound of claim 1, comprising at least two R⁴ moieties on the Ar of Formula (a), (b), or (c), wherein the two R⁴s are the same.

6. The compound of claim 1, wherein Ar is (b) and both variable J are CH.

7. The compound of claim 1, wherein G is N.

8. The compound of claim 1, wherein A is N and B, D, E, and G are CH or CR².

9. The compound of claim 1, wherein q is 1, 2, or 3 or wherein p is 1 or 2.

10. The compound of claim 1, wherein R¹ is H or wherein each R² is H.

11. The compound of claim 1 comprising at least one R⁴, wherein at least one R⁴ is a heterocyclic group.

12. The compound of claim 1, wherein Ar is (a), one M is O, and o is 1.

13. The compound of claim 1, wherein Ar is (b), one M is NH and the other M is O, W is ═O, and p is 1.

14. The compound of claim 1, wherein Ar is (c) and A is N.

15. The compound of claim 1, wherein Ar is (c), A is N, and each R⁴ on the ring in formula (c) is independently H, a halogen, a C₁-C₄ alkyl, a C₁-C₄ alkoxy, cyano, or a substituted or unsubstituted heterocyclic group.

16. The compound of claim 1, wherein Ar is (c), A is N, q is 1 or 2, and at least one R⁴ on the ring in formula (c) is a cyano group.

17. The compound of claim 1, wherein Ar is (a) or (b) and at least one [symbol] in the ring of (a) or (b) is a single bond.

18. The compound of claim 1, wherein Ar is (a) or (b), and (a) or (b) contains at least three ring heteroatoms.

19. The compound of claim 1, wherein Ar is (b) and W is present.

20. The compound of claim 1, wherein M is, in each instance independently CH, CH₂, N, NR⁵, O or S, wherein at least one M is not CH or CH₂.

21. The compound of claim 1, wherein Ar is (a), (b) or (c) wherein (a), (b), and (c) are represented by the formulas (a′), (b′), and (c′):

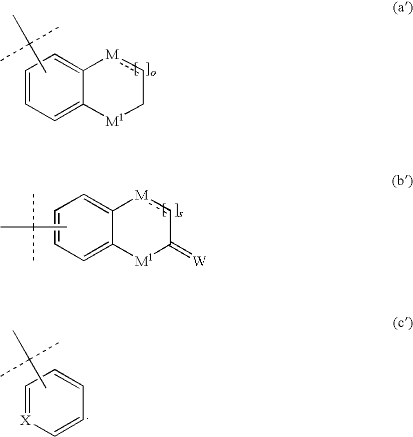

22. The compound of claim 1, wherein Ar is (c), X, Y, and Z are each CH, q is 1, and R⁴ in formula (c) is a pyrrolidine or substituted pyrrolidine.

23. The compound of claim 1, wherein Ar contains at least one R⁴ wherein R⁴ is C(═O)alkyl, —C(═O)pyridyl, cyano, amino, N—C₁₋₄-alkyl-N—C₁₋₄-acylamino, mono- or di-C₁₋₄-alkylamino, morpholinyl, pyrrolidinyl, or pyrrolidonyl, wherein the alkyl, morpholinyl, pyrrolidinyl, or pyrrolidonyl may be substituted or unsubstituted.

24. The compound of claim 1, wherein the compound is a hydroformate salt, a phosphate salt, a dihydroiodide, a dihydrochloride monohydrate, or a hydroacetate salt.

25. The compound of claim 1, wherein the compound of formula (I) comprises a 1-H-pyrrolo[3,2-b]pyridine moiety.

26. The compound of claim 1, wherein the compound of formula (I) is defined by the compound of formula (II): 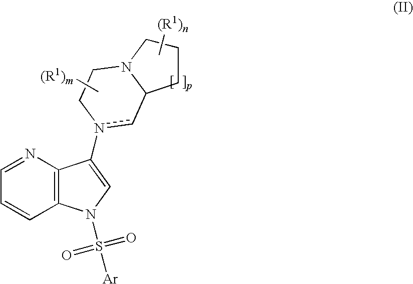
wherein R¹, Ar, n, m, and p are defined as above.

27. The compound of claim 1, where the compound is selected from the group consisting of:
3-(hexahydropyrrolo[1,2-a]pyrazin-2(1H)-yl)-1-(phenylsulfonyl)-1H-pyrrolo[3,2-b]pyridine,
1-[(3-chlorophenyl)sulfonyl]-3-(hexahydropyrrolo[1,2-a]pyrazin-2(1H)-yl)-1H-pyrrolo[3,2-b]pyridine,
1-[(2-chlorophenyl)sulfonyl]-3-(hexahydropyrrolo[1,2-a]pyrazin-2(1H)-yl)-1H-pyrrolo[3,2-b]pyridine,
1-[(3-fluorophenyl)sulfonyl]-3-(hexahydropyrrolo[1,2-a]pyrazin-2(1H)-yl)-1H-pyrrolo[3,2-b]pyridine,
1-[(2-fluorophenyl)sulfonyl]-3-(hexahydropyrrolo[1,2-a]pyrazin-2(1H)-yl)-1H-pyrrolo[3,2-b]pyridine,
3-(hexahydropyrrolo[1,2-a]pyrazin-2(1H)-yl)-1-[(3-methoxyphenyl)sulfonyl]-1H-pyrrolo[3,2-b]pyridine,
3-(hexahydropyrrolo[1,2-a]pyrazin-2(1H)-yl)-1-[(2-methoxyphenyl)sulfonyl]-1H-pyrrolo[3,2-b]pyridine,
3-{[3-(hexahydropyrrolo[1,2-a]pyrazin-2(1H)-yl)-1H-pyrrolo[3,2-b]pyridin-1-yl]sulfonyl}benzonitrile, and
2-{[3-(hexahydropyrrolo[1,2-a]pyrazin-2(1H)-yl)-1H-pyrrolo[3,2-b]pyridin-1-yl]sulfonyl}-4-methylbenzonitrile,
or a pharmaceutically acceptable salt, a pharmaceutically acceptable solvate, or a solvate of a pharmaceutically acceptable salt thereof.

28. The compound of claim 1, where the compound is selected from the group consisting of:
1-(2,3-dihydro-1-benzofuran-6-ylsulfonyl)-3-(hexahydropyrrolo[1,2-a]pyrazin-2(1H)-yl)-1H-pyrrolo[3,2-b]pyridine,
1-(2,3-dihydro-1-benzofuran-4-ylsulfonyl)-3-(hexahydropyrrolo[1,2-a]pyrazin-2(1H)-yl)-1H-pyrrolo[3,2-b]pyridine,
2-[1-(phenylsulfonyl)-1H-pyrrolo[3,2-b]pyridin-3-yl]octahydro-2H-pyrido[1,2-a]pyrazine,
2-{1-[(2-chlorophenyl)sulfonyl]-1H-pyrrolo[3,2-b]pyridin-3-yl}octahydro-2H-pyrido[1,2-a]pyrazine,
2-{1-[(3-chlorophenyl)sulfonyl]-1H-pyrrolo[3,2-b]pyridin-3-yl}octahydro-2H-pyrido[1,2-a]pyrazine,
2-{1-[(4-chlorophenyl)sulfonyl]-1H-pyrrolo[3,2-b]pyridin-3-yl}octahydro-2H-pyrido[1,2-a]pyrazine,
2-[1-(pyridin-3-ylsulfonyl)-1H-pyrrolo[3,2-b]pyridin-3-yl]octahydro-2H-pyrido[1,2-a]pyrazine, and
2-{1-[(3-fluorophenyl)sulfonyl]-1H-pyrrolo[3,2-b]pyridin-3-yl}octahydro-2H-pyrido[1,2-a]pyrazine,
or a pharmaceutically acceptable salt, a pharmaceutically acceptable solvate, or a solvate of a pharmaceutically acceptable salt thereof.

29. The compound according to claim 1, selected from the group consisting of:
1-[(2-chlorophenyl)sulfonyl]-3-(hexahydropyrrolo[1,2-a]pyrazin-2(1H)-yl)-1H-pyrrolo[3,2-b]pyridine,
3-(hexahydropyrrolo[1,2-a]pyrazin-2(1H)-yl)-1-[(3-methoxyphenyl)sulfonyl]-1H-pyrrolo[3,2-b]pyridine, and
1-[(3-chlorophenyl)sulfonyl]-3-(hexahydropyrrolo[1,2-a]pyrazin-2(1H)-yl)-1H-pyrrolo[3,2-b]pyridine,
or a pharmaceutically acceptable salt, a pharmaceutically acceptable solvate, or a solvate of a pharmaceutically acceptable salt thereof.

30. The compound of claim 28, wherein the pharmaceutically acceptable salt is a hydroformate salt.

31. A pharmaceutical composition comprising a therapeutically effective amount of the compound of claim 1 and a pharmaceutically acceptable carrier.

32. A method of modulating 5-HT₆ receptor activity comprising administering a pharmacologically effective amount of a compound according to claim 1 to a patient in need thereof.

33. The method of claim 32, further comprising treating a central nervous system (CNS) disorder, a memory/cognitive impairment, withdrawal from drug abuse, psychoses, a gastrointestinal (GI) disorder, or a polyglutamine-repeat disease by administering a pharmacologically effective amount of a compound according to claim 1 to a patient in need thereof.

34. The method of claim 33, wherein:
the CNS disorder is Alzheimer's disease, Parkinson's disease, Huntington's disease, anxiety, depression, manic depression, epilepsy, obsessive compulsive disorders, migraine, sleep disorders, feeding disorders such as anorexia and bulimia, panic attacks, attention deficit hyperactivity disorder (ADHD), attention deficit disorder (ADD), withdrawal from drug abuse, psychoses, or disorders associated with spinal trauma and/or head injury;
the memory/cognitive impairment is associated with Alzheimer's disease, schizophrenia, Parkinson's disease, Huntington's disease Pick's disease, Creutzfeld Jakob disease, HIV, cardiovascular disease, head trauma or age-related cognitive decline; or
the GI disorder is functional bowel disorder, constipation, gastroesophageal reflux disease (GERD), nocturnal-GERD, irritable bowel syndrome (IBS), constipation-predominant IBS (IBS-c) or alternating constipation/diarrhea IBS.

35. The method of claim 33, wherein the disorder is Alzheimer's disease.

36. The method of claim 33, wherein the disorder is attention deficit disorder (ADD).

37. The method of claim 33, wherein the disorder schizophrenia.

38. The method of claim 33, further comprising treating obesity by administering a pharmacologically effective amount of a compound according to claim 1 to a patient in need thereof.

39. The method of claim 32, wherein the compound of claim 1 is administered in a pharmaceutically acceptable carrier.
